(19) United States
(12) Patent Application Publication
(10) Pub. No.: US 2021/0107989 A1
(43) Pub. Date: Apr. 15, 2021

(54) METHODS FOR TREATING CD73HI TUMORS

(71) Applicant: CORVUS PHARMACEUTICALS, INC., Burlingame, CA (US)

(72) Inventors: Emily Piccione GRIFFIN, Belmont, CA (US); Ian MCCAFFERY, Oakland, CA (US)

(21) Appl. No.: 16/500,654

(22) PCT Filed: Apr. 4, 2018
(86) PCT No.: PCT/US2018/026142

§ 371 Date: Oct. 3, 2019

Related U.S. Application Data
(60) Provisional application No. 62/481,660, filed on Apr. 4, 2017.

(51) Int. Cl. C07K 16/28; A61P 35/00; A61K 31/52

(52) U.S. Cl. CPC C07K 16/2896; A61K 2039/505; A61K 31/52; A61P 35/00

(57) ABSTRACT

Provided herein are, inter alia, 1E9 antibodies capable of binding CD73. The humanized antibodies are useful for the treatment of cancer in a subject expressing elevated levels of CD73.

### CROSS-REFERENCES TO RELATED APPLICATIONS

This application claims the benefit of U.S. Provisional Application No. 62/481,660, filed Apr. 4, 2017, which is incorporated herein by reference in entirety and for all purposes

### REFERENCE TO A “SEQUENCE LISTING,” A TABLE, OR A COMPUTER PROGRAM LISTING APPENDIX SUBMITTED AS AN ASCII FILE

The Sequence Listing written in file 048517-529001WO Sequence Listing ST25.txt, created Mar. 29, 2018, 50,284 bytes, machine format IBM-PC, MS Windows operating system, is hereby incorporated by reference.

### BACKGROUND OF THE INVENTION

The glycosyl-phosphatidylinositol-anchored CD73 antigen is considered the rate-limiting enzyme in the generation of extracellular adenosine (Stagg J, Smyth M J. Extracellular adenosine triphosphate and adenosine in cancer. Oncogene. 2010; 29:5346-58. doi: 10.1038/onc.2010.292). CD73 can be found constitutively expressed at high levels on various types of cancer cells. CD73-generated adenosine is assumed to suppress adaptive anti-tumor immune responses thereby promoting tumor growth and metastasis. There is a need in the art for antibody-based CD73 cancer therapy which inhibits the catalytic activity of CD73 and prevents the ability of circulating tumor cells to extravasate and colonize thereby inhibiting metastasis. The present invention addresses these and other needs in the art.

### BRIEF SUMMARY OF THE INVENTION

In one aspect a method of treating cancer in a subject in need thereof is provided. The method includes administering a therapeutically effective amount of a humanized 1E9 antibody wherein the subject expresses an elevated level of CD73 relative to a standard control and wherein the 1E9 antibody includes a humanized light chain variable region including a mouse CDR L1, mouse CDR L2, or mouse CDR L3 and a humanized heavy chain variable region including a mouse CDR H1, mouse CDR H2, or mouse CDR H3.

In one aspect is provided a method of treating cancer in a subject in need thereof. The method includes administering to the subject a therapeutically effective amount of a 1E9 antibody, wherein the subject expresses an elevated level of CD73 relative to a standard control, and wherein the 1E9 antibody includes (i) a mouse CDR L1 as set forth in SEQ ID NO:1, a mouse CDR L2 as set forth in SEQ ID NO:2, a mouse CDR L3 as set forth in SEQ ID NO:3; and (ii) a mouse CDR H1 as set forth in SEQ ID NO:4, a mouse CDR H2 as set forth in SEQ ID NO:5, and a mouse CDR H3 as set forth in SEQ ID NO:6.

In one aspect is provided a method of treating cancer in a subject in need thereof. The method includes administering to the subject a therapeutically effective amount of a humanized IgG1 antibody, wherein the subject expresses an elevated level of CD73 relative to a standard control and wherein the humanized IgG1 antibody includes a humanized light chain variable region and a humanized heavy chain variable region, wherein the humanized light chain variable region includes a mouse CDR L1 as set forth in SEQ ID NO:1, a mouse CDR L2 as set forth in SEQ ID NO:2, a mouse CDR L3 as set forth in SEQ ID NO:3; and wherein the humanized heavy chain variable region includes a mouse CDR H1 as set forth in SEQ ID NO:4, a mouse CDR H2 as set forth in SEQ ID NO:5, and a mouse CDR H3 as set forth in SEQ ID NO:6.

In one aspect, provided herein is a humanized IgG4 antibody including a humanized light chain variable region and a humanized heavy chain variable region, wherein the humanized light chain variable region includes a mouse CDR L1 as set forth in SEQ ID NO:1, a mouse CDR L2 as set forth in SEQ ID NO:2, a mouse CDR L3 as set forth in SEQ ID NO:3 and wherein the humanized heavy chain variable region includes a mouse CDR H1 as set forth in SEQ ID NO:4, a mouse CDR H2 as set forth in SEQ ID NO:5, and a mouse CDR H3 as set forth in SEQ ID NO:6.

In another aspect is provided a method of treating cancer in a subject in need thereof. The method includes administering to the subject a therapeutically effective amount of an anti-CD73 antibody, wherein the subject expresses an elevated level of CD73 relative to a standard control and wherein the anti-CD73 antibody binds the same epitope as a 1E9 antibody.

### BRIEF DESCRIPTION OF THE DRAWINGS

FIGS. 1A-1B: CPX-006 and CPX-016 bind to CD73 and reduce adenosine production. FIG. 1A: MDA-MB-231 cells were incubated with CPX-006 or CPX-016 over a range of concentrations and antibody binding was detected with a PE-conjugated secondary antibody and flow cytometry analysis. Mean fluorescence intensity (MFI) of PE signal is reported. FIG. 1B: MDA-MB-231 parental cells or CD73 CRISPR knockout cells were incubated with the indicated antibodies or APCP, a small molecule inhibitor of CD73 enzymatic activity, prior to addition of 250 μM AMP. Phosphate levels were measured in the cell culture supernatant using the Sensolyte Malachite Green phosphate assay kit.

FIGS. 2A-2C: CPX-006 and CPX-016 Bind to Distinct Epitopes on CD73. FIG. 2A: Schematic of experimental design. FIG. 2B: Pre-incubated cells were subsequently incubated with a titration of CPX-006 prior to staining with PE-conjugated anti-human secondary antibody and antibody binding was assessed by flow cytometry and mean fluorescence intensity (MFI) for PE was determined. FIG. 2C: Pre-incubated cells were subsequently incubated with a titration of CPX-016 prior to staining with PE-conjugated anti-human secondary antibody and antibody binding was assessed by flow cytometry and mean fluorescence intensity (MFI) for PE was determined.

FIGS. 3A-3E: CPX-006 Reverses Suppressive Effects of AMP on T cell activity. FIG. 3A: Schematic of experimental design. FIG. 3B: T cell proliferation for a representative donor. FIG. 3C: T cell proliferation results for 13 donors treated with 500 nM CPX-006 or CPX-016 or 890 nM isotype control. FIG. 3D: IFN-gamma production (reported as raw AlphaLISA values) for a representative donor. FIG. 3E: IFN-gamma production for 6 donors treated with 500 nM CPX-006 or CPX-016 or 890 nM isotype control.

FIGS. 4A-4D: CPX-006 reverses suppressive effects of AMP across a broad range of CD73 expression. FIG. 4A: CD73 expression levels in each donor were determined by flow cytometry and were plotted as a function of the effect of CPX-006 on restoring T cell proliferation in the presence of AMP as reported in FIG. 3C. FIG. 4B: CD73 expression levels in each donor were determined by flow cytometry and were plotted as a function of the effect of CPX-016 on restoring T cell proliferation in the presence of AMP as reported in FIG. 3C. FIG. 4C: MDA-MB-231 cells were engineered to reduce or eliminate CD73 expression via shRNA or CRISPR, respectively. CD73 protein levels were measured by flow cytometry analysis. FIG. 4D: CD73 catalytic activity was measured for each cell line in the presence of 1 μM CPX-006 or CPX-016 or 1 mM APCP as described FIG. 1B.

FIGS. 5A-5D: CD73 is highly expressed in multiple tumor types. CD73 expression was analyzed by immunohistochemical staining of tissues from renal cell cancer (n=62), NSCLC (n=68), melanoma (n=68), and breast cancer (n=94). Six representative tissues are shown from rencal cell cancer (FIG. 5A), melanoma (FIG. 5B), breast cancer (FIG. 5C), and lung cancer (FIG. 5D).

FIGS. 6A-6D: CPX-006 Augments CPI-444 to Reverse AMP-mediated Suppression of T Cell Proliferation. FIG. 6A: Percentage of CD3+ T cell proliferation in a representative donor in response to treatment with isotype and vehicle, CPX-006, CPI-444, or CPX-006 and CPI-444 (10 μM). Dotted lines indicate percentage of CD3+ T cell proliferation in the presence of no AMP or 3 mM AMP. FIG. 6B: T cell proliferation for 10 donors treated with 500 nM CPX-006, 10 μM CPI-444, 500 nM CPX-006+10 μM CPI-444, or vehicle (890 nM isotype control+0.1% DMSO). FIG. 6C: IFNg secretion as evidence by fluorescence intensity in response to treatment with vehicle (isotype and 0.1% DMSO), CPX-006, CPI-444, or CPX-006 and CPI-444 (10 μM). Dotted lines indicate IFNg secretion as evidence by fluorescence intensity in the presence of no AMP or 3 mM AMP. FIG. 6D: IFN-gamma production (reported as raw AlphaLISA values) for 6 donors treated as described in FIG. 6B.

FIGS. 7A-7B: Complete Occupancy Achieved in Repeat-Dose Tox Study with No Observed Toxicities. FIG. 7A: Plot demonstrating PD (free CD73 on CD8+ T cells) as a function of PK (ng/mL) for two studies. Study 1 (Groups: 3, 10, 30 mg/kg) and Study 2 (Groups: 10, 40, 120 mg/kg) demonstrated complete occupancy (dotted line) with repeat dosing. FIG. 7B: CD73 occupancy was determined by measuring ex vivo staining of CD8+ T cells with Alexa-Fluor 647 labeled CPX-006. Ratio of AF647 staining on CD73+/CD73− cells is reported.

FIGS. 8A-8D: All Tumor Samples From Major IO Indications Express CD73. Expression is predominantly in the stroma. Scatter plots showing relationship between tumor cell membranous H-scores (tumor score) and the percentage of CD73 positive stroma per tumor surface (stromal score) for melanoma tissue samples (FIG. 8A), lung cancer tissue samples (FIG. 8B; squares represent values for squamous cell carcinoma tissue samples and circles represent values for adenocarcinoma), renal cancer tissue samples (FIG. 8C), and breast cancer tissue samples (FIG. 8D).

FIG. 9. CD73 (NT5E gene) expression levels in each PBMC donor were determined by Nanostring and were plotted as a function of the effect of CPX-006 or CPX-016 (500 nM) on restoring T cell proliferation in the presence of AMP. CD73 (NT5E) expression levels were also measured for NSCLC squamous and adenocarcinoma tumors and MDA-MB-231 TNBC cell line. All expression levels were determined by Nanostring analysis.

### DETAILED DESCRIPTION OF THE INVENTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The terms “CDR L1”, “CDR L2” and “CDR L3” as provided herein refer to the complementarity determining regions (CDR) 1, 2, and 3 of the variable light (L) chain of an antibody. Likewise, the terms “CDR H1”, “CDR H2” and “CDR H3” as provided herein refer to the complementarity determining regions (CDR) 1, 2, and 3 of the variable heavy (H) chain of an antibody.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3-d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A “ligand” refers to an agent, e.g., a polypeptide or other molecule, capable of binding to a receptor.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a biotin domain as described herein and a biotin-binding domain. In embodiments contacting includes, for example, allowing a humanized antibody as described herein to interact with CD73 antigen.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length. The present invention includes polypeptides that are substantially identical to any of SEQ ID NOs:30-51.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

An amino acid residue in an antibody “corresponds” to a given residue when it occupies the same essential structural position within the antibody as the given residue. For example, a selected residue in a comparison antibody corresponds to position 48 (according to the Kabat numbering system as described herein) in an antibody provided herein when the selected residue occupies the same essential spatial or structural relationship to Kabat position 48 as assessed using applicable methods in the art. For example, a comparison antibody may be aligned for maximum sequence homology with the antibody provided herein and the position in the aligned comparison antibody that aligns with Kabat position 48 may be determined to correspond to it. Alternatively, instead of (or in addition to) a primary sequence alignment as described above, a three dimensional structural alignment can also be used, e.g., where the structure of the comparison antibody is aligned for maximum correspondence with an antibody provided herein and the overall structures compared. In this case, an amino acid that occupies the same essential position as Kabat position 48 in the structural model may be said to correspond.

The term “isolated,” when applied to a protein, denotes that the protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a protein gives rise to essentially one band in an electrophoretic gel. Particularly, it means that the protein is at least 85% pure, more preferably at least 95% pure, and most preferably at least 99% pure.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Typically a specific or selective reaction will be at least twice background signal or noise and more typically more than 10 to 100 times background.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

As defined herein, the term “inhibition”, “inhibit”, “inhibiting” and the like in reference to a protein-inhibitor (e.g., an anti-CD73 antibody, an 1E9 antibody, IgG1 antibody or humanized 1E9 antibody) interaction means negatively affecting (e.g., decreasing) the activity or function of the protein (e.g., decreasing the catalytic activity of CD73) relative to the activity or function of the protein in the absence of the inhibitor (e.g., an anti-CD73 antibody, an 1E9 antibody, IgG1 antibody or humanized 1E9 antibody). In some embodiments inhibition refers to reduction of a disease or symptoms of disease. Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits CD73 activity, e.g., by binding, partially or totally blocking, decreasing, preventing, delaying, inactivating, desensitizing, or down-regulating enzymatic activity (e.g., CD73 catalytic activity).

Agents of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

The compositions can be administered for therapeutic or prophylactic treatments. In therapeutic applications, compositions are administered to a patient suffering from a disease (e.g., cancer) in a “therapeutically effective dose.” Amounts effective for this use will depend upon the severity of the disease and the general state of the patient's health. Single or multiple administrations of the compositions may be administered depending on the dosage and frequency as required and tolerated by the patient. A “patient” or “subject” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The compositions provided herein, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administrations contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). The disease may be an autoimmune, inflammatory, cancer, infectious, metabolic, developmental, cardiovascular, liver, intestinal, endocrine, neurological, or other disease.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) is caused by (in whole or in part), or a symptom of the disease is caused by (in whole or in part) the substance or substance activity or function.

A “control” sample or value refers to a sample that serves as a reference, usually a known reference, for comparison to a test sample. For example, a test sample can be taken from a patient suspected of having a given disease (e.g., cancer) and compared to samples from a known cancer patient, or a known normal (e.g., non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched non-cyclic chain, or combinations thereof, including at least one carbon atom and at least one heteroatom (e.g. O, N, P, Si, and S), and wherein the nitrogen and sulfur atoms may optionally be oxidized, and the nitrogen heteroatom may optionally be quaternized. The heteroatom(s) (e.g. O, N, P, S, and Si) may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl”, “heteroalkyl”, “cycloalkyl”, “heterocycloalkyl”, “aryl”, and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Provided herein are agents (e.g. compounds, drugs, therapeutic agents) that may be in a prodrug form. Prodrugs of the compounds described herein are those compounds that readily undergo chemical changes under select physiological conditions to provide the final agents (e.g. compounds, drugs, therapeutic agents). Additionally, prodrugs can be converted to agents (e.g. compounds, drugs, therapeutic agents) by chemical or biochemical methods in an ex vivo environment. Prodrugs described herein include compounds that readily undergo chemical changes under select physiological conditions to provide agents (e.g. compounds, drugs, therapeutic agents) to a biological system (e.g. in a subject).

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

In embodiments, a compound as described herein may include multiple instances of R² and/or other variables. In such embodiments, each variable may optional be different and be appropriately labeled to distinguish each group for greater clarity. For example, where each R² is different, they may be referred to, for example, as R^{2.1}, R^{2.2}, R^{2.3}, and/or R^{2.4} respectively, wherein the definition of R² is assumed by R^{2.1}, R^{2.2}, R^{2.3}, and/or R^{2.4}. The variables used within a definition of R² and/or other variables that appear at multiple instances and are different may similarly be appropriately labeled to distinguish each group for greater clarity. In some embodiments, the compound is a compound described herein (e.g., in an aspect, embodiment, example, claim, table, scheme, drawing, or figure).

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. For example, where a moiety herein is R¹²-substituted or unsubstituted alkyl, a plurality of R¹² substituents may be attached to the alkyl moiety wherein each R¹² substituent is optionally different. Where an R-substituted moiety is substituted with a plurality R substituents, each of the R-substituents may be differentiated herein using a prime symbol (′) such as R″, etc. For example, where a moiety is R¹²-substituted or unsubstituted alkyl, and the moiety is substituted with a plurality of R¹² substituents, the plurality of R¹² substituents may be differentiated as R¹²′, R¹²″, R¹²′″, etc. In embodiments, the plurality of R substituents is 3. In embodiments, the plurality of R substituents is 2.

In embodiments, a compound as described herein may include multiple instances of R¹, R², R³, R⁴, R⁵, R⁶, R⁷, R⁹, R¹⁰, R¹¹, R¹², R¹³, R¹⁴ and/or other variables. In such embodiments, each variable may optional be different and be appropriately labeled to distinguish each group for greater clarity. For example, where each R¹, R², R³, R⁴, R⁵, R⁶, R⁷, R⁹, R¹⁰, R¹¹, R¹², R¹³, and/or R¹⁴, is different, they may be referred to, for example, as R^{1.1}, R^{1.2}, R^{1.3}, R^{1.4}, R^{2.1}, R^{2.2}, R^{2.3}, R^{2.4}, R^{3.1}, R^{3.2}, R^{3.3}, R^{3.4}, R^{4.1}, R^{4.2}, R^{4.3}, R^{4.4}, R^{5.1}, R^{5.2}, R^{5.3}, R^{5.4}, R^{6.1}, R^{6.2}, R^{6.3}, R^{6.4}, R^{7.1}, R^{7.2}, R^{7.3}, R^{7.4}, R^{9.1}, R^{9.2}, R^{9.3}, R^{9.4}, R^{10.1}, R^{10.2}, R^{10.3}, R^{10.4}, R^{11.1}, R^{11.2}, R^{11.3}, R^{11.4}, R^{12.1}, R^{12.2}, R^{12.3}, R^{12.4}, R^{13.1}, R^{13.2}, R^{13.3}, R^{13.4}, R^{14.1}, R^{14.2}, R^{14.3}, and/or R^{14.4}, respectively, wherein the definition of R¹ is assumed by R^{1.1}, R^{1.2}, R^{1.3}, and/or R^{1.4}, the definition of R² is assumed by R^{2.1}, R^{2.2}, R^{2.3}, and/or R^{2.4}, the definition of R³ is assumed by R^{3.1}, R^{3.2}, R^{3.3}, and/or R^{3.4}, the definition of R⁴ is assumed by R^{4.1}, R^{4.2}, R^{4.3}, and/or R^{4.4}, the definition of R⁵ is assumed by R^{5.1}, R^{5.2}, R^{5.3}, and/or R^{5.4}, the definition of R⁶ is assumed by R^{6.1}, R^{6.2}, R^{6.3}, and/or R^{6.4}, the definition of R⁷ is assumed by R^{7.1}, R^{7.2}, R^{7.3}, and/or R^{7.4}, the definition of R⁹ is assumed by R^{9.1}, R^{9.2}, R^{9.3}, and/or R^{9.4}, the definition of R¹⁰ is assumed by R^{10.1}, R^{10.2}, R^{10.3}, and/or R^{10.4}, the definition of RH is assumed by R^{11.1}, R^{11.2}, R^{11.3}, and/or R^{11.4}, the definition of R¹² is assumed by R^{12.1}, R^{12.2}, R^{12.3}, and/or R^{12.4}, the definition of R¹³ is assumed by R^{13.1}, R^{13.2}, R^{13.3}, and/or R^{13.4}, the definition of R¹⁴ is assumed by R^{14.1}, R^{14.2}, R^{14.3}, and/or R^{14.4}. The variables used within a definition of R¹, R², R³, R⁴, R⁵, R⁶, R⁷, R⁹, R¹⁰, R¹¹, R¹², R¹³ and/or R¹⁴, and/or other variables that appear at multiple instances and are different may similarly be appropriately labeled to distinguish each group for greater clarity.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

### Humanized Antibodies

A humanized antibody is a genetically engineered antibody in which at least one CDR (or functional fragment thereof) from a mouse antibody (“donor antibody”, which can also be rat, hamster or other non-human species) are grafted onto a human antibody (“acceptor antibody”). The human antibody is a non-natural (e.g. not naturally occurring or not naturally produced by a human) antibody that does not elicit an immune response in a human, does not elicit a significant immune response in a human, or elicits an immune response that is less than the immune response elicited in a mouse. In embodiments, more than one mouse CDR is grafted (e.g. all six mouse CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, a humanized antibody may be an antibody having one or more CDRs from a donor antibody and a variable region framework (FR). The FR may form part of a constant region and/or a variable region within a human antibody. In addition, in order to retain high binding affinity, amino acids in the human acceptor sequence may be replaced by the corresponding amino acids from the donor sequence, for example where: (1) the amino acid is in a CDR; (2) the amino acid is in the human framework region (e.g. the amino acid is immediately adjacent to one of the CDR's). See, U.S. Pat. Nos. 5,530,101 and 5,585,089, incorporated herein by reference, which provide detailed instructions for construction of humanized antibodies. Although humanized antibodies often incorporate all six CDRs (e.g. as defined by Kabat, but often also including hypervariable loop H1 as defined by Chothia) from a mouse antibody, they can also be made with fewer mouse CDRs and/or less than the complete mouse CDR sequence (e.g. a functional fragment of a CDR) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

Typically a humanized antibody as provided herein may include (i) a light chain comprising at least one CDR (often three CDRs) from a mouse antibody (also referred to herein as a mouse CDR) and a human variable region framework; and (ii) a heavy chain comprising at least one CDR (often three CDRs) from the mouse antibody and a human variable region framework (FR). The light and heavy chain variable region frameworks (FRs) may each be a mature human antibody variable region framework sequence (or fragment thereof), a germline variable region framework sequence (combined with a J region sequence) (or fragment thereof), or a consensus sequence of a human antibody variable region framework sequence (or fragment thereof). In embodiments, the humanized antibody includes a light chain as described in (i), a heavy chain as described in (ii) together with a light chain human constant region and a heavy chain constant region.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or using transgenic animals (Lonberg et al., WO93/12227; Kucherlapati WO91/10741, each of which is incorporated herein by reference).

Other approaches to design humanized antibodies may also be used to achieve the same result as the methods in U.S. Pat. Nos. 5,530,101 and 5,585,089 described above, for example, “superhumanization” (see Tan et al. J. Immunol. 169: 1119, 2002, and U.S. Pat. No. 6,881,557) or the method of Studnicak et al., Protein Eng. 7:805, 1994. Moreover, other approaches to produce genetically engineered, reduced-immunogenicity mAbs include “reshaping”, “hyperchimerization” and veneering/resurfacing, as described, e.g., in Vaswami et al., Annals of Allergy, Asthma and Immunology 81:105, 1998; Roguska et al. Protein Eng. 9:895, 1996; and U.S. Pat. Nos. 6,072,035 and 5,639,641.

A “CD73 protein” or “CD73 antigen” as referred to herein includes any of the recombinant or naturally-occurring forms of the Cluster of Differentiation 73 (CD73) also known as 5′-nucleotidase (5′-NT) or ecto-5′-nucleotidase or variants or homologs thereof that maintain CD73 nucleotidase activity (e.g. within at least 50%, 80%, 90%, 95%, 96%, 97%, 98%, 99% or 100% activity compared to CD73). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD73 protein. In embodiments, the CD73 protein is substantially identical to the protein identified by the UniProt reference number 21589 or a variant or homolog having substantial identity thereto. In embodiments, the CD73 protein is substantially identical to the protein identified by the UniProt reference number Q61503 or a variant or homolog having substantial identity thereto.

### Methods of Treating CD73 Expressing Antibodies

Provided herein are, inter alia, methods of treating cancer by administering to a subject a therapeutically effective amount of an antibody capable of binding CD73 (e.g., an 1E9 antibody, IgG1 antibody or humanized 1E9 antibody). The antibodies used for the methods provided herein including embodiments thereof are capable of binding CD73 proteins thereby inhibiting their catalytic activity and preventing metastasis. The methods provided herein are particularly useful to treat subjects which express an elevated level of CD73 relative to a standard control. “An elevated level of CD73” as referred to herein is a level of CD73 expressed by a tumor in a subject. A “tumor” as provided herein is a malignant or benign cellular mass including cancer cells and non-cancer cells. The non-cancer cells forming part of a tumor may be, for example, stromal cells or immune cells (e.g., T cells, dendritic cells, B cells, macrophages). Thus, the elevated level of CD73 may be expressed by a non-cancer cell (e.g., a stromal cell) forming part of a tumor or a cancer cell (e.g., a malignant T cell) forming part of a tumor. In embodiments, the elevated level of CD73 is expressed by a non-cancer cell. In embodiments, the elevated level of CD73 is expressed by a stromal cell.

The methods provided herein are particularly useful for the treatment of cancer in subjects which express an elevated level of CD73 relative to a standard control. A “standard control” as referred to herein refers to a sample that serves as a reference, usually a known reference, for comparison to a test sample. For example, a test sample can be taken from a patient suspected of having cancer and compared to samples from a known cancer patient, or a known normal (non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. In some examples of the disclosed methods, when the expression level of CD73 is assessed, the level is compared with a control expression level of CD73. By control expression level is meant the expression level of CD73 from a sample or subject lacking cancer, a sample or subject at a selected stage of cancer or cancer state, or in the absence of a particular variable such as a therapeutic agent. Alternatively, the control level comprises a known amount of CD73. Such a known amount correlates with an average level of subjects lacking cancer, at a selected stage of cancer or cancer state, or in the absence of a particular variable such as a therapeutic agent. A control level also includes the expression level of CD73 from one or more selected samples or subjects as described herein. For example, a control level includes an assessment of the expression level of CD73 in a sample from a subject that does not have cancer, is at a selected stage of cancer or cancer state, or has not received treatment for cancer. Another exemplary control level includes an assessment of the expression level of CD73 in samples taken from multiple subjects that do not have cancer, are at a selected stage of cancer, or have not received treatment for cancer.

When the control level includes the expression level of CD73 in a sample or subject in the absence of a therapeutic agent, the control sample or subject is optionally the same sample or subject to be tested before or after treatment with a therapeutic agent or is a selected sample or subject in the absence of the therapeutic agent. Alternatively, a control level is an average expression level calculated from a number of subjects without a particular disease. A control level also includes a known control level or value known in the art.

In one aspect, a method of treating cancer in a subject in need thereof is provided. The method includes administering to the subject a therapeutically effective amount of a 1E9 antibody, wherein the subject expresses an elevated level of CD73 relative to a standard control, and wherein the 1E9 antibody includes (i) a mouse CDR L1 as set forth in SEQ ID NO:1, a mouse CDR L2 as set forth in SEQ ID NO:2, a mouse CDR L3 as set forth in SEQ ID NO:3; and (ii) a mouse CDR H1 as set forth in SEQ ID NO:4, a mouse CDR H2 as set forth in SEQ ID NO:5, and a mouse CDR H3 as set forth in SEQ ID NO:6.

The term “therapeutically effective amount,” as used herein, refers to that amount of antibody (e.g., an anti-CD73 antibody, an 1E9 antibody, IgG1 antibody, IgG4 antibody or humanized 1E9 antibody) sufficient to ameliorate the disorder (e.g., cancer), as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. Toxicity and therapeutic efficacy of antibodies can be determined by standard pharmaceutical procedures in cell cultures or experimental animals, e.g., by determining the LD50 (the dose lethal to 50% of the population), the ED50 (the dose therapeutically effective in 50% of the population). The dose ratio of toxic to therapeutic effects is the therapeutic index, which can be expressed as the ratio LD50/ED50.

In embodiments, the 1E9 antibody is a humanized 1E9 antibody. In embodiments, the humanized 1E9 antibody includes a humanized light chain variable region and humanized heavy chain variable region, wherein the humanized light chain variable region includes a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87, and the humanized heavy chain variable region includes an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized light chain variable region provided herein includes a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87.

In embodiments, the humanized light chain variable region includes a valine at a position corresponding to Kabat position 2. In embodiments, the humanized light chain variable region includes a methionine at a position corresponding to Kabat position 4. In embodiments, the humanized light chain variable region an aspartic acid or a leucine at a position corresponding to Kabat position 9. In embodiments, the humanized light chain variable region includes a proline or a serine at a position corresponding to Kabat position 12. In embodiments, the humanized light chain variable region includes a lysine or a proline at a position corresponding to Kabat position 18. In embodiments, the humanized light chain variable region includes an alanine at a position corresponding to Kabat position 43. In embodiments, the humanized light chain variable region includes a proline or a serine at a position corresponding to Kabat position 60.

In embodiments, the humanized light chain variable region includes a threonine at a position corresponding to Kabat position 74. In embodiments, the humanized light chain variable region includes an asparagine or a serine at a position corresponding to Kabat position 76. In embodiments, the humanized light chain variable region includes an asparagine or a serine at a position corresponding to Kabat position 77. In embodiments, the humanized light chain variable region includes an isoleucine or a leucine at a position corresponding to Kabat position 78. In embodiments, the humanized light chain variable region includes a serine or an alanine at a position corresponding to Kabat position 80. In embodiments, the humanized light chain variable region includes a glutamine at a position corresponding to Kabat position 100. In embodiments, the humanized light chain variable region includes a valine at a position corresponding to Kabat position 104. In embodiments, the humanized light chain variable region includes a glutamic acid or an alanine at a position corresponding to Kabat position 1. In embodiments, the humanized light chain variable region includes a glutamine at a position corresponding to Kabat position 3.

In embodiments, the humanized light chain variable region includes a phenylalanine or a threonine at a position corresponding to Kabat position 10. In embodiments, the humanized light chain variable region includes a glutamine at a position corresponding to Kabat position 11. In embodiments, the humanized light chain variable region includes an alanine or a leucine at a position corresponding to Kabat position 13. In embodiments, the humanized light chain variable region includes a threonine at a position corresponding to Kabat position 14. In embodiments, the humanized light chain variable region includes a valine or a proline at a position corresponding to Kabat position 15. In embodiments, the humanized light chain variable region includes a lysine at a position corresponding to Kabat position 16. In embodiments, the humanized light chain variable region includes a glutamic acid or an aspartic acid at a position corresponding to Kabat position 17. In embodiments, the humanized light chain variable region includes a threonine at a position corresponding to Kabat position 22.

In embodiments, the humanized light chain variable region includes a lysine at a position corresponding to Kabat position 42. In embodiments, the humanized light chain variable region includes an arginine at a position corresponding to Kabat position 45. In embodiments, the humanized light chain variable region includes a an isoleucine at a position corresponding to Kabat position 58. In embodiments, the humanized light chain variable region includes a tyrosine at a position corresponding to Kabat position 67. In embodiments, the humanized light chain variable region includes a phenylalanine at a position corresponding to Kabat position 73. In embodiments, the humanized light chain variable region includes an isoleucine at a position corresponding to Kabat position 78. In embodiments, the humanized light chain variable region includes a tyrosine at a position corresponding to Kabat position 85. In embodiments, the humanized light chain variable region includes a phenylalanine at a position corresponding to Kabat position 87.

The humanized heavy chain variable region provided herein may include an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized heavy chain variable region includes an isoleucine at a position corresponding to Kabat position 37. In embodiments, the humanized heavy chain variable region includes an alanine or a proline at a position corresponding to Kabat position 40. In embodiments, the humanized heavy chain variable region includes a lysine at a position corresponding to Kabat position 43. In embodiments, the humanized heavy chain variable region includes a serine at a position corresponding to Kabat position 70. In embodiments, the humanized heavy chain variable region includes an isoleucine or a threonine at a position corresponding to Kabat position 75. In embodiments, the humanized heavy chain variable region includes a tryptophan at a position corresponding to Kabat position 82. In embodiments, the humanized heavy chain variable region includes an arginine or a lysine at a position corresponding to Kabat position 83. In embodiments, the humanized heavy chain variable region includes an alanine at a position corresponding to Kabat position 84.

In embodiments, the humanized heavy chain variable region includes a serine at a position corresponding to Kabat position 85. In embodiments, the humanized heavy chain variable region includes a valine or a methionine at a position corresponding to Kabat position 89. In embodiments, the humanized heavy chain variable region includes a valine at a position corresponding to Kabat position 5. In embodiments, the humanized heavy chain variable region includes a serine at a position corresponding to Kabat position 7. In embodiments, the humanized heavy chain variable region includes a valine at a position corresponding to Kabat position 11. In embodiments, the humanized heavy chain variable region includes a glutamic acid or a lysine at a position corresponding to Kabat position 12. In embodiments, the humanized heavy chain variable region includes an isoleucine or a valine at a position corresponding to Kabat position 20. In embodiments, the humanized heavy chain variable region includes an arginine at a position corresponding to Kabat position 38. In embodiments, the humanized heavy chain variable region includes an arginine at a position corresponding to Kabat position 66. In embodiments, the humanized heavy chain variable region includes a valine at a position corresponding to Kabat position 67.

In embodiments, the humanized heavy chain variable region includes an isoleucine at a position corresponding to Kabat position 69. In embodiments, the humanized heavy chain variable region includes an alanine at a position corresponding to Kabat position 71. In embodiments, the humanized heavy chain variable region includes a lysine at a position corresponding to Kabat position 73. In embodiments, the humanized heavy chain variable region includes a threonine at a position corresponding to Kabat position 87. In embodiments, the humanized heavy chain variable region includes a glutamic acid at a position corresponding to Kabat position 1. In embodiments, the humanized heavy chain variable region includes a valine at a position corresponding to Kabat position 24. In embodiments, the humanized heavy chain variable region includes an arginine at a position corresponding to Kabat position 44. In embodiments, the humanized heavy chain variable region includes a methionine at a position corresponding to Kabat position 48. In embodiments, the humanized heavy chain variable region includes a leucine at a position corresponding to Kabat position 80. In embodiments, the humanized heavy chain variable region includes a glutamic acid at a position corresponding to Kabat position 81.

The elevated level of CD73 may be determined using standard methods commonly known in the art. For example, the elevated level of CD73 may be determined by determining an H-score for said elevated level of CD73. Thus, the elevated level of CD73 may be defined by an H-score. As used herein, an “H-score” or “Histoscore” is a numerical value determined by a semi-quantitative method commonly known for immunohistochemically evaluating protein expression (e.g., CD73 expression) in tumor samples. The H-score may be calculated using the following formula:

[1×(% cells 1+)+2×(% cells 2+)+3×(% cells 3+)].

According to this formula, the H-score is calculated by determining the percentage of cells having a given staining intensity level (e.g., a CD73 staining intensity level) (i.e., level 1+, 2+, or 3+ from lowest to highest intensity level), weighting the percentage of cells having the given intensity level by multiplying the cell percentage by a factor (e.g., 1, 2, or 3) that gives more relative weight to cells with higher-intensity membrane staining, and summing the results to obtain a H-score. Commonly H-scores range from 0 to 300. Further description on the determination of H-scores in tumor cells can be found in Hirsch F R, Varella-Garcia M, Bunn P A Jr., et al. (Epidermal growth factor receptor in non-small-cell lung carcinomas: Correlations between gene copy number and protein expression and impact on prognosis. J Clin Oncol 21: 3798-3807, 2003) and John T, Liu G, Tsao M-S(Overview of molecular testing in non-small-cell lung cancer: Mutational analysis, gene copy number, protein expression and other biomarkers of EGFR for the prediction of response to tyrosine kinase inhibitors. Oncogene 28:S14-S23, 2009), which are hereby incorporated by reference in their entirety and for all purposes. Immunohistochemistry or other methods known in the art may be used for detecting CD73 expression. In embodiments, the H-score (CD73 H-score) of a cancer cell is determined. In embodiments, the H-score (CD73 H-score) of a non-cancer cell is determined. In embodiments, the non-cancer cell is a stromal cell. In embodiments, the H-score of a cancer cell and a non-cancer cell is determined.

In embodiments, the elevated level of CD73 has an H-score of at least 150 (e.g., 155, 160, 165, 170, 175, 180, 185, 190, 195, 200, 205, 210, 215, 220, 230, 240, 250, 260, 270, 280, 290, 300). In embodiments, elevated level of CD73 has an H-score of at least 155. In embodiments, elevated level of CD73 has an H-score of at least 160. In embodiments, elevated level of CD73 has an H-score of at least 165. In embodiments, elevated level of CD73 has an H-score of at least 170. In embodiments, elevated level of CD73 has an H-score of at least 175. In embodiments, elevated level of CD73 has an H-score of at least 180. In embodiments, elevated level of CD73 has an H-score of at least 185. In embodiments, elevated level of CD73 has an H-score of at least 190. In embodiments, elevated level of CD73 has an H-score of at least 195. In embodiments, elevated level of CD73 has an H-score of at least 200. In embodiments, elevated level of CD73 has an H-score of at least 205. In embodiments, elevated level of CD73 has an H-score of at least 210. In embodiments, elevated level of CD73 has an H-score of at least 215. In embodiments, elevated level of CD73 has an H-score of at least 220. In embodiments, elevated level of CD73 has an H-score of at least 230. In embodiments, elevated level of CD73 has an H-score of at least 240. In embodiments, elevated level of CD73 has an H-score of at least 250. In embodiments, elevated level of CD73 has an H-score of at least 260. In embodiments, elevated level of CD73 has an H-score of at least 270. In embodiments, elevated level of CD73 has an H-score of at least 280. In embodiments, elevated level of CD73 has an H-score of at least 290. In embodiments, elevated level of CD73 has an H-score of 300.

In embodiments, elevated level of CD73 has an H-score of about 150. In embodiments, elevated level of CD73 has an H-score of about 155. In embodiments, elevated level of CD73 has an H-score of about 160. In embodiments, elevated level of CD73 has an H-score of about 165. In embodiments, elevated level of CD73 has an H-score of about 170. In embodiments, elevated level of CD73 has an H-score of about 175. In embodiments, elevated level of CD73 has an H-score of about 180. In embodiments, elevated level of CD73 has an H-score of about 185. In embodiments, elevated level of CD73 has an H-score of about 190. In embodiments, elevated level of CD73 has an H-score of about 195. In embodiments, elevated level of CD73 has an H-score of about 200. In embodiments, elevated level of CD73 has an H-score of about 205. In embodiments, elevated level of CD73 has an H-score of about 210. In embodiments, elevated level of CD73 has an H-score of about 215. In embodiments, elevated level of CD73 has an H-score of about 220. In embodiments, elevated level of CD73 has an H-score of about 230. In embodiments, elevated level of CD73 has an H-score of about 240. In embodiments, elevated level of CD73 has an H-score of about 250. In embodiments, elevated level of CD73 has an H-score of about 260. In embodiments, elevated level of CD73 has an H-score of about 270. In embodiments, elevated level of CD73 has an H-score of about 280. In embodiments, elevated level of CD73 has an H-score of about 290. In embodiments, elevated level of CD73 has an H-score of 300. In embodiments, the elevated level of CD73 has an H-score of about 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299 or 300.

The elevated level of CD73 may be determined by determining a stromal score for said elevated level of CD73. A “stromal score” as used herein, refers to the percentage of CD73 expressing stromal cells (e.g., non-tumor cells including, for example, fibroblasts, pericytes, endothelial cells, etc.) per tumor surface in a tissue sample. Immunohistochemistry or other methods known in the art may be used for detecting CD73 expression on stromal cells.

In embodiments, the elevated level of CD73 has a stromal score of at least 50% (e.g., 51, 52, 53, 54, 55, 56, 57, 58, 59, 60, 65, 70, 75, 80, 85, 90, 95, 100%). For example, a tissue sample has a stromal score of at least 50% when at least 50 stromal cells out of 100 tumor cells express CD73. In embodiments, the elevated level of CD73 has a stromal score of at least 51%. In embodiments, the elevated level of CD73 has a stromal score of at least 52%. In embodiments, the elevated level of CD73 has a stromal score of at least 53%. In embodiments, the elevated level of CD73 has a stromal score of at least 54%. In embodiments, the elevated level of CD73 has a stromal score of at least 55%. In embodiments, the elevated level of CD73 has a stromal score of at least 56%. In embodiments, the elevated level of CD73 has a stromal score of at least 57%. In embodiments, the elevated level of CD73 has a stromal score of at least 58%. In embodiments, the elevated level of CD73 has a stromal score of at least 59%. In embodiments, the elevated level of CD73 has a stromal score of at least 60%. In embodiments, the elevated level of CD73 has a stromal score of at least 65%. In embodiments, the elevated level of CD73 has a stromal score of at least 70%. In embodiments, the elevated level of CD73 has a stromal score of at least 75%. In embodiments, the elevated level of CD73 has a stromal score of at least 80%. In embodiments, the elevated level of CD73 has a stromal score of at least 85%. In embodiments, the elevated level of CD73 has a stromal score of at least 90%. In embodiments, the elevated level of CD73 has a stromal score of at least 95%. In embodiments, the elevated level of CD73 has a stromal score of 100%.

The antibodies provided herein, including embodiments thereof, may bind at or near the CD73 active site. In embodiments, a CD73 molecule is bound by a single antibody provided herein. In embodiments, the CD73 molecule is bound by more than one (at least two) antibody provided herein. Thus, the antibodies provided herein may form monovalent or multivalent interactions with a CD73 protein. In embodiments, the therapeutically effective amount of the antibody is administered at a 1:1 ratio relative to the elevated level of CD73.

In embodiments, the antibody is administered at a half maximal effective concentration (EC₅₀) of at least 100 nM (e.g., 110, 115, 120, 125, 130, 135, 140, 145, 150, 155, 160, 165, 170, 175, 180, 185, 190, 195, 200, 210, 220, 230, 240, 250 nM). In embodiments, the antibody is administered at a half maximal effective concentration (EC₅₀) of 110 nM. In embodiments, the antibody is administered at a half maximal effective concentration (EC₅₀) of 115 nM. In embodiments, the antibody is administered at a half maximal effective concentration (EC₅₀) of 120 nM. In embodiments, the antibody is administered at a half maximal effective concentration (EC₅₀) of 125 nM. In embodiments, the antibody is administered at a half maximal effective concentration (EC₅₀) of 130 nM. In embodiments, the antibody is administered at a half maximal effective concentration (EC₅₀) of 135 nM. In embodiments, the antibody is administered at a half maximal effective concentration (EC₅₀) of 140 nM. In embodiments, the antibody is administered at a half maximal effective concentration (EC₅₀) of 145 nM. In embodiments, the antibody is administered at a half maximal effective concentration (EC₅₀) of 150 nM. In embodiments, the antibody is administered at a half maximal effective concentration (EC₅₀) of 155 nM. In embodiments, the antibody is administered at a half maximal effective concentration (EC₅₀) of 160 nM. In embodiments, the antibody is administered at a half maximal effective concentration (EC₅₀) of 165 nM. In embodiments, the antibody is administered at a half maximal effective concentration (EC₅₀) of 170 nM. In embodiments, the antibody is administered at a half maximal effective concentration (EC₅₀) of 175 nM. In embodiments, the antibody is administered at a half maximal effective concentration (EC₅₀) of 180 nM. In embodiments, the antibody is administered at a half maximal effective concentration (EC₅₀) of 185 nM. In embodiments, the antibody is administered at a half maximal effective concentration (EC₅₀) of 190 nM. In embodiments, the antibody is administered at a half maximal effective concentration (EC₅₀) of 195 nM. In embodiments, the antibody is administered at a half maximal effective concentration (EC₅₀) of 200 nM. In embodiments, the antibody is administered at a half maximal effective concentration (EC₅₀) of 210 nM. In embodiments, the antibody is administered at a half maximal effective concentration (EC₅₀) of 220 nM. In embodiments, the antibody is administered at a half maximal effective concentration (EC₅₀) of 230 nM. In embodiments, the antibody is administered at a half maximal effective concentration (EC₅₀) of 240 nM. In embodiments, the antibody is administered at a half maximal effective concentration (EC₅₀) of 250 nM.

In embodiments, the antibody is administered at an EC₅₀ of about 137 nM. In embodiments, the antibody is administered at an EC₅₀ of 137 nM. In embodiments, the antibody is administered at an EC₅₀ of about 189 nM. In embodiments, the antibody is administered at an EC₅₀ of 189 nM.

In embodiments, the therapeutically effective amount is about 0.05 mg/kg, 0.1 mg/kg, 0.3 mg/kg, 3 mg/kg, 10 mg/kg, 30 mg/kg, 40 mg/kg, or 120 mg/kg. In embodiments, the therapeutically effective amount is about 0.05 mg/kg. In embodiments, the therapeutically effective amount is 0.05 mg/kg. In embodiments, the therapeutically effective amount is about 0.1 mg/kg. In embodiments, the therapeutically effective amount is 0.1 mg/kg. In embodiments, the therapeutically effective amount is about 0.3 mg/kg. In embodiments, the therapeutically effective amount is 0.3 mg/kg. In embodiments, the therapeutically effective amount is about 3 mg/kg. In embodiments, the therapeutically effective amount is 3 mg/kg. In embodiments, the therapeutically effective amount is about 10 mg/kg. In embodiments, the therapeutically effective amount is 10 mg/kg. In embodiments, the therapeutically effective amount is about 30 mg/kg. In embodiments, the therapeutically effective amount is 30 mg/kg. In embodiments, the therapeutically effective amount is about 40 mg/kg. In embodiments, the therapeutically effective amount is 40 mg/kg. In embodiments, the therapeutically effective amount is about 120 mg/kg. In embodiments, the therapeutically effective amount is 120 mg/kg. In embodiments, the effective amount administered results in serum levels of the antibody of about 10 μg/ml.

The humanized 1E9 antibodies as provided herein are capable of binding a CD73 protein and include at least one CDR or a functional fragment thereof of the mouse monoclonal antibody 1E9 (Thomson L F et al. Tissue Antigens 2008, Volume 35, Issue 1: Production and characterization of monoclonal antibodies to the glycosyl phosphatidylinositol-anchored lymphocyte differentiation antigen ecto-5′-nucleotidase (CD73)). A functional fragment of a CDR is a portion of a complete CDR amino acid sequence that is capable of binding to an antigen (e.g., CD73). Thus, a functional fragment of a CDR typically includes the amino acid residues required for CDR binding to the antigen (e.g., CD73). A “mouse CDR” is a complete CDR amino acid sequence or a functional fragment thereof derived from a mouse antibody that is capable of binding CD73. Thus, a functional fragment of a mouse CDR typically includes the amino acid residues required for CDR binding to CD73. Where a humanized 1E9 antibody includes at least one mouse CDR, the at least one mouse CDR or a functional fragment thereof is derived from a donor antibody. In embodiments, the donor antibody is a mouse 1E9 antibody. A person of skill in the art will immediately recognize that a humanized 1E9 antibody including at least one mouse CDR is a humanized antibody with at least one mouse CDR derived from a donor 1E9 antibody and the additional CDRs are derived from the acceptor antibody (e.g. where the light chain includes a total of three CDRs and the heavy chain includes a total of three CDRs).

In embodiments, the humanized light chain variable region and the humanized heavy chain variable region include combined one mouse CDR or functional fragment of a mouse CDR. Thus, in some embodiments, the humanized light chain variable region and the humanized heavy chain variable region include combined six CDRs wherein at least one of the six CDRs is a mouse CDR. Where the humanized light chain variable region and the humanized heavy chain variable region include combined one mouse CDR, the humanized light chain variable region or the humanized heavy chain variable region include one mouse CDR. For example, a humanized antibody may include CDR L3 derived from the donor antibody (e.g. mouse, also referred to herein as a mouse CDR L3) and CDR L1, CDR L2, CDR H1, CDR H2, and CDR H3 derived from the acceptor antibody (i.e. human).

In embodiments, the humanized light chain variable region and the humanized heavy chain variable region include combined two mouse CDRs. Where the humanized light chain variable region and the humanized heavy chain variable region include combined two mouse CDRs, the humanized light chain variable region and the humanized heavy chain variable region each include one mouse CDR (i), the humanized light chain variable region includes two mouse CDRs (ii), or the humanized heavy chain variable region includes two mouse CDRs (iii). For example, a humanized antibody may include CDR L3 and CDR H3 derived from the donor antibody (also referred to herein as a mouse CDR L3 and a mouse CDR H3, respectively), and CDR L1, CDR L2, CDR H1, and CDR H2 derived from the acceptor antibody (i.e. human).

In embodiments, the humanized light chain variable region and the humanized heavy chain variable region include combined three mouse CDRs. Where the humanized light chain variable region and the humanized heavy chain variable region include combined three mouse CDRs, the humanized light chain variable region may include one mouse CDR and the humanized heavy chain variable region may include two mouse CDRs (i), the humanized light chain variable region includes two mouse CDRs and the humanized heavy chain variable region includes one mouse CDR (ii), the humanized light chain variable region includes three mouse CDRs (iii), or the humanized heavy chain variable region includes three mouse CDRs (iv). For example, a humanized antibody may include CDR L3, CDR H3 and CDR L2 derived from the donor antibody (e.g. mouse, also referred to herein as a CDR L3, mouse CDR H3, and mouse CDR L2 respectively) and CDR L1, CDR H1, and CDR H2 derived from the acceptor antibody (i.e. human).

In embodiments, the humanized light chain variable region and the humanized heavy chain variable region include combined four mouse CDRs. Where the humanized light chain variable region and the humanized heavy chain variable region include combined four mouse CDRs, the humanized light chain variable region includes one mouse CDR and the humanized heavy chain variable region includes three mouse CDRs (i), the humanized light chain variable region includes three mouse CDRs and the humanized heavy chain variable region includes one mouse CDR (ii), or the humanized light chain variable region includes two mouse CDRs and the humanized heavy chain variable region includes two mouse CDRs (iii). For example, a humanized antibody may include CDR L3, CDR H3, CDR L2 and CDR L1 derived from the donor antibody (e.g. mouse, also referred to herein as a mouse CDR L3, mouse CDR H3, mouse CDR L2 and mouse CDR L1 respectively) and CDR H1 and CDR H2 derived from the acceptor antibody (i.e. human).

In embodiments, the humanized light chain variable region and the humanized heavy chain variable region each include at least one mouse CDR. Where the humanized light chain variable region and the humanized heavy chain variable region each include at least one mouse CDR, the humanized light chain variable region includes at least one mouse CDR and the humanized heavy chain variable region includes at least one mouse CDR. Thus, in some embodiments, the humanized light chain variable region includes mouse CDR L1 and the humanized heavy chain includes mouse CDR H1. In embodiments, mouse CDR L1 includes the amino acid sequence of SEQ ID NO:1 and mouse CDR H1 includes the amino acid sequence of SEQ ID NO:4. In embodiments, mouse CDR L1 is the amino acid sequence of SEQ ID NO:1 and mouse CDR H1 is the amino acid sequence of SEQ ID NO:4. In embodiments, the humanized light chain variable region includes mouse CDR L2 and the humanized heavy chain variable region includes mouse CDR H2. In embodiments, mouse CDR L2 includes the amino acid sequence of SEQ ID NO:2 and mouse CDR H2 includes the amino acid sequence of SEQ ID NO:5. In embodiments, mouse CDR L2 is the amino acid sequence of SEQ ID NO:2 and mouse CDR H2 is the amino acid sequence of SEQ ID NO:5. In embodiments, the humanized light chain variable region includes mouse CDR L3 and the humanized heavy chain variable region includes mouse CDR H3. In embodiments, mouse CDR L3 includes the amino acid sequence of SEQ ID NO:3 and mouse CDR H3 includes the amino acid sequence of SEQ ID NO:6. In embodiments, CDR L3 is the amino acid sequence of SEQ ID NO:3 and mouse CDR H3 is the amino acid sequence of SEQ ID NO:6.

In embodiments, the presence of mouse CDR L3 and mouse CDR H3 may be sufficient for binding of a humanized antibody to CD73. Thus, in embodiments, the humanized antibody does not include mouse CDR L1, mouse CDR L2, CDR H1 or mouse CDR H2. Where the humanized antibody does not include mouse CDR L1, mouse CDR L2, mouse CDR H1 or mouse CDR H2, the humanized antibody includes CDR L1, CDR L2, CDR H1 or CDR H2 derived from the acceptor antibody (i.e. human). Thus, a humanized antibody that does not include mouse CDR L1, mouse CDR L2, mouse CDR H1 or mouse CDR H2, does not include CDR L1, CDR L2, CDR H1 or CDR H2 from a donor antibody (e.g. mouse, rat, rabbit), but includes CDR L1, CDR L2, CDR H1 or CDR H2 from the acceptor antibody (i.e. human). Thus, in embodiments the humanized light chain variable region does not include mouse CDR L1 or mouse CDR L2 and the humanized heavy chain variable region does not include mouse CDR H1 or mouse CDR H2. In embodiments, the humanized light chain variable region does not include mouse CDR L1 and mouse CDR L2 and the humanized heavy chain variable region does not include mouse CDR H1 and mouse CDR H2.

In embodiments, the humanized light chain variable region includes mouse CDR L2 and mouse CDR L3 and the humanized heavy chain variable region includes mouse CDR H2 and mouse CDR H3. In embodiments, the humanized light chain variable region includes mouse CDR L1, mouse CDR L2 and mouse CDR L3 and the humanized heavy chain variable region includes mouse CDR H1, mouse CDR H2 and mouse CDR H3. In embodiments, the humanized light chain variable region includes mouse CDR L1 as set forth in SEQ ID NO:1, mouse CDR L2 as set forth in SEQ ID NO:2 and mouse CDR L3 as set forth in SEQ ID NO:3, and the humanized heavy chain variable region includes mouse CDR H1 as set forth in SEQ ID NO:4, mouse CDR H2 as set forth in SEQ ID NO:5, and mouse CDR H3 as set forth in SEQ ID NO:6.

The position of CDRs and FRs may be defined by the Kabat numbering system (Kabat et al., Sequences of Proteins of Immunological Interest, Fifth Edition, U.S. Department of Health and Human Services, U.S. Government Printing Office (1991)). Likewise, the positions occupied by individual residues within the light or the heavy chain of an antibody may be defined by the Kabat numbering system. Therefore, the location of residues required for binding within a humanized light chain and a humanized heavy chain of a humanized antibody may be defined by the position of the residue according to the Kabat numbering system as is well known in the art. As described above, a humanized antibody may be an antibody having CDRs from a donor antibody (e.g. mouse) and variable region framework (FR) from a human antibody. The framework regions (FRs) are said to hold the CDRs in place in a humanized antibody. Proceeding from the amino-terminus, these regions are designated FR L1, FR L2, FR L3, and FR L4 for the light chain and FR H1, FR H2, FR H3, and FR H4, for the heavy chain, respectively. Surprisingly, the present invention provides for humanized antibodies that include one or more residues within the framework regions that are important for epitope binding of the humanized antibody. A framework region residue involved in (or important for) epitope binding (e.g. CD73 binding) is referred to herein as a binding framework region residue. The binding framework region residues may reside in the framework region of a humanized light chain variable region (i.e. FR L1, FR L2, FR L3, FR L4) or they may reside in the framework of a humanized heavy chain variable region (i.e. FR H1, FR H2, FR H3, FR H4). A binding framework residue residing in the FR L3 region of a humanized light chain is referred to herein as a FR L3 binding framework region residue. Thus, a binding framework region residue residing in the FR H3 region of a humanized heavy chain is referred to herein as a FR H3 binding framework region residue.

In embodiments, the humanized antibody includes at least one binding framework region residue. In embodiments, the humanized light chain variable region includes at least one binding framework region residue. In embodiments, the humanized light chain variable region includes one or more FR L1, FR L2, FR L3 or FR L4 binding framework region residues. In embodiments, the humanized light chain variable region includes one or more FR L1 binding framework region residues. In embodiments, the humanized light chain variable region includes one or more FR L2 binding framework region residues. In embodiments, the humanized light chain variable region includes one or more FR L3 binding framework region residues. In embodiments, the humanized light chain variable region includes one or more FR L4 binding framework region residues. In embodiments, the humanized heavy chain variable region includes one or more FR H1, FR H2, FR H3 or FR H4 binding framework region residues. In embodiments, the humanized heavy chain variable region includes one or more FR H1 binding framework region residues. In embodiments, the humanized heavy chain variable region includes one or more FR H2 binding framework region residues. In embodiments, the humanized heavy chain variable region includes one or more FR H3 binding framework region residues. In embodiments, the humanized heavy chain variable region includes one or more FR H4 binding framework region residues.

In embodiments, the humanized light chain variable region includes at least one binding framework region residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42 43, 44, 45, 46, 47, 48, 49, 50 or more residues) and the humanized heavy chain variable region includes at least one binding framework region residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42 43, 44, 45, 46, 47, 48, 49, 50 or more residues). The position of a binding framework region residue within a humanized antibody may be defined by the Kabat numbering system similar to the positions CDR residues.

In one aspect, a method of treating cancer in a subject in need thereof is provided. The method includes administering to the subject a therapeutically effective amount of a humanized 1E9 antibody, wherein the subject expresses an elevated level of CD73 relative to a standard control and wherein the humanized 1E9 antibody includes a humanized light chain variable region including a mouse CDR L1, mouse CDR L2, or mouse CDR L3 and a humanized heavy chain variable region including a mouse CDR H1, mouse CDR H2, or mouse CDR H3. The humanized light chain variable region may include a mouse CDR L1 as set forth in SEQ ID NO:1, a mouse CDR L2 as set forth in SEQ ID NO:2, or a mouse CDR L3 as set forth in SEQ ID NO:3. The humanized light chain variable region may include a mouse CDR L1 as set forth in SEQ ID NO:1, a mouse CDR L2 as set forth in SEQ ID NO:2, and a mouse CDR L3 as set forth in SEQ ID NO:3. The humanized heavy chain variable region may include a mouse CDR H1 as set forth in SEQ ID NO:4, a mouse CDR H2 as set forth in SEQ ID NO:5, or a mouse CDR H3 as set forth in SEQ ID NO:6. The humanized heavy chain variable region may include a mouse CDR H1 as set forth in SEQ ID NO:4, a mouse CDR H2 as set forth in SEQ ID NO:5, and a mouse CDR H3 as set forth in SEQ ID NO:6. In embodiments, the humanized light chain variable region includes a mouse CDR L1 as set forth in SEQ ID NO:1. In embodiments, the humanized light chain variable region includes a mouse CDR L2 as set forth in SEQ ID NO:2. In embodiments, the humanized light chain variable region includes a mouse CDR L3 as set forth in SEQ ID NO:3. In embodiments, the humanized heavy chain variable region includes a mouse CDR H1 as set forth in SEQ ID NO:4. In embodiments, the humanized heavy chain variable region includes a mouse CDR H2 as set forth in SEQ ID NO:5. In embodiments, the humanized light chain variable region includes a mouse CDR H3 as set forth in SEQ ID NO:6. In further embodiments, the humanized light chain variable region includes at least one binding framework region residue. In other further embodiments, the humanized heavy chain variable region includes at least one binding framework region residue.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104, and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104, and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18, and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, and a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, and a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12 and a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85 and a methionine at a position corresponding to Kabat position 89.

In embodiments, the humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87, and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, the humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87, and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, the humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87, and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, the humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87, and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

In embodiments, the humanized heavy chain variable region includes the sequence of SEQ ID NO:7. In embodiments, the humanized heavy chain variable region is the sequence of SEQ ID NO:7. In embodiments, the humanized light chain variable region includes the sequence of SEQ ID NO:37. In embodiments, the humanized light chain variable region is the sequence of SEQ ID NO:37.

In embodiments, the humanized heavy chain variable region includes the sequence of SEQ ID NO:53. In embodiments, the humanized heavy chain variable region is SEQ ID NO:53. In embodiments, the humanized light chain variable region includes the sequence of SEQ ID NO:55. In embodiments, the humanized light chain variable region is SEQ ID NO:55. Thus, in another aspect, provided is a humanized 1E9 antibody including a humanized light chain variable region and a humanized heavy chain variable region, wherein the humanized heavy chain variable region includes the sequence of SEQ ID NO:53 and the humanized light chain variable region includes the sequence of SEQ ID NO:55.

In another aspect, a method of treating cancer in a subject in need thereof is provided. The method includes administering to the subject a therapeutically effective amount of a humanized 1E9 antibody, wherein the subject expresses an elevated level of CD73 relative to a standard control and wherein the humanized 1E9 antibody includes a humanized light chain variable region and a humanized heavy chain variable region, wherein the humanized heavy chain variable region includes the sequence of SEQ ID NO:7. In embodiments, the humanized light chain variable region includes the sequence of SEQ ID NO:37.

In embodiments, the antibody is an IgG. In embodiments, the antibody is an IgG1. In embodiments, the antibody is an IgG4. In embodiments, the antibody is an IgA. In embodiments, the antibody is an IgM.

The humanized 1E9 antibodies as provided herein may be Fab′ fragments. Where the humanized 1E9 antibodies are Fab′ fragments, the humanized 1E9 antibodies include a humanized heavy chain (e.g. including a constant and a variable region) and a humanized light chain (e.g. including a constant and a variable region). In embodiments, the antibody is a Fab′ fragment (e.g., anti-CD73 antibody, 1E9 antibody, humanized 1E9 antibody, IgG1 antibody, IgG4 antibody). In embodiments, the antibody includes a human constant region.

In embodiments, the antibody is a single chain antibody (scFv). A single chain antibody includes a variable light chain and a variable heavy chain. A person of skill in the art will immediately recognize that a single chain antibody includes a single light chain and a single heavy chain, in contrast to an immunoglobulin antibody, which includes two identical pairs of polypeptide chains, each pair having one light chain and one heavy chain. Each light chain and heavy chain in turn consists of two regions: a variable (“V”) region (i.e. variable light chain and variable heavy chain) involved in binding the target antigen, and a constant (“C”) region that interacts with other components of the immune system. The variable light chain and the variable heavy chain in a single chain antibody may be linked through a linker peptide. Examples for linker peptides of single chain antibodies are described in Bird, R. E., Hardman, K. D., Jacobson, J. W., Johnson, S., Kaufman, B. M., Lee, S. M., Lee, T., Pope, S. H., Riordan, G. S. and Whitlow, M. (1988). Methods of making scFv antibodies have been described. See, Huse et al., Science 246:1275-1281 (1989); Ward et al., Nature 341:544-546 (1989); and Vaughan et al., Nature Biotech. 14:309-314 (1996). Briefly, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell.

The ability of an antibody to bind a specific epitope (e.g., CD73) can be described by the equilibrium dissociation constant (K_{D}). The equilibrium dissociation constant (K_{D}) as defined herein is the ratio of the dissociation rate (K-off) and the association rate (K-on) of an antibody (e.g., anti-CD73 antibody, 1E9 antibody, humanized 1E9 antibody, IgG1 antibody, IgG4 antibody) or fragment thereof to a CD73 protein. It is described by the following formula: K_{D}=K-off/K-on. In embodiments, the antibody fragment provided herein (e.g., anti-CD73 antibody, 1E9 antibody, humanized 1E9 antibody, IgG1 antibody, IgG4 antibody) is capable of binding a CD73 antigen with an equilibrium dissociation constant (K_{D}) from about 0.3 to about 25 nM. In embodiments, the antibody is capable of binding a CD73 antigen with an equilibrium dissociation constant (K_{D}) from about 0.5 to about 25 nM. In embodiments, the antibody is capable of binding a CD73 antigen with an equilibrium dissociation constant (K_{D}) from about 1 to about 25 nM. In embodiments, the antibody is capable of binding a CD73 antigen with an equilibrium dissociation constant (K_{D}) from about 1.5 to about 25 nM. In embodiments, the antibody is capable of binding a CD73 antigen with an equilibrium dissociation constant (K_{D}) from about 2 to about 25 nM. In embodiments, the antibody is capable of binding a CD73 antigen with an equilibrium dissociation constant (K_{D}) from about 2.5 to about 25 nM. In embodiments, the antibody is capable of binding a CD73 antigen with an equilibrium dissociation constant (K_{D}) from about 3 to about 25 nM. In embodiments, the antibody is capable of binding a CD73 antigen with an equilibrium dissociation constant (K_{D}) from about 3.5 to about 25 nM. In embodiments, the antibody is capable of binding a CD73 antigen with an equilibrium dissociation constant (K_{D}) from about 4 to about 25 nM. In embodiments, the antibody is capable of binding a CD73 antigen with an equilibrium dissociation constant (K_{D}) in this paragraph at a pH below 7.5. In embodiments, the antibody is capable of binding a CD73 antigen with an equilibrium dissociation constant (K_{D}) in this paragraph at a pH of less than about 7.5. In embodiments, the antibody is capable of binding a CD73 antigen with an equilibrium dissociation constant (K_{D}) in this paragraph at a pH of less than about 7.0. In embodiments, the antibody is capable of binding a CD73 antigen with an equilibrium dissociation constant (K_{D}) in this paragraph at a pH of less than about 6.5. In embodiments, the antibody is capable of binding a CD73 antigen with an equilibrium dissociation constant (K_{D}) in this paragraph at a pH of less than about 6.0. In embodiments, the antibody is capable of binding a CD73 antigen with an equilibrium dissociation constant (K_{D}) in this paragraph at a pH of less than about 5.5. In embodiments, the antibody is capable of binding a CD73 antigen with an equilibrium dissociation constant (K_{D}) in this paragraph at a pH of less than about 5. In embodiments, the antibody is capable of binding a CD73 antigen with an equilibrium dissociation constant (K_{D}) in this paragraph at a pH of less than about 4.5. In embodiments, the antibody is capable of binding a CD73 antigen with an equilibrium dissociation constant (K_{D}) in this paragraph at a pH from about 6.0 to about 7.0. In embodiments, the antibody is capable of binding a CD73 antigen with an equilibrium dissociation constant (K_{D}) in this paragraph at a pH of about 6.0. In embodiments, the antibody is capable of binding a CD73 antigen with an equilibrium dissociation constant (K_{D}) in this paragraph at a pH of about 6.1. In embodiments, the antibody is capable of binding a CD73 antigen with an equilibrium dissociation constant (K_{D}) in this paragraph at a pH of about 6.2. In embodiments, the antibody is capable of binding a CD73 antigen with an equilibrium dissociation constant (K_{D}) in this paragraph at a pH of about 6.3. In embodiments, the antibody is capable of binding a CD73 antigen with an equilibrium dissociation constant (K_{D}) in this paragraph at a pH of about 6.4. In embodiments, the antibody is capable of binding a CD73 antigen with an equilibrium dissociation constant (K_{D}) in this paragraph at a pH of about 6.5. In embodiments, the antibody is capable of binding a CD73 antigen with an equilibrium dissociation constant (K_{D}) in this paragraph at a pH of about 6.6. In embodiments, the antibody is capable of binding a CD73 antigen with an equilibrium dissociation constant (K_{D}) in this paragraph at a pH of about 6.7. In embodiments, the antibody is capable of binding a CD73 antigen with an equilibrium dissociation constant (K_{D}) in this paragraph at a pH of about 6.8. In embodiments, the antibody is capable of binding a CD73 antigen with an equilibrium dissociation constant (K_{D}) in this paragraph at a pH of about 6.9. In embodiments, the antibody is capable of binding a CD73 antigen with an equilibrium dissociation constant (K_{D}) in this paragraph at a pH of about 7.0.

In embodiments, the antibody is capable of binding a CD73 antigen with an equilibrium dissociation constant (K_{D}) from about 4.5 to about 25 nM. In embodiments, the antibody is capable of binding a CD73 antigen with an equilibrium dissociation constant (K_{D}) from about 5 to about 25 nM. In embodiments, the antibody is capable of binding a CD73 antigen with an equilibrium dissociation constant (K_{D}) from about 5.5 to about 25 nM. In embodiments, the antibody is capable of binding a CD73 antigen with an equilibrium dissociation constant (K_{D}) from about 6 to about 25 nM. In embodiments, the antibody is capable of binding a CD73 antigen with an equilibrium dissociation constant (K_{D}) from about 6.5 to about 25 nM. In embodiments, the antibody is capable of binding a CD73 antigen with an equilibrium dissociation constant (K_{D}) from about 7 to about 25 nM. In embodiments, the antibody is capable of binding a CD73 antigen with an equilibrium dissociation constant (K_{D}) from about 7.5 to about 25 nM. In embodiments, the antibody is capable of binding a CD73 antigen with an equilibrium dissociation constant (K_{D}) from about 8 to about 25 nM. In embodiments, the antibody is capable of binding a CD73 antigen with an equilibrium dissociation constant (K_{D}) in this paragraph at a pH below 7.5. In embodiments, the antibody is capable of binding a CD73 antigen with an equilibrium dissociation constant (K_{D}) in this paragraph at a pH of less than about 7.5. In embodiments, the antibody is capable of binding a CD73 antigen with an equilibrium dissociation constant (K_{D}) in this paragraph at a pH of less than about 7.0. In embodiments, the antibody is capable of binding a CD73 antigen with an equilibrium dissociation constant (K_{D}) in this paragraph at a pH of less than about 6.5. In embodiments, the antibody is capable of binding a CD73 antigen with an equilibrium dissociation constant (K_{D}) in this paragraph at a pH of less than about 6.0. In embodiments, the antibody is capable of binding a CD73 antigen with an equilibrium dissociation constant (K_{D}) in this paragraph at a pH of less than about 5.5. In embodiments, the antibody is capable of binding a CD73 antigen with an equilibrium dissociation constant (K_{D}) in this paragraph at a pH of less than about 5. In embodiments, the antibody is capable of binding a CD73 antigen with an equilibrium dissociation constant (K_{D}) in this paragraph at a pH of less than about 4.5. In embodiments, the antibody is capable of binding a CD73 antigen with an equilibrium dissociation constant (K_{D}) in this paragraph at a pH from about 6.0 to about 7.0. In embodiments, the humanized antibody is capable of binding a CD73 antigen with an equilibrium dissociation constant (K_{D}) in this paragraph at a pH of about 6.0. In embodiments, the humanized antibody is capable of binding a CD73 antigen with an equilibrium dissociation constant (K_{D}) in this paragraph at a pH of about 6.1. In embodiments, the humanized antibody is capable of binding a CD73 antigen with an equilibrium dissociation constant (K_{D}) in this paragraph at a pH of about 6.2. In embodiments, the humanized antibody is capable of binding a CD73 antigen with an equilibrium dissociation constant (K_{D}) in this paragraph at a pH of about 6.3. In embodiments, the humanized antibody is capable of binding a CD73 antigen with an equilibrium dissociation constant (K_{D}) in this paragraph at a pH of about 6.4. In embodiments, the humanized antibody is capable of binding a CD73 antigen with an equilibrium dissociation constant (K_{D}) in this paragraph at a pH of about 6.5. In embodiments, the humanized antibody is capable of binding a CD73 antigen with an equilibrium dissociation constant (K_{D}) in this paragraph at a pH of about 6.6. In embodiments, the humanized antibody is capable of binding a CD73 antigen with an equilibrium dissociation constant (K_{D}) in this paragraph at a pH of about 6.7. In embodiments, the humanized antibody is capable of binding a CD73 antigen with an equilibrium dissociation constant (K_{D}) in this paragraph at a pH of about 6.8. In embodiments, the humanized antibody is capable of binding a CD73 antigen with an equilibrium dissociation constant (K_{D}) in this paragraph at a pH of about 6.9. In embodiments, the humanized antibody is capable of binding a CD73 antigen with an equilibrium dissociation constant (K_{D}) in this paragraph at a pH of about 7.0.

In embodiments, the humanized antibody is capable of binding a CD73 antigen with an equilibrium dissociation constant (K_{D}) from about 8.5 to about 25 nM. In embodiments, the humanized antibody is capable of binding a CD73 antigen with an equilibrium dissociation constant (K_{D}) from about 9 to about 25 nM. In embodiments, the humanized antibody is capable of binding a CD73 antigen with an equilibrium dissociation constant (K_{D}) from about 9.5 to about 25 nM. In embodiments, the humanized antibody is capable of binding a CD73 antigen with an equilibrium dissociation constant (K_{D}) from about 10 to about 25 nM. In embodiments, the humanized antibody is capable of binding a CD73 antigen with an equilibrium dissociation constant (K_{D}) from about 11 to about 25 nM. In embodiments, the humanized antibody is capable of binding a CD73 antigen with an equilibrium dissociation constant (K_{D}) from about 12 to about 25 nM. In embodiments, the humanized antibody is capable of binding a CD73 antigen with an equilibrium dissociation constant (K_{D}) from about 13 to about 25 nM. In embodiments, the humanized antibody is capable of binding a CD73 antigen with an equilibrium dissociation constant (K_{D}) from about 14 to about 25 nM. In embodiments, the humanized antibody is capable of binding a CD73 antigen with an equilibrium dissociation constant (K_{D}) from about 15 to about 25 nM. In embodiments, the humanized antibody is capable of binding a CD73 antigen with an equilibrium dissociation constant (K_{D}) from about 16 to about 25 nM. In embodiments, the humanized antibody is capable of binding a CD73 antigen with an equilibrium dissociation constant (K_{D}) in this paragraph at a pH below 7.5. In embodiments, the humanized antibody is capable of binding a CD73 antigen with an equilibrium dissociation constant (K_{D}) in this paragraph at a pH of less than about 7.5. In embodiments, the humanized antibody is capable of binding a CD73 antigen with an equilibrium dissociation constant (K_{D}) in this paragraph at a pH of less than about 7.0. In embodiments, the humanized antibody is capable of binding a CD73 antigen with an equilibrium dissociation constant (K_{D}) in this paragraph at a pH of less than about 6.5. In embodiments, the humanized antibody is capable of binding a CD73 antigen with an equilibrium dissociation constant (K_{D}) in this paragraph at a pH of less than about 6.0. In embodiments, the humanized antibody is capable of binding a CD73 antigen with an equilibrium dissociation constant (K_{D}) in this paragraph at a pH of less than about 5.5. In embodiments, the humanized antibody is capable of binding a CD73 antigen with an equilibrium dissociation constant (K_{D}) in this paragraph at a pH of less than about 5. In embodiments, the humanized antibody is capable of binding a CD73 antigen with an equilibrium dissociation constant (K_{D}) in this paragraph at a pH of less than about 4.5. In embodiments, the humanized antibody is capable of binding a CD73 antigen with an equilibrium dissociation constant (K_{D}) in this paragraph at a pH from about 6.0 to about 7.0. In embodiments, the humanized antibody is capable of binding a CD73 antigen with an equilibrium dissociation constant (K_{D}) in this paragraph at a pH of about 6.0. In embodiments, the humanized antibody is capable of binding a CD73 antigen with an equilibrium dissociation constant (K_{D}) in this paragraph at a pH of about 6.1. In embodiments, the humanized antibody is capable of binding a CD73 antigen with an equilibrium dissociation constant (K_{D}) in this paragraph at a pH of about 6.2. In embodiments, the humanized antibody is capable of binding a CD73 antigen with an equilibrium dissociation constant (K_{D}) in this paragraph at a pH of about 6.3. In embodiments, the humanized antibody is capable of binding a CD73 antigen with an equilibrium dissociation constant (K_{D}) in this paragraph at a pH of about 6.4. In embodiments, the humanized antibody is capable of binding a CD73 antigen with an equilibrium dissociation constant (K_{D}) in this paragraph at a pH of about 6.5. In embodiments, the humanized antibody is capable of binding a CD73 antigen with an equilibrium dissociation constant (K_{D}) in this paragraph at a pH of about 6.6. In embodiments, the humanized antibody is capable of binding a CD73 antigen with an equilibrium dissociation constant (K_{D}) in this paragraph at a pH of about 6.7. In embodiments, the humanized antibody is capable of binding a CD73 antigen with an equilibrium dissociation constant (K_{D}) in this paragraph at a pH of about 6.8. In embodiments, the humanized antibody is capable of binding a CD73 antigen with an equilibrium dissociation constant (K_{D}) in this paragraph at a pH of about 6.9. In embodiments, the humanized antibody is capable of binding a CD73 antigen with an equilibrium dissociation constant (K_{D}) in this paragraph at a pH of about 7.0.

In embodiments, the humanized antibody is capable of binding a CD73 antigen with an equilibrium dissociation constant (K_{D}) from about 17 to about 25 nM. In embodiments, the humanized antibody is capable of binding a CD73 antigen with an equilibrium dissociation constant (K_{D}) from about 18 to about 25 nM. In embodiments, the humanized antibody is capable of binding a CD73 antigen with an equilibrium dissociation constant (K_{D}) from about 19 to about 25 nM. In embodiments, the humanized antibody is capable of binding a CD73 antigen with an equilibrium dissociation constant (K_{D}) from about 20 to about 25 nM. In embodiments, the humanized antibody is capable of binding a CD73 antigen with an equilibrium dissociation constant (K_{D}) from about 21 to about 25 nM. In embodiments, the humanized antibody is capable of binding a CD73 antigen with an equilibrium dissociation constant (K_{D}) from about 22 to about 25 nM. In embodiments, the humanized antibody is capable of binding a CD73 antigen with an equilibrium dissociation constant (K_{D}) from about 23 to about 25 nM. In embodiments, the humanized antibody is capable of binding a CD73 antigen with an equilibrium dissociation constant (K_{D}) from about 24 to about 25 nM. In embodiments, the humanized antibody is capable of binding a CD73 antigen with an equilibrium dissociation constant (K_{D}) of about 0.5, 1 2, 3, 4, 5, 6, 7, 8, 9, 10, 11, 12, 13, 14, 15, 16, 17, 18, 19, 20, 21, 22, 23, 24, or 25 nM. In embodiments, the humanized antibody is capable of binding a CD73 antigen with an equilibrium dissociation constant (K_{D}) in this paragraph at a pH below 7.5. In embodiments, the humanized antibody is capable of binding a CD73 antigen with an equilibrium dissociation constant (K_{D}) in this paragraph at a pH of less than about 7.5. In embodiments, the humanized antibody is capable of binding a CD73 antigen with an equilibrium dissociation constant (K_{D}) in this paragraph at a pH of less than about 7.0. In embodiments, the humanized antibody is capable of binding a CD73 antigen with an equilibrium dissociation constant (K_{D}) in this paragraph at a pH of less than about 6.5. In embodiments, the humanized antibody is capable of binding a CD73 antigen with an equilibrium dissociation constant (K_{D}) in this paragraph at a pH of less than about 6.0. In embodiments, the humanized antibody is capable of binding a CD73 antigen with an equilibrium dissociation constant (K_{D}) in this paragraph at a pH of less than about 5.5. In embodiments, the humanized antibody is capable of binding a CD73 antigen with an equilibrium dissociation constant (K_{D}) in this paragraph at a pH of less than about 5. In embodiments, the humanized antibody is capable of binding a CD73 antigen with an equilibrium dissociation constant (K_{D}) in this paragraph at a pH of less than about 4.5. In embodiments, the humanized antibody is capable of binding a CD73 antigen with an equilibrium dissociation constant (K_{D}) in this paragraph at a pH from about 6.0 to about 7.0. In embodiments, the humanized antibody is capable of binding a CD73 antigen with an equilibrium dissociation constant (K_{D}) in this paragraph at a pH of about 6.0. In embodiments, the humanized antibody is capable of binding a CD73 antigen with an equilibrium dissociation constant (K_{D}) in this paragraph at a pH of about 6.1. In embodiments, the humanized antibody is capable of binding a CD73 antigen with an equilibrium dissociation constant (K_{D}) in this paragraph at a pH of about 6.2. In embodiments, the humanized antibody is capable of binding a CD73 antigen with an equilibrium dissociation constant (K_{D}) in this paragraph at a pH of about 6.3. In embodiments, the humanized antibody is capable of binding a CD73 antigen with an equilibrium dissociation constant (K_{D}) in this paragraph at a pH of about 6.4. In embodiments, the humanized antibody is capable of binding a CD73 antigen with an equilibrium dissociation constant (K_{D}) in this paragraph at a pH of about 6.5. In embodiments, the humanized antibody is capable of binding a CD73 antigen with an equilibrium dissociation constant (K_{D}) in this paragraph at a pH of about 6.6. In embodiments, the humanized antibody is capable of binding a CD73 antigen with an equilibrium dissociation constant (K_{D}) in this paragraph at a pH of about 6.7. In embodiments, the humanized antibody is capable of binding a CD73 antigen with an equilibrium dissociation constant (K_{D}) in this paragraph at a pH of about 6.8. In embodiments, the humanized antibody is capable of binding a CD73 antigen with an equilibrium dissociation constant (K_{D}) in this paragraph at a pH of about 6.9. In embodiments, the humanized antibody is capable of binding a CD73 antigen with an equilibrium dissociation constant (K_{D}) in this paragraph at a pH of about 7.0.

In embodiments, the humanized antibody is capable of binding a CD73 antigen with an equilibrium dissociation constant (K_{D}) of about 7.1 nM. In embodiments, the humanized antibody is capable of binding a CD73 antigen with an equilibrium dissociation constant (K_{D}) of about 6.9 nM. In embodiments, the humanized antibody is capable of binding a CD73 antigen with an equilibrium dissociation constant (K_{D}) of about 9.4 nM. In embodiments, the humanized antibody is capable of binding a CD73 antigen with an equilibrium dissociation constant (K_{D}) of about 19.5 nM. In embodiments, the humanized antibody is capable of binding a CD73 antigen with an equilibrium dissociation constant (K_{D}) of about 17.8 nM. In embodiments, the humanized antibody is capable of binding a CD73 antigen with an equilibrium dissociation constant (K_{D}) of about 15.9 nM. In embodiments, the humanized antibody is capable of binding a CD73 antigen with an equilibrium dissociation constant (K_{D}) in this paragraph at a pH below 7.5. In embodiments, the humanized antibody is capable of binding a CD73 antigen with an equilibrium dissociation constant (K_{D}) in this paragraph at a pH of less than about 7.5. In embodiments, the humanized antibody is capable of binding a CD73 antigen with an equilibrium dissociation constant (K_{D}) in this paragraph at a pH of less than about 7.0. In embodiments, the humanized antibody is capable of binding a CD73 antigen with an equilibrium dissociation constant (K_{D}) in this paragraph at a pH of less than about 6.5. In embodiments, the humanized antibody is capable of binding a CD73 antigen with an equilibrium dissociation constant (K_{D}) in this paragraph at a pH of less than about 6.0. In embodiments, the humanized antibody is capable of binding a CD73 antigen with an equilibrium dissociation constant (K_{D}) in this paragraph at a pH of less than about 5.5. In embodiments, the humanized antibody is capable of binding a CD73 antigen with an equilibrium dissociation constant (K_{D}) in this paragraph at a pH of less than about 5. In embodiments, the humanized antibody is capable of binding a CD73 antigen with an equilibrium dissociation constant (K_{D}) in this paragraph at a pH of less than about 4.5. In embodiments, the humanized antibody is capable of binding a CD73 antigen with an equilibrium dissociation constant (K_{D}) in this paragraph at a pH from about 6.0 to about 7.0. In embodiments, the humanized antibody is capable of binding a CD73 antigen with an equilibrium dissociation constant (K_{D}) in this paragraph at a pH of about 6.0. In embodiments, the humanized antibody is capable of binding a CD73 antigen with an equilibrium dissociation constant (K_{D}) in this paragraph at a pH of about 6.1. In embodiments, the humanized antibody is capable of binding a CD73 antigen with an equilibrium dissociation constant (K_{D}) in this paragraph at a pH of about 6.2. In embodiments, the humanized antibody is capable of binding a CD73 antigen with an equilibrium dissociation constant (K_{D}) in this paragraph at a pH of about 6.3. In embodiments, the humanized antibody is capable of binding a CD73 antigen with an equilibrium dissociation constant (K_{D}) in this paragraph at a pH of about 6.4. In embodiments, the humanized antibody is capable of binding a CD73 antigen with an equilibrium dissociation constant (K_{D}) in this paragraph at a pH of about 6.5. In embodiments, the humanized antibody is capable of binding a CD73 antigen with an equilibrium dissociation constant (K_{D}) in this paragraph at a pH of about 6.6. In embodiments, the humanized antibody is capable of binding a CD73 antigen with an equilibrium dissociation constant (K_{D}) in this paragraph at a pH of about 6.7. In embodiments, the humanized antibody is capable of binding a CD73 antigen with an equilibrium dissociation constant (K_{D}) in this paragraph at a pH of about 6.8. In embodiments, the humanized antibody is capable of binding a CD73 antigen with an equilibrium dissociation constant (K_{D}) in this paragraph at a pH of about 6.9. In embodiments, the humanized antibody is capable of binding a CD73 antigen with an equilibrium dissociation constant (K_{D}) in this paragraph at a pH of about 7.0.

In embodiments, the antibody is capable of binding a CD73 antigen with an equilibrium dissociation constant (K_{D}) of about 0.64 nM.

In embodiments, the antibody is capable of binding a CD73 antigen at a pH of less than about 7.5. In embodiments, the antibody, is capable of binding a CD73 antigen at a pH of less than about 7.0. In embodiments, the antibody, is capable of binding a CD73 antigen at a pH of less than about 6.5. In embodiments, the antibody, is capable of binding a CD73 antigen at a pH of less than about 6.0. In embodiments, the antibody, is capable of binding a CD73 antigen at a pH of less than about 5.5. In embodiments, the antibody, is capable of binding a CD73 antigen at a pH of less than about 5. In embodiments, the antibody, is capable of binding a CD73 antigen at a pH of less than about 4.5. In embodiments, the antibody is capable of binding a CD73 antigen at a pH from about 6.0 to about 7.0. In embodiments, the antibody is capable of binding a CD73 antigen at a pH of about 6.0. In embodiments, the antibody is capable of binding a CD73 antigen at a pH of about 6.1. In embodiments, the antibody is capable of binding a CD73 antigen at a pH of about 6.2. In embodiments, the antibody is capable of binding a CD73 antigen at a pH of about 6.3. In embodiments, the antibody is capable of binding a CD73 antigen at a pH of about 6.4. In embodiments, the antibody is capable of binding a CD73 antigen at a pH of about 6.5. In embodiments, the antibody is capable of binding a CD73 antigen at a pH of about 6.6. In embodiments, the antibody is capable of binding a CD73 antigen at a pH of about 6.7. In embodiments, the antibody is capable of binding a CD73 antigen at a pH of about 6.8. In embodiments, the antibody is capable of binding a CD73 antigen at a pH of about 6.9. In embodiments, the antibody is capable of binding a CD73 antigen at a pH of about 7.0.

In embodiments, the antibody is capable of binding a CD73 antigen at a pH of about 6.3. In embodiments, the antibody is capable of binding a CD73 antigen at a pH of 6.3.

In embodiments, the antibody further includes a glutamine at a position corresponding to Kabat position 297.

In embodiments, the antibody is bound to a CD73 antigen. In embodiments, the CD73 antigen forms part of a cell. In embodiments, the cell is a tumor cell. In embodiments, the cell is a cancer cell. In embodiments, the cell is a non-cancer cell. In embodiments, the cell is an immune cell. In embodiments, the cell is a stromal cell (e.g., non-tumor cells including, for example, fibroblasts, pericytes, endothelial cells, etc.). In embodiments, the cell is a lymphoid cell. In embodiments, the cell is a T cell. In embodiments, the cell is a cancer cell. In embodiments, the CD73 antigen forms part of a tumor cell and not a stromal cell. In embodiments, the CD73 antigen forms part of a stromal cell and not a tumor cell. In embodiments, the CD73 antigen forms part of a stromal cell and a tumor cell.

In an aspect, a method of treating cancer in a subject in need thereof is provided. The method includes administering to the subject a therapeutically effective amount of a humanized IgG1 antibody, wherein the subject expresses an elevated level of CD73 relative to a standard control and wherein the humanized IgG1 antibody includes a humanized light chain variable region and a humanized heavy chain variable region, wherein the humanized light chain variable region includes a mouse CDR L1 as set forth in SEQ ID NO:1, a mouse CDR L2 as set forth in SEQ ID NO:2, a mouse CDR L3 as set forth in SEQ ID NO:3; and wherein the humanized heavy chain variable region includes a mouse CDR H1 as set forth in SEQ ID NO:4, a mouse CDR H2 as set forth in SEQ ID NO:5, and a mouse CDR H3 as set forth in SEQ ID NO:6.

In an aspect, a method of treating cancer in a subject in need thereof is provided. The method includes administering to the subject a therapeutically effective amount of a humanized IgG4 antibody, wherein the subject expresses an elevated level of CD73 relative to a standard control and wherein the humanized IgG4 antibody includes a humanized light chain variable region and a humanized heavy chain variable region, wherein the humanized light chain variable region includes a mouse CDR L1 as set forth in SEQ ID NO:1, a mouse CDR L2 as set forth in SEQ ID NO:2, a mouse CDR L3 as set forth in SEQ ID NO:3; and wherein the humanized heavy chain variable region includes a mouse CDR H1 as set forth in SEQ ID NO:4, a mouse CDR H2 as set forth in SEQ ID NO:5, and a mouse CDR H3 as set forth in SEQ ID NO:6.

The humanized light chain variable region may include a mouse CDR L1 as set forth in SEQ ID NO:1, a mouse CDR L2 as set forth in SEQ ID NO:2, or a mouse CDR L3 as set forth in SEQ ID NO:3. The humanized light chain variable region may include a mouse CDR L1 as set forth in SEQ ID NO:1, a mouse CDR L2 as set forth in SEQ ID NO:2, and a mouse CDR L3 as set forth in SEQ ID NO:3. The humanized heavy chain variable region may include a mouse CDR H1 as set forth in SEQ ID NO:4, a mouse CDR H2 as set forth in SEQ ID NO:5, or a mouse CDR H3 as set forth in SEQ ID NO:6. The humanized heavy chain variable region may include a mouse CDR H1 as set forth in SEQ ID NO:4, a mouse CDR H2 as set forth in SEQ ID NO:5, and a mouse CDR H3 as set forth in SEQ ID NO:6. In embodiments, the humanized light chain variable region includes a mouse CDR L1 as set forth in SEQ ID NO:1. In embodiments, the humanized light chain variable region includes a mouse CDR L2 as set forth in SEQ ID NO:2. In embodiments, the humanized light chain variable region includes a mouse CDR L3 as set forth in SEQ ID NO:3. In embodiments, the humanized heavy chain variable region includes a mouse CDR H1 as set forth in SEQ ID NO:4. In embodiments, the humanized heavy chain variable region includes a mouse CDR H2 as set forth in SEQ ID NO:5. In embodiments, the humanized light chain variable region includes a mouse CDR H3 as set forth in SEQ ID NO:6.

In embodiments, the humanized light chain variable region further includes a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85 or a phenylalanine at a position corresponding to Kabat position 87.

In embodiments, the humanized light chain variable region further includes a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, an isoleucine at a position corresponding to Kabat position 78, a tyrosine at a position corresponding to Kabat position 85 and a phenylalanine at a position corresponding to Kabat position 87.

In embodiments, the humanized heavy chain variable region further includes an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 or a glutamic acid at a position corresponding to Kabat position 81.

In embodiments, the humanized heavy chain variable region further includes an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 and a glutamic acid at a position corresponding to Kabat position 81.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

In embodiments, the humanized heavy chain variable region includes the sequence of SEQ ID NO:7. In embodiments, the humanized heavy chain variable region is the sequence of SEQ ID NO:7. In embodiments, the humanized light chain variable region includes the sequence of SEQ ID NO:37. In embodiments, the humanized light chain variable region is the sequence of SEQ ID NO:37. In embodiments, the antibody further includes a glutamine at a position corresponding to Kabat position 297.

In embodiments, the antibody is bound to a CD73 antigen. In embodiments, the CD73 antigen forms part of a cell. In embodiments, the cell is a tumor cell. In embodiments, the cell is a cancer cell. In embodiments, the cell is a non-cancer cell. In embodiments, the cell is an immune cell. In embodiments, the cell is a stromal cell (e.g., non-tumor cells including, for example, fibroblasts, pericytes, endothelial cells, etc.). In embodiments, the cell is a T cell. In embodiments, the cell is a cancer cell. In embodiments, the CD73 antigen forms part of a tumor cell and not a stromal cell. In embodiments, the CD73 antigen forms part of a stromal cell and not a tumor cell. In embodiments, the CD73 antigen forms part of a stromal cell and a tumor cell.

In an aspect, a method of treating cancer in a subject in need thereof is provided. The method includes administering to the subject a therapeutically effective amount of an anti-CD73 antibody, wherein the subject expresses an elevated level of CD73 relative to a standard control and wherein the anti-CD73 antibody binds the same epitope as a 1E9 antibody.

In embodiments, the 1E9 antibody includes a humanized light chain variable region including a mouse CDR L1, mouse CDR L2, or mouse CDR L3 and a humanized heavy chain variable region including a mouse CDR H1, mouse CDR H2, or mouse CDR H3.

In embodiments, the humanized light chain variable region further includes a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85 or a phenylalanine at a position corresponding to Kabat position 87.

In embodiments, the humanized light chain variable region further includes a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85 and a phenylalanine at a position corresponding to Kabat position 87.

In embodiments, the humanized heavy chain variable region further includes an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 or a glutamic acid at a position corresponding to Kabat position 81.

In embodiments, the humanized heavy chain variable region further includes an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 and a glutamic acid at a position corresponding to Kabat position 81.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104, and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104, and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104, and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104, and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

In embodiments, the humanized heavy chain variable region includes the sequence of SEQ ID NO:7. In embodiments, the humanized heavy chain variable region is the sequence of SEQ ID NO:7. In embodiments, the humanized light chain variable region includes the sequence of SEQ ID NO:37. In embodiments, the humanized light chain variable region is the sequence of SEQ ID NO:37. In embodiments, antibody further includes a glutamine at a position corresponding to Kabat position 297.

In embodiments, the antibody is bound to a CD73 antigen. In embodiments, the CD73 antigen forms part of a cell. In embodiments, the cell is a tumor cell. In embodiments, the cell is a cancer cell. In embodiments, the cell is a non-cancer cell. In embodiments, the cell is an immune cell. In embodiments, the cell is a stromal cell (e.g., non-tumor cells including, for example, fibroblasts, pericytes, endothelial cells, etc.). In embodiments, the cell is a T cell. In embodiments, the cell is a cancer cell. In embodiments, the CD73 antigen forms part of a tumor cell and not a stromal cell. In embodiments, the CD73 antigen forms part of a stromal cell and not a tumor cell. In embodiments, the CD73 antigen forms part of a stromal cell and a tumor cell.

The methods provided herein including embodiments thereof of may include a further step of detecting an elevated level of CD73 prior to administering a therapeutically effective amount of an antibody provided herein (e.g., anti-CD73 antibody, 1E9 antibody, humanized 1E9 antibody, IgG1 antibody, IgG4 antibody). Thus, in embodiments, the methods of treating provided herein including embodiments thereof, include, prior to the administering, detecting an elevated level of CD73 in the subject relative to a standard control.

### Combination Treatment Methods

The methods of treating provided herein including embodiments thereof, may include administration of a second therapeutic agent. Therefore, the methods of treatment as provided herein include administering an antibody as provided herein (e.g., anti-CD73 antibody, 1E9 antibody, humanized 1E9 antibody, IgG1 antibody, IgG4 antibody) in combination with a second therapeutic agent. The second therapeutic agent may be any composition useful in treating or preventing cancer.

In embodiments, the method includes administering a therapeutically effective amount of a second therapeutic agent.

The second therapeutic agent useful for the methods provided herein may be a compound, drug, antagonist, inhibitor, or modulator, having antineoplastic properties or the ability to inhibit the growth or proliferation of cells. In embodiments, the second therapeutic agent is a chemotherapeutic. “Chemotherapeutic” or “chemotherapeutic agent” is used in accordance with its plain ordinary meaning and refers to a chemical composition or compound having antineoplastic properties or the ability to inhibit the growth or proliferation of cells. In embodiments, the second therapeutic agent is radiation therapy. In embodiments, the second therapeutic agent is an agent approved by the FDA or similar regulatory agency of a country other than the USA, for treating cancer.

In embodiments, the second therapeutic agent is a compound. In embodiments, the compound is a A2A receptor antagonist.

In embodiments, the A2A receptor antagonist is a compound of formula:

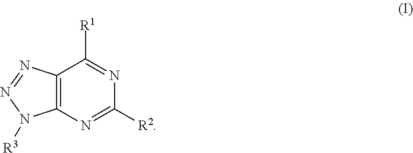

In formula (I), R¹ is independently hydrogen, halogen, —CX^{a}₃, —CN, —SO₂Cl, —SOₙ₁R⁹, —SOᵥ₁NR⁹R¹⁰, —NHNH₂, —ONR⁹R¹⁰, —NHC═(O)NHNH₂, —NHC═(O)NR⁹R¹⁰, —N(O)ₘ₁, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is independently hydrogen, halogen, —CX^{b}₃, —CN, —SO₂Cl, —SOᵥ₂R¹¹, —SOᵥ₂NR¹¹R¹², —NHNH₂, —ONR¹¹R¹², —NHC═(O)NHNH₂, —NHC═(O)NR¹¹R¹², —N(O)ₘ₂, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is independently hydrogen, halogen, —CX^{c}₃, —CN, —SO₂Cl, —SOₙ₃R¹³, —SOᵥ₃NR¹³R¹⁴, —NHNH₂, —ONR¹³R¹⁴, —NHC═(O)NHNH₂, —NHC═(O)NR¹³R¹⁴, —N(O)ₘ₃, —NR¹³R¹⁴, —NH—O—R¹³, —C(O)R¹³, —C(O)—OR¹³, —C(O)NR¹³R¹⁴, —OR¹³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁹, R¹⁰, R¹¹, R¹², R¹³ and R¹⁴ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁹, R¹⁰, R¹¹, R¹², R¹³ and R¹⁴ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X^{a}, X^{b} and X^{c} are independently —F, —Cl, —Br, or —I.

The symbols n₁, n₂ and n₃ are independently an integer from 0 to 4. In embodiments, n₁ is 0. In embodiments, n₁ is 1. In embodiments, n₁ is 3. In embodiments, n₁ is 4. In embodiments, n₂ is 0. In embodiments, n₂ is 1. In embodiments, n₂ is 3. In embodiments, n₂ is 4. In embodiments, n₃ is 0. In embodiments, n₃ is 1. In embodiments, n₃ is 3. In embodiments, n₃ is 4.

The symbols m₁, m₂ and m₃ are independently an integer from 1 to 2. In embodiments, m₁ is 0. In embodiments, m₁ is 1. In embodiments, m₁ is 2. In embodiments, m₂ is 0. In embodiments, m₂ is 1. In embodiments, m₂ is 2. In embodiments, m₃ is 0. In embodiments, m₃ is 1. In embodiments, m₂ is 2.

The symbols v₁, v₂ and v₃ are independently an integer from 1 to 2. In embodiments, v₁ is 0. In embodiments, v₁ is 1. In embodiments, v₁ is 2. In embodiments, v₂ is 0. In embodiments, v₂ is 1. In embodiments, v₂ is 2. In embodiments, v₃ is 0. In embodiments, v₃ is 1. In embodiments, v₃ is 2.

In embodiments, R¹ is independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl. R¹ may be R^{1A}-substituted or unsubstituted (e.g., C₁-C₂₀ or C₁-C₆) alkyl, R^{1A}-substituted or unsubstituted (e.g., 2 to 20 membered or 2 to 6 membered) heteroalkyl, R^{1A}-substituted or unsubstituted (e.g., C₃-C₈ or C₅-C₇) cycloalkyl, R^{1A}-substituted or unsubstituted (e.g., 3 to 8 membered or 3 to 6 membered) heterocycloalkyl, R^{1A}-substituted or unsubstituted (e.g., C₅-C₁₀ or C₅-C₆) aryl, or R^{1A}-substituted or unsubstituted (e.g., 5 to 10 membered or 5 to 6 membered) heteroaryl.

In embodiments, R^{1A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl. R^{1A} may be R^{1B}-substituted or unsubstituted (e.g., C₁-C₂₀ or C₁-C₆) alkyl, R^{1B}-substituted or unsubstituted (e.g., 2 to 20 membered or 2 to 6 membered) heteroalkyl, R^{1B}-substituted or unsubstituted (e.g., C₃-C₈ or C₅-C₇) cycloalkyl, R^{1B}-substituted or unsubstituted (e.g., 3 to 8 membered or 3 to 6 membered) heterocycloalkyl, R^{1B}-substituted or unsubstituted (e.g., C₅-C₁₀ or C₅-C₆) aryl, or R^{1B}-substituted or unsubstituted (e.g., 5 to 10 membered or 5 to 6 membered) heteroaryl.

In embodiments, R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl. R^{1B} may be R^{1C}-substituted or unsubstituted (e.g., C₁-C₂₀ or C₁-C₆) alkyl, R^{1C}-substituted or unsubstituted (e.g., 2 to 20 membered or 2 to 6 membered) heteroalkyl, R^{1C}-substituted or unsubstituted (e.g., C₃-C₈ or C₅-C₇) cycloalkyl, R^{1C}-substituted or unsubstituted (e.g., 3 to 8 membered or 3 to 6 membered) heterocycloalkyl, R^{1C}-substituted or unsubstituted (e.g., C₅-C₁₀ or C₅-C₆) aryl, or R^{1C}-substituted or unsubstituted (e.g., 5 to 10 membered or 5 to 6 membered) heteroaryl.

R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. R^{1C} may be independently unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, R¹ is independently R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or s R^{1A}-substituted or unsubstituted heteroaryl. In embodiments, R¹ is R^{1A}-substituted or unsubstituted (e.g., 5 to 10 membered or 5 to 6 membered) heteroaryl. In embodiments, R¹ is unsubstituted 5 to 6 membered heteroaryl. In embodiments, R¹ is R^{1A}-substituted 5 to 6 membered heteroaryl. In embodiments, R¹ is unsubstituted 5 membered heteroaryl. In embodiments, R¹ is R^{1A}-substituted 5 membered heteroaryl. In embodiments, R¹ is R^{1A}-substituted furanyl.

In embodiments, R^{1A} is R^{1B}-substituted or unsubstituted (e.g., C₁-C₂₀ or C₁-C₆) alkyl. In embodiments, R^{1A} is R^{1B}-substituted C₁-C₆ alkyl. In embodiments, R^{1A} is unsubstituted C₁-C₆ alkyl. In embodiments, R^{1A} is R^{1B}-substituted C₁-C₄ alkyl. In embodiments, R^{1A} is unsubstituted C₁-C₄ alkyl. In embodiments, R^{1A} is R^{1B}-substituted C₁-C₃ alkyl. In embodiments, R^{1A} is unsubstituted C₁-C₃ alkyl. In embodiments, R^{1A} is methyl.

In embodiments, R² is independently hydrogen, halogen, —CX^{b}₃, —CN, —SO₂Cl, —SOₙ₂R¹¹, —SOᵥ₂NR¹¹R¹², —NHNH₂, —ONR¹¹R¹², —NHC═(O)NHNH₂, —NHC═(O)NR¹¹R¹², —N(O)ₘ₂, —NR¹¹R¹², —NH—O—R¹¹, —C(O)R¹¹, —C(O)—OR¹¹, —C(O)NR¹¹R¹², or —OR¹¹. In embodiments of the methods provided herein, R² is independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, R² is —NR¹¹R¹². In embodiments, R¹¹ and R¹² are independently hydrogen or substituted or unsubstituted (e.g., C₁-C₂₀ or C₁-C₆) alkyl. In embodiments, R¹¹ and R¹² are independently substituted or unsubstituted C₁-C₆ alkyl. In embodiments, R¹¹ and R¹² are independently substituted or unsubstituted C₁-C₄ alkyl. In embodiments, R¹¹ and R¹² are independently substituted or unsubstituted C₁-C₃ alkyl. In embodiments, R¹¹ and R¹² are independently unsubstituted C₁-C₆ alkyl. In embodiments, R¹¹ and R¹² are independently substituted or unsubstituted C₁-C₄ alkyl. In embodiments, R¹¹ and R¹² are independently unsubstituted C₁-C₃ alkyl. In embodiments, R¹¹ and R¹² are independently hydrogen.

In embodiments, R³ is independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴-substituted or unsubstituted alkyl, R⁴-substituted or unsubstituted heteroalkyl, R⁴-substituted or unsubstituted cycloalkyl, R⁴-substituted or unsubstituted heterocycloalkyl, R⁴-substituted or unsubstituted aryl, or R⁴-substituted or unsubstituted heteroaryl. R³ may be R⁴-substituted or unsubstituted (e.g., C₁-C₂₀ or C₁-C₆) alkyl, R⁴-substituted or unsubstituted (e.g., 2 to 20 membered or 2 to 6 membered) heteroalkyl, R⁴-substituted or unsubstituted (e.g., C₃-C₈ or C₅-C₇) cycloalkyl, R⁴-substituted or unsubstituted (e.g., 3 to 8 membered or 3 to 6 membered) heterocycloalkyl, R⁴-substituted or unsubstituted (e.g., C₅-C₁₀ or C₅-C₆) aryl, or R⁴-substituted or unsubstituted (e.g., 5 to 10 membered or 5 to 6 membered) heteroaryl.

R⁴ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵-substituted or unsubstituted alkyl, R⁵-substituted or unsubstituted heteroalkyl, R⁵-substituted or unsubstituted cycloalkyl, R⁵-substituted or unsubstituted heterocycloalkyl, R⁵-substituted or unsubstituted aryl, or R⁵-substituted or unsubstituted heteroaryl. R⁴ may be R⁵-substituted or unsubstituted (e.g., C₁-C₂₀ or C₁-C₆) alkyl, R⁵-substituted or unsubstituted (e.g., 2 to 20 membered or 2 to 6 membered) heteroalkyl, R⁵-substituted or unsubstituted (e.g., C₃-C₈ or C₅-C₇) cycloalkyl, R⁵-substituted or unsubstituted (e.g., 3 to 8 membered or 3 to 6 membered) heterocycloalkyl, R⁵-substituted or unsubstituted (e.g., C₅-C₁₀ or C₅-C₆) aryl, or R⁵-substituted or unsubstituted (e.g., 5 to 10 membered or 5 to 6 membered) heteroaryl.

R⁵ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁶-substituted or unsubstituted alkyl, R⁶-substituted or unsubstituted heteroalkyl, R⁶-substituted or unsubstituted cycloalkyl, R⁶-substituted or unsubstituted heterocycloalkyl, R⁶-substituted or unsubstituted aryl, or R⁶-substituted or unsubstituted heteroaryl. R⁵ may be R⁶-substituted or unsubstituted (e.g., C₁-C₂₀ or C₁-C₆) alkyl, R⁶-substituted or unsubstituted (e.g., 2 to 20 membered or 2 to 6 membered) heteroalkyl, R⁶-substituted or unsubstituted (e.g., C₃-C₈ or C₅-C₇) cycloalkyl, R⁶-substituted or unsubstituted (e.g., 3 to 8 membered or 3 to 6 membered) heterocycloalkyl, R⁶-substituted or unsubstituted (e.g., C₅-C₁₀ or C₅-C₆) aryl, or R⁶-substituted or unsubstituted (e.g., 5 to 10 membered or 5 to 6 membered) heteroaryl.

R⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁷-substituted or unsubstituted alkyl, R⁷-substituted or unsubstituted heteroalkyl, R⁷-substituted or unsubstituted cycloalkyl, R⁷-substituted or unsubstituted heterocycloalkyl, R⁷-substituted or unsubstituted aryl, or R⁷-substituted or unsubstituted heteroaryl. R⁶ may be R⁷-substituted or unsubstituted (e.g., C₁-C₂₀ or C₁-C₆) alkyl, R⁷-substituted or unsubstituted (e.g., 2 to 20 membered or 2 to 6 membered) heteroalkyl, R⁷-substituted or unsubstituted (e.g., C₃-C₈ or C₅-C₇) cycloalkyl, R⁷-substituted or unsubstituted (e.g., 3 to 8 membered or 3 to 6 membered) heterocycloalkyl, R⁷-substituted or unsubstituted (e.g., C₅-C₁₀ or C₅-C₆) aryl, or R⁷-substituted or unsubstituted (e.g., 5 to 10 membered or 5 to 6 membered) heteroaryl.

In embodiments, R³ is independently hydrogen, halogen, R⁴-substituted or unsubstituted alkyl, R⁴-substituted or unsubstituted heteroalkyl, R⁴-substituted or unsubstituted cycloalkyl, R⁴-substituted or unsubstituted heterocycloalkyl, R⁴-substituted or unsubstituted aryl, or R⁴-substituted or unsubstituted heteroaryl. In embodiments, R³ is independently R⁴-substituted or unsubstituted (e.g., C₁-C₂₀ or C₁-C₆) alkyl. In embodiments, R³ is independently R⁴-substituted or unsubstituted C₁-C₆ alkyl. In embodiments, R³ is independently R⁴-substituted or unsubstituted C₁-C₅ alkyl. In embodiments, R³ is independently R⁴-substituted or unsubstituted C₁-C₄ alkyl. In embodiments, R³ is independently R⁴-substituted or unsubstituted C₁-C₃ alkyl. In embodiments, R³ is independently unsubstituted C₁-C₆ alkyl. In embodiments, R³ is independently unsubstituted C₁-C₅ alkyl. In embodiments, R³ is independently R⁴-unsubstituted C₁-C₄ alkyl. In embodiments, R³ is independently unsubstituted C₁-C₃ alkyl. In embodiments, R³ is independently R⁴-substituted C₁-C₆ alkyl. In embodiments, R³ is independently R⁴-substituted C₁-C₅ alkyl. In embodiments, R³ is independently R⁴-substituted C₁-C₄ alkyl. In embodiments, R³ is independently R⁴-substituted C₁-C₃ alkyl. In embodiments, R³ is R⁴-substituted C₁ alkyl.

In embodiments, R⁴ is R⁵-substituted or unsubstituted (e.g., C₁-C₂₀ or C₁-C₆) alkyl, R⁵-substituted or unsubstituted (e.g., 2 to 20 membered or 2 to 6 membered) heteroalkyl, R⁵-substituted or unsubstituted (e.g., C₃-C₈ or C₅-C₇) cycloalkyl, R⁵-substituted or unsubstituted (e.g., 3 to 8 membered or 3 to 6 membered) heterocycloalkyl, R⁵-substituted or unsubstituted (e.g., C₅-C₁₀ or C₅-C₆) aryl, or R⁵-substituted or unsubstituted (e.g., 5 to 10 membered or 5 to 6 membered) heteroaryl. In embodiments, R⁴ is R⁵-substituted or unsubstituted 5 to 6 membered heteroaryl. In embodiments, R⁴ is R⁵-substituted or unsubstituted 6 membered heteroaryl. In embodiments, R⁴ is unsubstituted 6 membered heteroaryl. In embodiments, R⁴ is R⁵-substituted 6 membered heteroaryl. In embodiments, R⁴ is R⁵-substituted pyridinyl.

In embodiments, R⁵ is R⁶-substituted or unsubstituted (e.g., C₁-C₂₀ or C₁-C₆) alkyl, R⁶-substituted or unsubstituted (e.g., 2 to 20 membered or 2 to 6 membered) heteroalkyl, R⁶-substituted or unsubstituted (e.g., C₃-C₈ or C₅-C₇) cycloalkyl, R⁶-substituted or unsubstituted (e.g., 3 to 8 membered or 3 to 6 membered) heterocycloalkyl, R⁶-substituted or unsubstituted (e.g., C₅-C₁₀ or C₅-C₆) aryl, or R⁶-substituted or unsubstituted (e.g., 5 to 10 membered or 5 to 6 membered) heteroaryl. In embodiments, R⁵ is R⁶-substituted or unsubstituted 2 to 6 membered heteroalkyl. In embodiments, R⁵ is R⁶-substituted or unsubstituted 2 to 5 membered heteroalkyl. In embodiments, R⁵ is R⁶-substituted or unsubstituted 2 to 4 membered heteroalkyl. In embodiments, R⁵ is R⁶-substituted or unsubstituted 2 to 3 membered heteroalkyl. In embodiments, R⁵ is R⁶-substituted or unsubstituted 2 membered heteroalkyl. In embodiments, R⁵ is unsubstituted 2 to 6 membered heteroalkyl. In embodiments, R⁵ is unsubstituted 2 to 5 membered heteroalkyl. In embodiments, R⁵ is unsubstituted 2 to 4 membered heteroalkyl. In embodiments, R⁵ unsubstituted 2 to 3 membered heteroalkyl. In embodiments, R⁵ is unsubstituted 2 membered heteroalkyl. In embodiments, R⁵ is R⁶-substituted 2 to 6 membered heteroalkyl. In embodiments, R⁵ is R⁶-substituted 2 to 5 membered heteroalkyl. In embodiments, R⁵ is R⁶-substituted 2 to 4 membered heteroalkyl. In embodiments, R⁵ is R⁶-substituted 2 to 3 membered heteroalkyl. In embodiments, R⁵ is R⁶-substituted 2 membered heteroalkyl.

In embodiments, R⁶ is R⁷-substituted or unsubstituted (e.g., C₁-C₂₀ or C₁-C₆) alkyl, R⁷-substituted or unsubstituted (e.g., 2 to 20 membered or 2 to 6 membered) heteroalkyl, R⁷-substituted or unsubstituted (e.g., C₃-C₈ or C₅-C₇) cycloalkyl, R⁷-substituted or unsubstituted (e.g., 3 to 8 membered or 3 to 6 membered) heterocycloalkyl, R⁷-substituted or unsubstituted (e.g., C₅-C₁₀ or C₅-C₆) aryl, or R⁷-substituted or unsubstituted (e.g., 5 to 10 membered or 5 to 6 membered) heteroaryl. In embodiments, R⁶ is R⁷-substituted or unsubstituted 3 to 6 membered heterocycloalkyl. In embodiments, R⁶ is R⁷-substituted or unsubstituted 5 membered heterocycloalkyl. In embodiments, R⁶ is R⁷-substituted 5 membered heterocycloalkyl. In embodiments, R⁶ is unsubstituted 5 membered heterocycloalkyl. In embodiments, R⁶ is unsubstituted tetrahydrofuranyl.

In embodiments of the methods provided herein, R⁹, R¹⁰, R¹¹, R¹², R¹³ and R¹⁴ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

In embodiments, R¹ is R^{1A}-substituted furanyl. In one further embodiment, R^{1A} is methyl. In another further embodiment, R² is —NR¹¹R¹². In another further embodiment, R¹¹ and R¹² are independently hydrogen. In yet another further embodiment, R³ is R⁴-substituted C₁ alkyl. In another further embodiment, R⁴ is R⁵-substituted pyridinyl. In yet another further embodiment, R⁵ is R⁶-substituted 2 membered heteroalkyl. In another further embodiments, R⁶ is unsubstituted tetrahydrofuranyl.

In embodiments, the A2A receptor antagonist is a compound of formula:

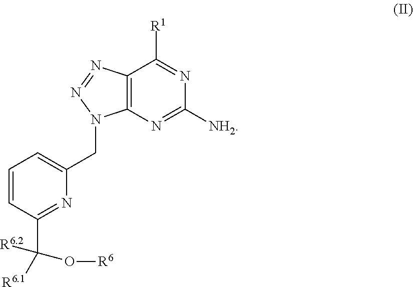

In formula (II), R⁶, R^{6.1} and R^{6.2} are independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁶, R^{6.1} and R^{6.2} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{6.1} and R^{6.2} are hydrogen and R6 is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{6.1} and R^{6.2} are hydrogen and R6 is substituted or unsubstituted heterocycloalkyl. In embodiments, R^{6.1} and R^{6.2} are hydrogen and R6 is unsubstituted heterocycloalkyl. In embodiments, R¹ is substituted (e.g. with an unsubstituted C₁-C₅ alkyl) or unsubstituted heteroaryl. In embodiments, R¹ is substituted (e.g. with an unsubstituted C₁-C₅ alkyl) or unsubstituted furanyl. In embodiments, R¹ is methyl-substituted furanyl.

In formula (II), R¹ and R⁶ are as described above (e.g., R⁶ may be R⁷-substituted or unsubstituted 3 to 6 membered heterocycloalkyl and R¹ may be R^{1A}-substituted 5 to 6 membered heteroaryl). Thus, in embodiments, R⁶ is unsubstituted tetrahydrofuranyl and R¹ is R^{1A}-substituted furanyl.

In formula (II), R^{6.1} may be independently hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{7.1}-substituted or unsubstituted alkyl, R^{7.1}-substituted or unsubstituted heteroalkyl, R^{7.1}-substituted or unsubstituted cycloalkyl, R^{7.1}-substituted or unsubstituted heterocycloalkyl, R^{7.1}-substituted or unsubstituted aryl, or R^{7.1}-substituted or unsubstituted heteroaryl. R^{6.1} may be R^{7.1}-substituted or unsubstituted (e.g., C₁-C₂₀ or C₁-C₆) alkyl, R^{7.1}-substituted or unsubstituted (e.g., 2 to 20 membered or 2 to 6 membered) heteroalkyl, R^{7.1}-substituted or unsubstituted (e.g., C₃-C₈ or C₅-C₇) cycloalkyl, R^{7.1}-substituted or unsubstituted (e.g., 3 to 8 membered or 3 to 6 membered) heterocycloalkyl, R^{7.1}-substituted or unsubstituted (e.g., C₅-C₁₀ or C₅-C₆) aryl, or R^{7.1}-substituted or unsubstituted (e.g., 5 to 10 membered or 5 to 6 membered) heteroaryl. In embodiments, R⁶ is R^{7.1}-substituted or unsubstituted C₁-C₆ alkyl. In embodiments, R⁶ is R^{7.1}-substituted or unsubstituted C₁-C₅ alkyl. In embodiments, R^{6.1} is R^{7.1}-substituted or unsubstituted C₁-C₄ alkyl. In embodiments, R^{6.1} is R^{7.1}-substituted or unsubstituted C₁-C₃ alkyl. In embodiments, R^{6.1} is R^{7.1}-substituted C₁-C₆ alkyl. In embodiments, R^{6.1} is R^{7.1}-substituted C₁-C₅ alkyl. In embodiments, R^{6.1} is R^{7.1}-substituted C₁-C₄ alkyl. In embodiments, R^{6.1} is R^{7.1}-substituted C₁-C₃ alkyl. In embodiments, R^{6.1} is unsubstituted C₁-C₆ alkyl. In embodiments, R^{6.1} is unsubstituted C₁-C₅ alkyl. In embodiments, R^{6.1} is unsubstituted C₁-C₄ alkyl. In embodiments, R^{6.1} is unsubstituted C₁-C₃ alkyl. In embodiments, R^{6.1} is unsubstituted methyl.

R^{6.2} is independently hydrogen, halogen, ═O, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{7.2}-substituted or unsubstituted alkyl, R^{7.2}-substituted or unsubstituted heteroalkyl, R^{7.2}-substituted or unsubstituted cycloalkyl, R^{7.2}-substituted or unsubstituted heterocycloalkyl, R^{7.2}-substituted or unsubstituted aryl, or R^{7.2}-substituted or unsubstituted heteroaryl. R^{6.2} may be R^{7.2}-substituted or unsubstituted (e.g., C₁-C₂₀ or C₁-C₆) alkyl, R^{7.2}-substituted or unsubstituted (e.g., 2 to 20 membered or 2 to 6 membered) heteroalkyl, R^{7.2}-substituted or unsubstituted (e.g., C₃-C₈ or C₅-C₇) cycloalkyl, R^{7.2}-substituted or unsubstituted (e.g., 3 to 8 membered or 3 to 6 membered) heterocycloalkyl, R^{7.2}-substituted or unsubstituted (e.g., C₅-C₁₀ or C₅-C₆) aryl, or R^{7.2}-substituted or unsubstituted (e.g., 5 to 10 membered or 5 to 6 membered) heteroaryl. In embodiments, R^{6.2} is R^{7.2}-substituted or unsubstituted C₁-C₆ alkyl. In embodiments, R^{6.2} is R^{7.2}-substituted or unsubstituted C₁-C₅ alkyl. In embodiments, R^{6.2} is R^{7.2}-substituted or unsubstituted C₁-C₄ alkyl. In embodiments, R^{6.2} is R^{7.2}-substituted or unsubstituted C₁-C₃ alkyl. In embodiments, R^{6.2} is R^{7.2}-substituted C₁-C₆ alkyl. In embodiments, R^{6.2} is R^{7.2}-substituted C₁-C₅ alkyl. In embodiments, R^{6.2} is R^{7.2}-substituted C₁-C₄ alkyl. In embodiments, R^{6.2} is R^{7.2}-substituted C₁-C₃ alkyl. In embodiments, R^{6.2} is unsubstituted C₁-C₆ alkyl. In embodiments, R^{6.2} is unsubstituted C₁-C₅ alkyl. In embodiments, R^{6.2} is unsubstituted C₁-C₄ alkyl. In embodiments, R^{6.2} is unsubstituted C₁-C₃ alkyl. In embodiments, R^{6.2} is unsubstituted methyl.

R⁷, R^{7.1} and R^{7.2} are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. R⁷, R^{7.1} and R^{7.2} may be independently unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, the A2A receptor antagonist is a compound of formula:

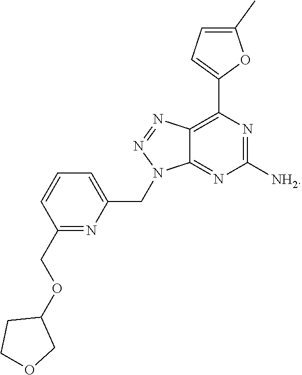 [SMILES: Cc1ccc(-c2nc(N)nc3c2nnn3Cc2cccc(COC3CCOC3)n2)o1]

In embodiments, the A2A receptor antagonist is a compound of formula:

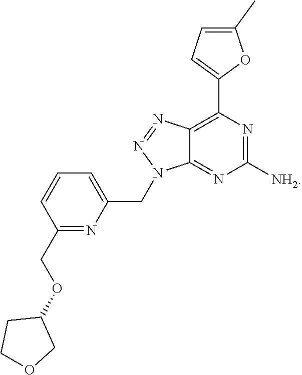

In embodiments, the A2A receptor antagonist is a compound of formula:

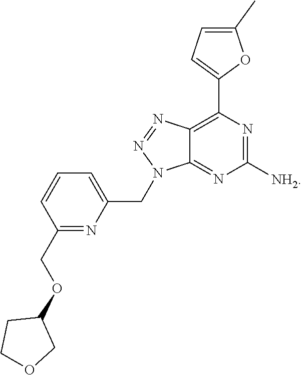

All compounds provided herein may optionally be provided as a pharmaceutically acceptable salt.

In embodiments, the compound is a purine receptor antagonist. In embodiments, the compound is an A_{2A} adenosine receptor antagonist or A_{2B} adenosine receptor antagonist. In embodiments, the compound is an A_{2A} adenosine receptor antagonist. In embodiments, the compound is A_{2B} adenosine receptor antagonist. In embodiments, the compound is any one of the compounds disclosed in U.S. Pat. Nos. 9,120,807, 8,450,328 or 8,354,415, which are hereby incorporated by reference and for all purposes. In embodiments, the A_{2A} adenosine receptor antagonist is a thienopyrimidine compound. In embodiments, the A_{2A} adenosine receptor antagonist is compound CPI-444. In embodiments, compound CPI-444 has the structure:

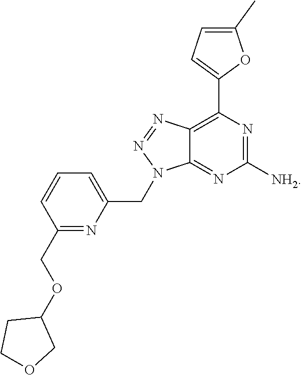 [SMILES: Cc1ccc(-c2nc(N)nc3c2nnn3Cc2cccc(COC3CCOC3)n2)o1]

In embodiments, compound CPI-444 has the structure:

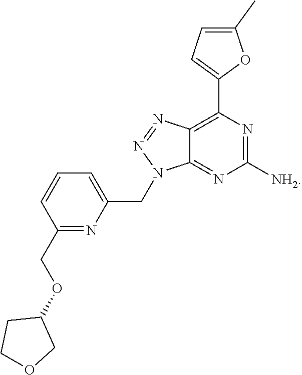

In embodiments, compound CPI-444 has the structure:

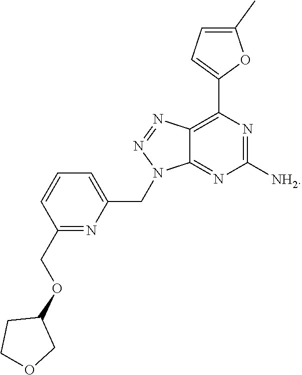

The term “A_{2A} adenosine receptor” as provided herein includes any of the recombinant or naturally-occurring forms of the A_{2A} adenosine receptor (ADORA2A) or variants or homologs thereof that maintain ADORA2A protein activity (e.g. within at least 50%, 80%, 90%, 95%, 96%, 97%, 98%, 99% or 100% activity compared to ADORA2A). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ADORA2A polypeptide. In embodiments, ADORA2A is the protein as identified by the NCBI sequence reference GI:5921992, homolog or functional fragment thereof.

The term “A_{2B} adenosine receptor” as provided herein includes any of the recombinant or naturally-occurring forms of the A_{2B} adenosine receptor (ADORA2B) or variants or homologs thereof that maintain ADORA2B protein activity (e.g. within at least 50%, 80%, 90%, 95%, 96%, 97%, 98%, 99% or 100% activity compared to ADORA2B). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ADORA2B polypeptide. In embodiments, ADORA2B is the protein as identified by the NCBI sequence reference GI:4501951, homolog or functional fragment thereof.

In embodiments, antibody and the second therapeutic agent are administered in a combined synergistic amount.

A “combined synergistic amount” as used herein refers to the sum of a first amount (e.g., an amount of humanized 1E9 antibody) and a second amount (e.g., an amount of A_{2A} adenosine receptor antagonist) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the humanized 1E9 antibody, an IgG1 antibody or anti-CD73 antibody provided herein, when used separately from the second therapeutic agent (e.g., A_{2A} adenosine receptor antagonist, CPI-444).

A “combined additive amount” as used herein refers to the sum of a first amount (e.g., an amount of an 1E9 antibody, a humanized 1E9 antibody, anti-CD73 antibody) and a second amount (e.g., an amount of A_{2A} adenosine receptor antagonist) that results in an additive effect (i.e. an effect equal to the sum of the effects). Therefore, the terms “additive”, “combined additive amount”, and “additive therapeutic effect” which are used herein interchangeably, refer to a measured effect of compounds administered in combination where the measured effect is equal to the sum of the individual effects of each of the compounds administered alone as a single agent.

In the provided methods of treatment, additional therapeutic agents can be used that are suitable to the disease (e.g., cancer) being treated. Thus, in some embodiments, the provided methods of treatment further include administering a second therapeutic agent to the subject. Suitable additional therapeutic agents include, but are not limited to analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

According to the methods provided herein, the subject is administered an effective amount of one or more of the therapeutic agents provided herein (i.e. a humanized 1E9 antibody or a humanized IgG1 or IgG4 antibody in combination with, for example, a compound or a second humanized antibody). The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

When administered in methods to treat a disease (e.g., cancer), pharmaceutical compositions will contain an amount of active humanized antibody effective to achieve the desired result, e.g., modulating the activity of a target molecule (e.g., CD73), and/or reducing, eliminating, or slowing the progression of disease symptoms (e.g., cancer). Determination of a therapeutically effective amount of a humanized antibody provided herein is well within the capabilities of those skilled in the art, especially in light of the detailed disclosure herein.

Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids such as glycine, carbohydrates, chelating agents, sugars, and other standard ingredients known to those skilled in the art (Remington's Pharmaceutical Science 16^{th} edition, Osol, A. Ed. 1980). The mAb is typically present at a concentration of 0.1-100 mg/ml, e.g., 1-10 mg/ml or 10-50 mg/ml, for example 5, 10, 20, 30, 40, 50 or 60 mg/ml.

A pharmaceutical composition including a humanized antibody as described herein can be administered by a variety of methods known in the art. The route and/or mode of administration vary depending upon the desired results. In embodiments, administration is intravenous, intramuscular, intraperitoneal, or subcutaneous, or administered proximal to the site of the target. Pharmaceutically acceptable excipients can be suitable for intravenous, intramuscular, subcutaneous, parenteral, spinal or epidermal administration (e.g., by injection or infusion).

Pharmaceutical compositions of the humanized antibody can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the humanized antibody is employed in the pharmaceutical compositions of the invention. The humanized antibodies provided can be formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It may be advantageous to formulate the humanized antibodies in combination with other therapies or agents. It can be advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of humanized antibody calculated to produce the desired therapeutic effect in association with the required pharmaceutical excipient.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular antibody being employed, the duration of the treatment, other drugs, compounds and/or materials used in combination with the particular compositions employed, the age, sex, weight, condition, general health and prior medical history of the patient being treated, and like factors.

A physician or veterinarian can start doses of the humanized antibodies of the invention employed in the pharmaceutical composition at levels lower than that required to achieve the desired therapeutic effect and gradually increase the dosage until the desired effect is achieved. In general, effective doses of the compositions of the present invention vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months.

The antibody provided herein can be administered on multiple occasions. Intervals between single dosages can be weekly, monthly or yearly. Intervals can also be irregular as indicated by measuring blood levels of the humanized antibody in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, humanized antibodies show longer half-life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

### Example 1: A Novel CD73-Blocking Antibody Reduces Production of Immunosuppressive Adenosine and Restores T Cell Function

Adenosine is present at high concentrations in the tumor microenvironment and is immunosuppressive acting on multiple cell types, including suppression of effector T cells. CD73, an ectonucleotidase that converts AMP to adenosine, is expressed on a subset of B and T cells and is a major source of extracellular adenosine. Elevated CD73 expression has been observed in multiple tumor types and is prognostic in triple negative breast cancer supporting a role for CD73 in tumor progression¹. Inhibiting catalytic activity of CD73 is an attractive therapeutic strategy to reduce adenosine-mediated suppression of tumor immunity.

Applicants developed two types of humanized monoclonal anti-CD73 antibodies. CPX-006 inhibits CD73 catalytic activity by competing directly with AMP for the active site with an affinity of 0.64 nM. CPX-016 is similar to anti-CD73 antibodies described by others², and inhibits CD73 activity allosterically by binding to a distal site. This was demonstrated using CD73 expressing cells incubated with APCP, a non-hydrolyzable analog of AMP. APCP competes with CPX-006 for binding to CD73 in contrast to CPX-016. The CPX-016 mechanism requires higher order complexes and results in loss of inhibition at high CPX-016/CD73 ratios. In contrast, CPX-006 reduces catalytic activity completely in cell based assays to levels seen when CD73 gene is deleted using CRISPR technology and inhibition was unaffected at high CPX-006/CD73 ratios.

In a functional assay, CPX-006 inhibited immune suppression of T cell function induced by exposure of human PBMCs to AMP through direct inhibition of catalytic activity with a mean EC₅₀ of 137 nM (interferon gamma production) and 189 nM (T cell proliferation) (n=12 donors).

Applicants analyzed the prevalence of CD73 expression across tumor histologies by immunohistochemistry including renal cell carcinoma (RCC, n=62), non small cell lung cancer (NSCLC, n=68), melanoma (n=68) and breast cancer (n=94). CD73 was found to be heterogeneously expressed on tumor cells, immune cells and other stromal elements within each of the histologies examined. Expression on tumor cell only was found in 15% of melanoma and 14% of squamous NSCLC cases. In contrast, tumor cell staining was found in a significant fraction of the adenocarcinoma sub-type of NSCLC (55%). Stromal cell staining to varying degrees was seen in all tumor tissues.

In summary, Applicants have generated a therapeutic antibody, CPX-006, that utilizes a novel mechanism of binding to the CD73 active site to completely inhibit CD73 enzymatic activity and restore T cell function. The finding of CD73 expression on tumor cells and stromal cells in a variety of tumors suggest this as a potential new target for immunotherapy of these malignancies.

### Example 2: Methods

APCP experiment indicating epitope. MDA-MB-231 cells were preincubated with APCP (Sigma-Aldrich) over a range of concentrations for 30 minutes at 37° C. prior to staining with unlabeled CPX-006 or CPX-016. Cells were washed and stained with PE anti-human secondary (Jackson Immunoresearch) and analyzed using the CytoFLEX flow cytometer (Beckman Coulter).

In vitro T cell assay for proliferation and cytokine secretion. LRS chambers containing white blood cell concentrate after plateletpheresis were obtained from healthy donors through the Stanford Blood Center. Peripheral Blood Mononuclear Cells (PBMC) were isolated by Ficoll Paque gradient according to manufacturer's instructions (GE Healthcare) and purified cells were labeled with Cell Trace Violet according to manufacturer's instructions (Thermo Fisher). Labeled PBMC were reconstituted in RPMI 1640 media with 10% FBS and 200 units/mL IL-2 and seeded at a density of 6×10e5 cells/well in 96 well round bottom tissue culture plates. Anti-CD3 (clone HIT3a) and anti-CD28 (clone CD28.2) were added to each well at a final concentration of 1 μg/mL to stimulate T cell proliferation. CPX-006, CPI-444, or human IgG1 isotype control (Sigma-Aldrich) was added to each well at the final concentrations indicated in the figure legends. AMP (Sigma-Aldrich) was added to each well at final concentration of 3 mM and cells were incubated for 4 days at 37° C., 5% CO₂.

For flow cytometry analysis of T cell proliferation, duplicate wells were pooled and stained with PerCP-Cy5.5 anti-CD3 (BD). After staining, cell were washed and fixed with 4% PFA and analyzed using the CytoFLEX flow cytometer (Beckman Coulter). Data were analyzed with FlowJo software and proliferating CD3+ T cells were identified by gating relative to control cells not stimulated with anti-CD3/28 or AMP. To compile data derived from multiple donors, experimental values were normalized to the value derived from cells stimulated with anti-CD3/28 in the absence of AMP for each donor.

To evaluate cytokine secretion, experimental cultures were set up as described above except that cells were seeded at a density of 1.5×10e5 cells/well and labeling with Cell Trace Violet was omitted. Interferon gamma levels were measured using the IFN-γ human AlphaLISA Detection assay kit according to manufacturer's recommendations (Perkin Elmer). Data from multiple donors were compiled as described above.

CD73 score determination and correlation to T cell activity. CD73 protein expression was measured for each donor analyzed in the T cell proliferation assay. Frozen PBMC were thawed and stained with PE anti-CD73, clone AD2 (BD). After staining, cells were washed and fixed with 4% PFA and analyzed using the CytoFLEX flow cytometer (Beckman Coulter). Data were analyzed with FlowJo software and gating relative to fluorescence minus one control identified CD73+ cells. CD73 score was calculated by multiplying the % CD73 positive cells by the mean fluorescence intensity (MFI) of the same population. In order to determine the correlation of CD73 score to antibody-mediated restoration of T cell proliferation in the presence of AMP, values were plotted on an XY plot and linear regression analysis was performed.

Immunohistochemistry. Tissue microarray slides were obtained from US Biomax Inc and were stained with hematoxylin and eosin and CD73 antibody at Ventana Medical Systems Inc. Immunostaining was performed on the VMSI Benchmark Ultra instrument using Optiview polymer DAB detection system. The antibody used was rabbit monoclonal D7F9A (Cell Signaling Technology). Two pathologists evaluated all stained slides. For scoring of epithelial cells, H scores were determined for membrane and cytoplasm cellular compartments using the following formula: [1*(percentage of cells staining at 1+intensity)]+[2*(percentage of cells staining at 2+intensity)]+[3*(percentage of cells staining at 3+intensity)]=H score.

Inhibition of CD73 activity by CPX-006 in engineered cell lines. MDA-MB-231 cells were engineered to modulate CD73 expression using lentiviral shRNA particles according to manufacturer's instructions (Origene). For complete knock out of CD73 expression, CRISPR technology was employed with plasmids obtained from Santa Cruz Biotechnology according to manufacturer's instructions. Knockdown was confirmed by flow cytometry analysis of CD73 protein levels using AlexaFluor647-conjugated CPX-006.

To assess the ability of anti-CD73 antibodies to inhibit CD73 activity on these engineered cell lines, cell were incubated with antibodies over a range of concentrations prior to addition of 250 μM AMP. Phosphate levels were measured in the cell culture supernatant using the Sensolyte Malachite Green assay kit.

### Example 3: CPX-006 and CPX-016 Bind to CD73 and Reduce Adenosine Production

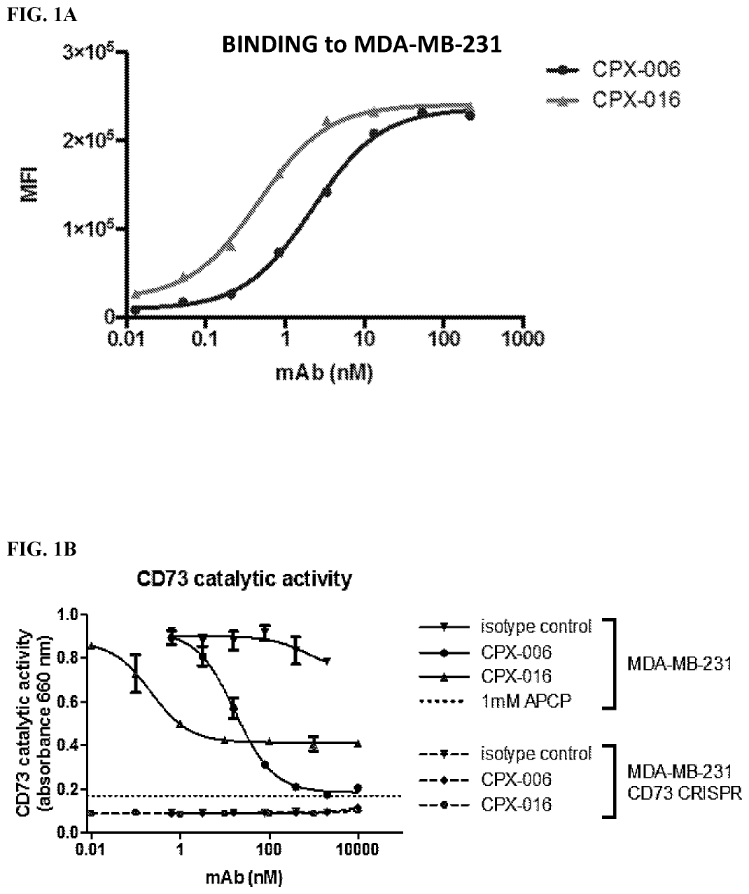

MDA-MB-231 cells were incubated with CPX-006 or CPX-016 over a range of concentrations and antibody binding was detected with a PE-conjugated secondary antibody and flow cytometry analysis. The mean fluorescence intensity (MFI) of PE signal is reported (FIG. 1A). MDA-MB-231 parental cells or CD73 CRISPR knockout cells were incubated with isotype control, CPX-006, CPX-016, or APCP, a small molecule inhibitor of CD73 enzymatic activity, prior to addition of 250 μM AMP. Phosphate levels were measured in the cell culture supernatant using the Sensolyte Malachite Green phosphate assay kit (FIG. 1B).

### Example 4: CPX-006 and CPX-016 Bind to Distinct Epitopes on CD73

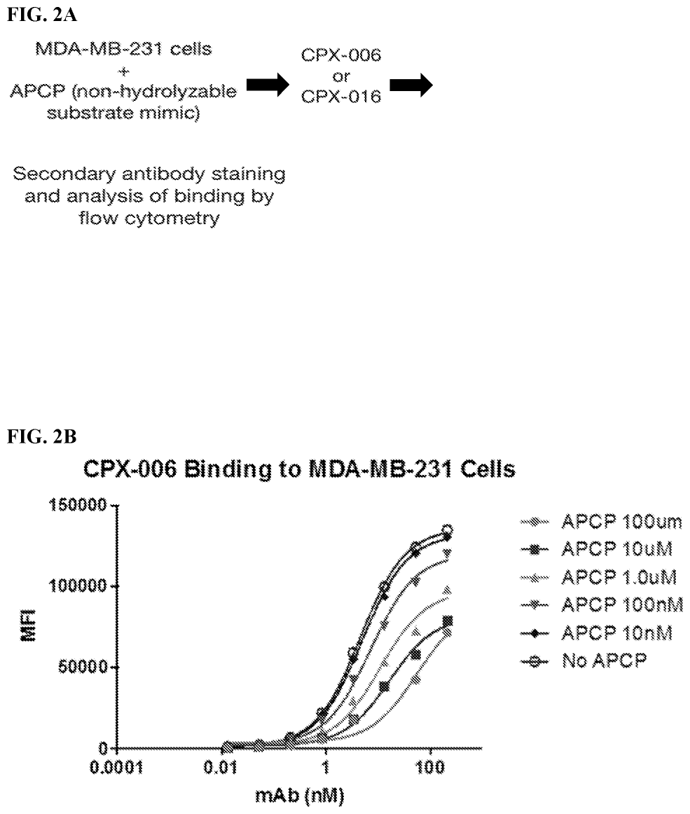

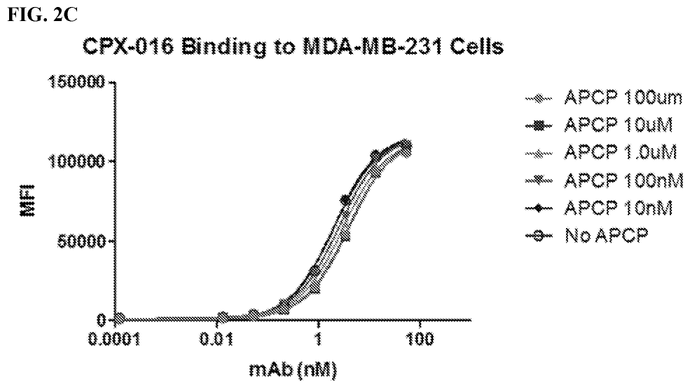

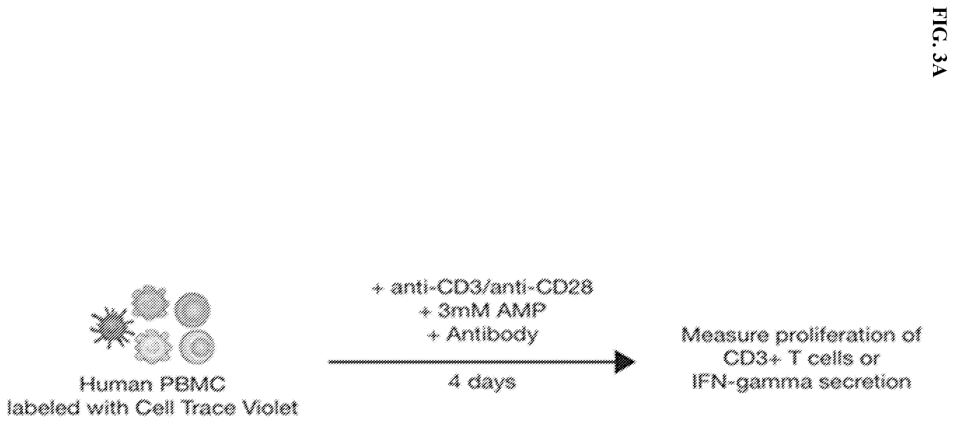

MDA-MB-231 cells were pre-incubated with APCP, a non-hydrolyzable substrate mimic for CD73. Cells were subsequently incubated with a titration of CPX-006 or CPX-016 prior to staining with PE-conjugated anti-human secondary antibody. Antibody binding was assessed by flow cytometry and mean fluorescence intensity (MFI) for PE was determined (FIG. 2A-2B).

### Example 5: CPX-006 Reverses Suppressive Effects of AMP on T Cell Activity

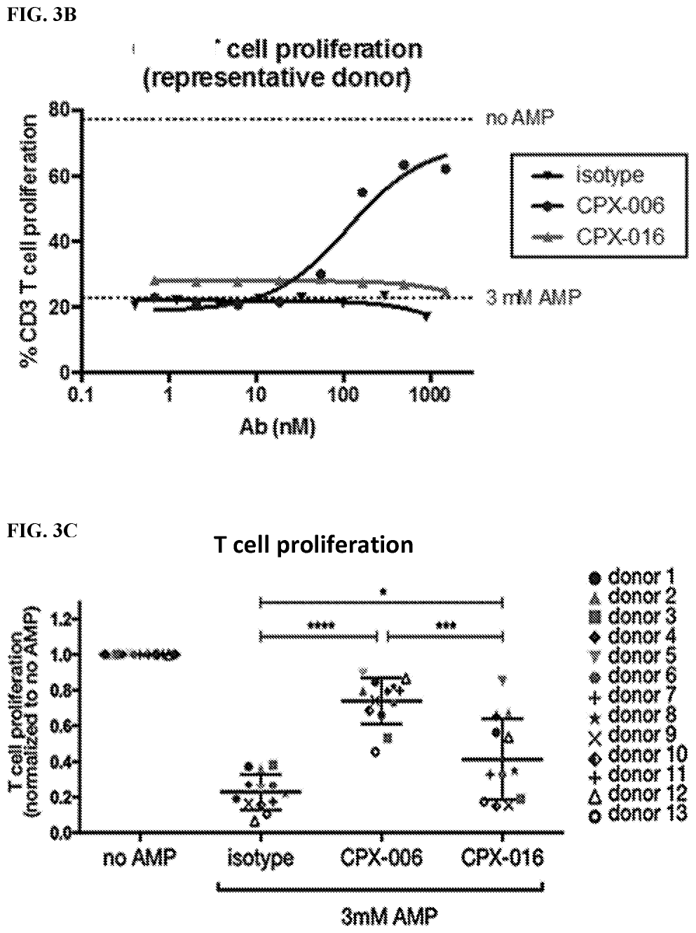

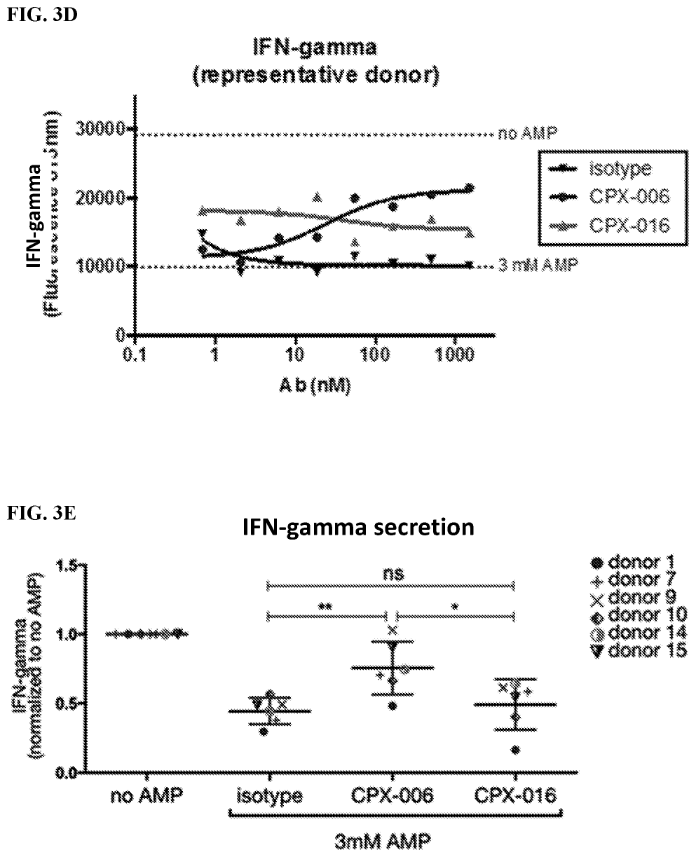

FIG. 3A shows a schematic of the experimental design. PBMC were isolated from healthy donors and labeled with Cell Trace Violet prior to culture with 1 μg/mL anti-CD3 and anti-CD28, 200 units/mL IL-2, 3 mM AMP, and antibody treatment as indicated. After 4 days in culture, IFN-gamma levels in the supernatant were measured by AlphaLISA. Proliferation of CD3+ T cells was followed by flow cytometry analysis of Cell Trace Violet dilution and was defined by gating relative to unstimulated PBMC. FIG. 3B shows T cell proliferation for a representative donor. FIG. 3C shows T cell proliferation for 13 donors treated with 500 nM CPX-006 or CPX-016 or 890 nM isotype control. In FIG. 3D is IFN-gamma production (reported as raw AlphaLISA values) for a representative donor. FIG. 3E shows IFN-gamma production for 6 donors treated with 500 nM CPX-006 or CPX-016 or 890 nM isotype control.

### Example 6: CPX-006 Reverses Suppressive Effects of AMP Across a Broad Range of CD73 Expression

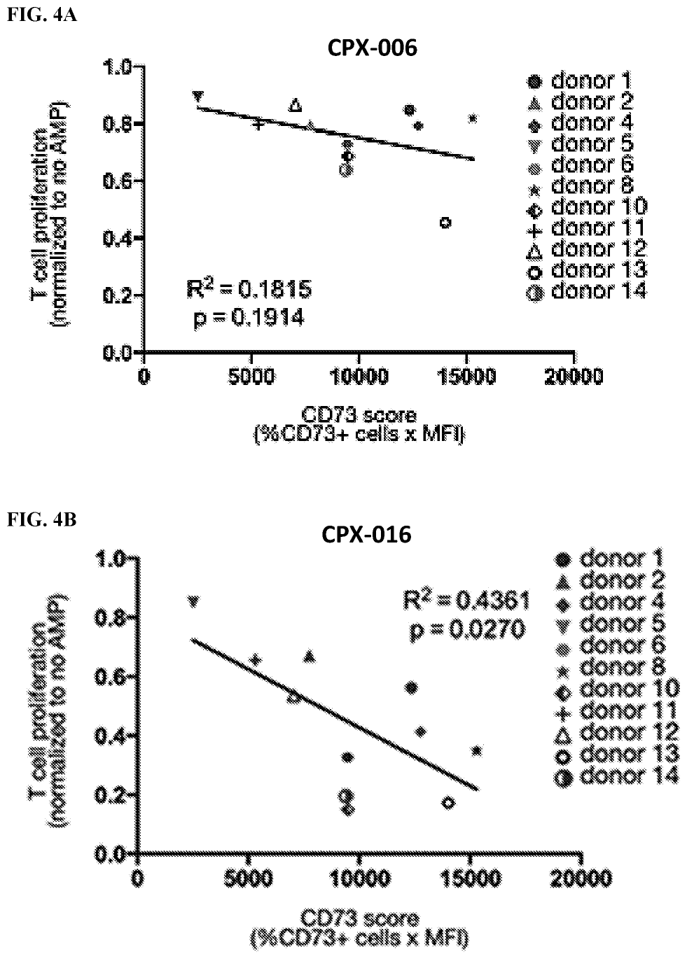

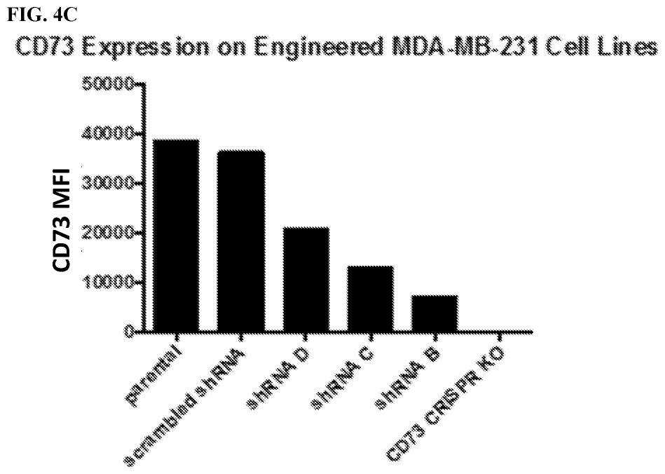

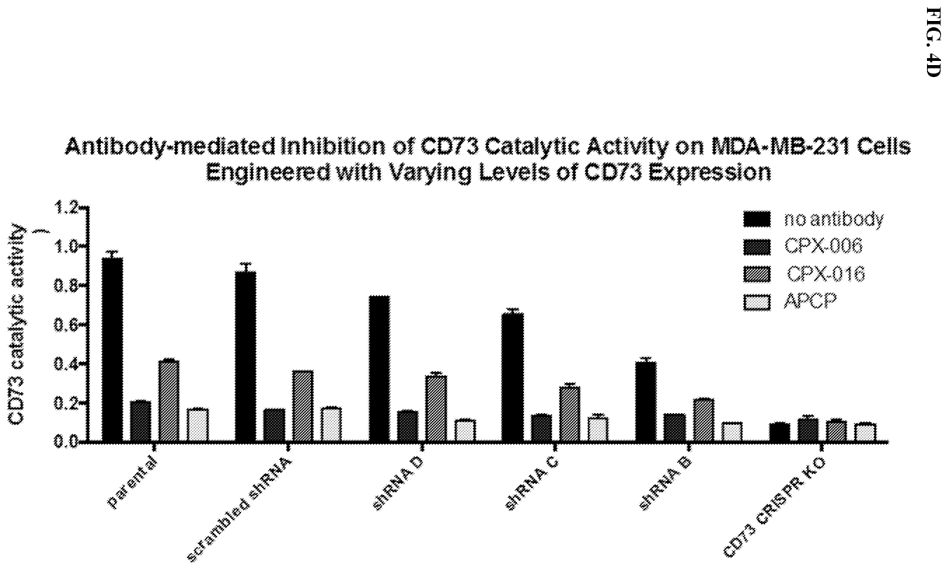

CD73 expression levels in each donor were determined by flow cytometry and were plotted as a function of the effect of CPX-006 (FIG. 4A) or CPX-016 (FIG. 4B) on restoring T cell proliferation in the presence of AMP as reported in FIG. 3C.

MDA-MB-231 cells were engineered to reduce or eliminate CD73 expression via shRNA or CRISPR, respectively. CD73 protein levels were measured by flow cytometry analysis (FIG. 4C). CD73 catalytic activity was measured for each cell line in the presence of 1 μM CPX-006 or CPX-016 or 1 mM APCP (FIG. 4D) as described in FIG. 1B.

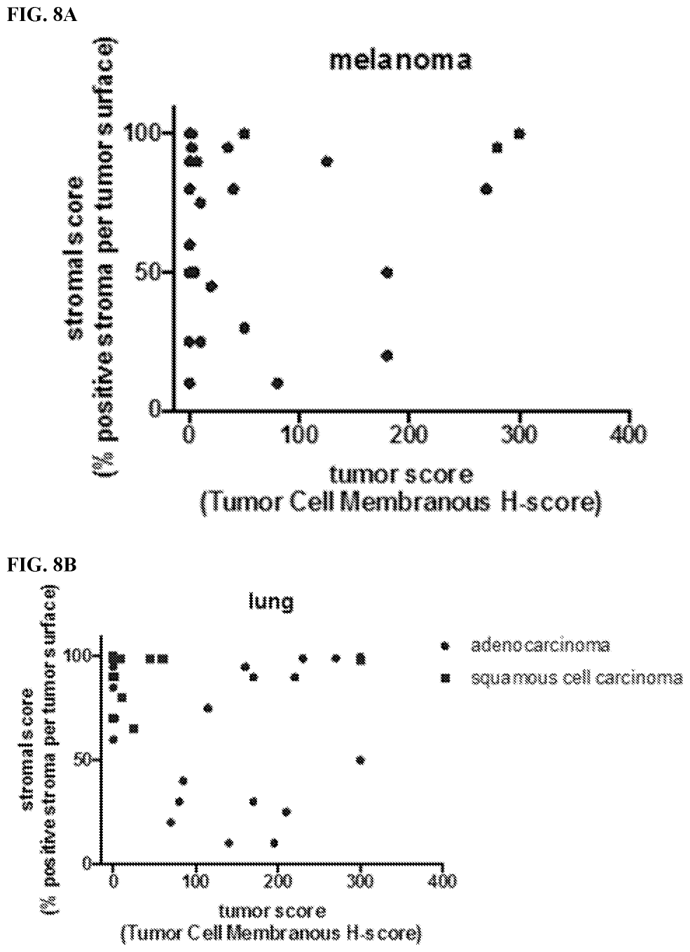

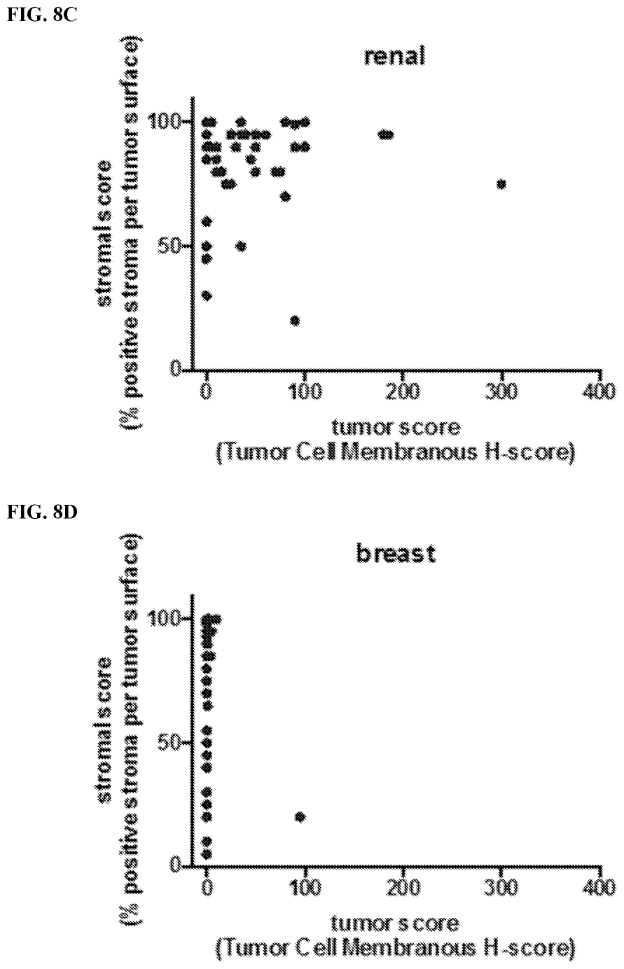

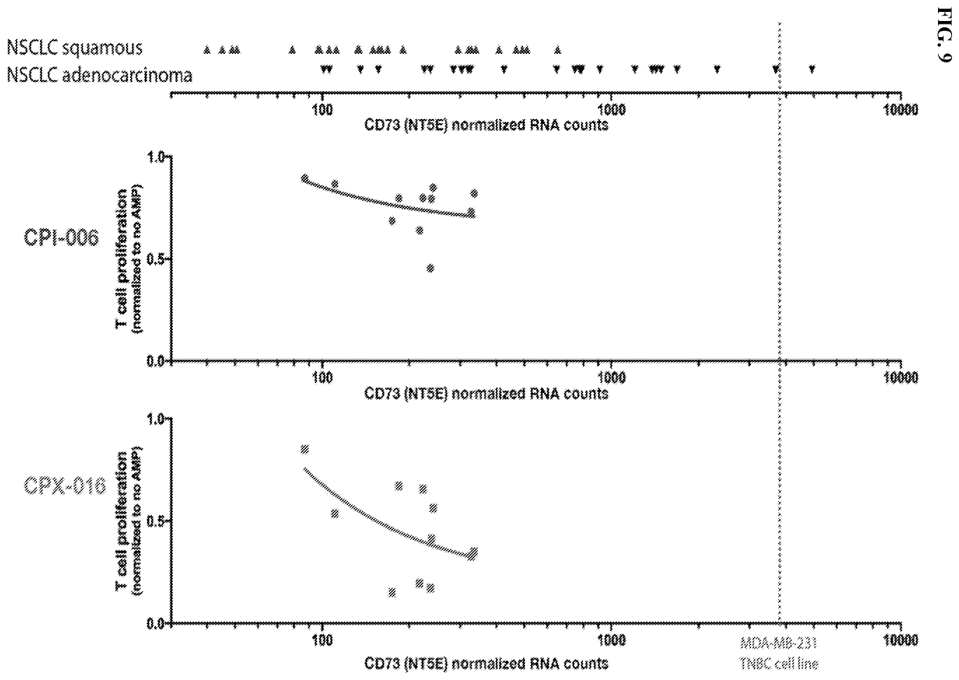

All tumor samples from major IO indications expressed CD73. Expression was predominantly in the stroma. FIGS. 8A-8D are scatter plots comparing the percentage of CD73 positive stroma per tumor surface (stromal score) with the tumor cell membranous H-score (tumor score) for melanoma (FIG. 8A), lung cancer (adenocarcinoma or squamous cell carcinoma; FIG. 8B), renal cancer (FIG. 8C), and breast cancer tissue samples (FIG. 8D).

### Example 7: CD73 is Highly Expressed in Multiple Tumor Types

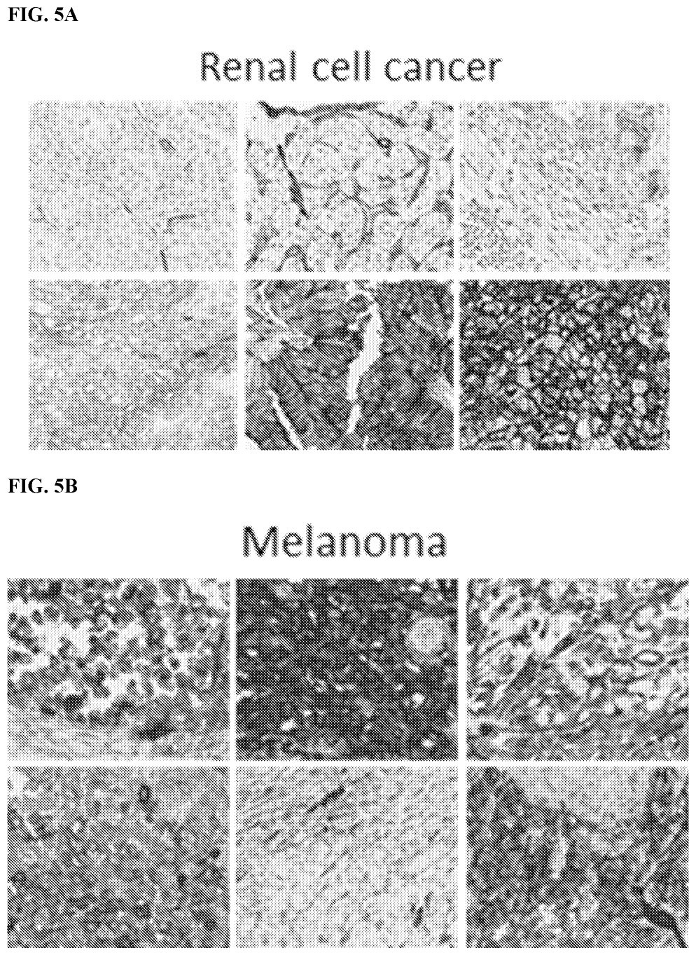

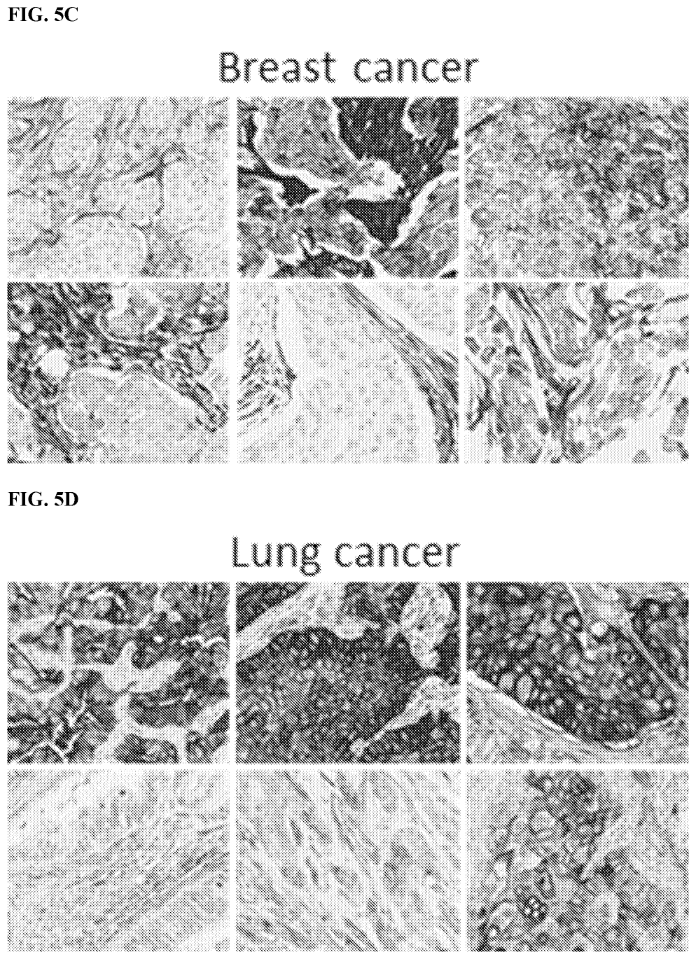

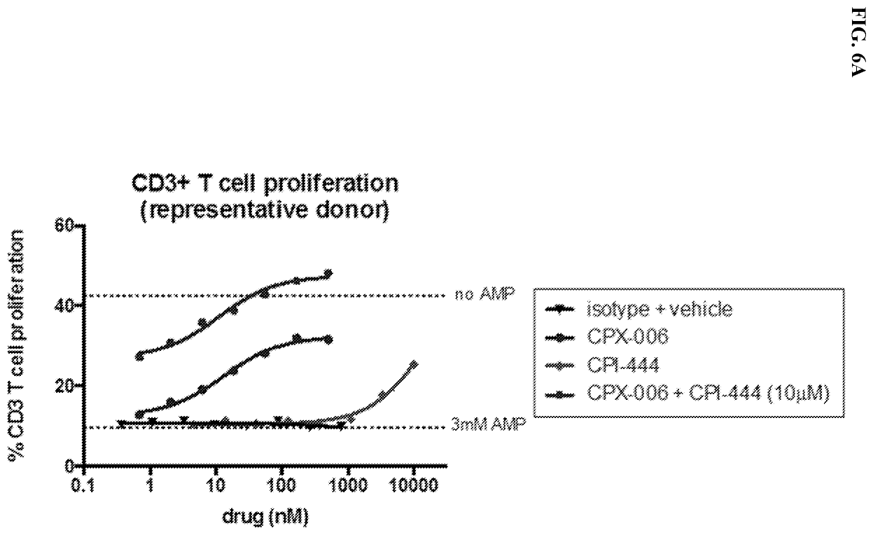

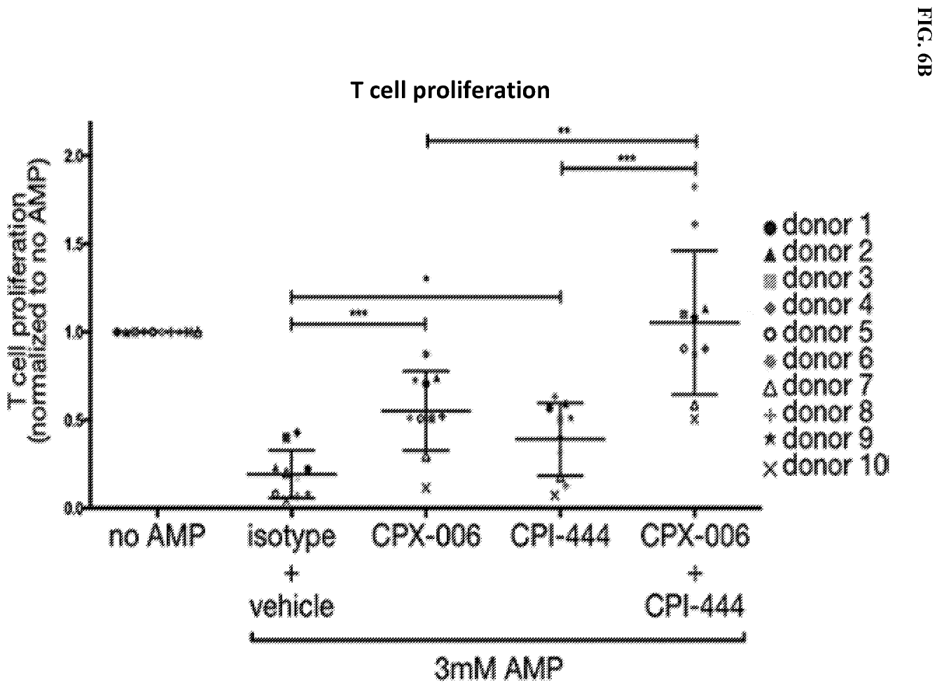

CD73 expression was analyzed by immunohistochemical staining of tissues from renal cell cancer (n=62), NSCLC (n=68), melanoma (n=68), and breast cancer (n=94). Six representative tissues are shown from each histology (FIGS. 5A-5D).

### Example 8: CPX-006 Augments CPI-444 to Reverse AMP-mediated Suppression of T Cell Proliferation

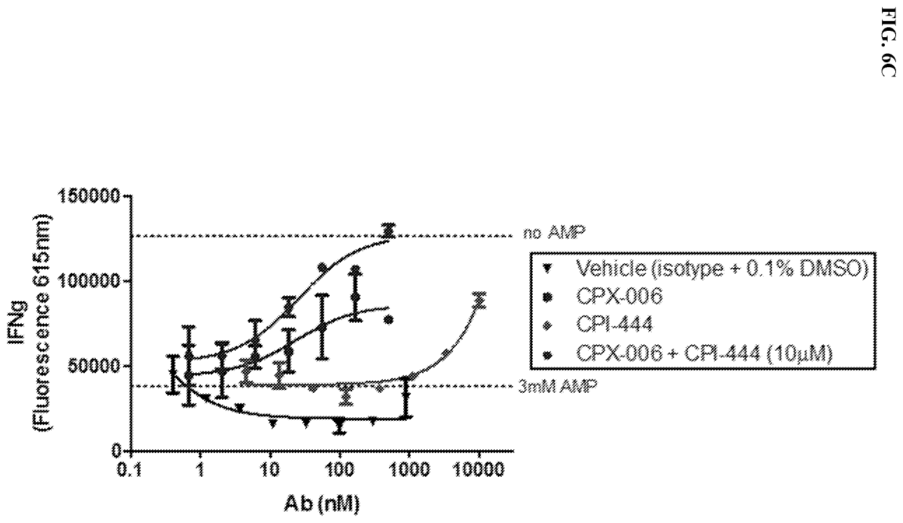

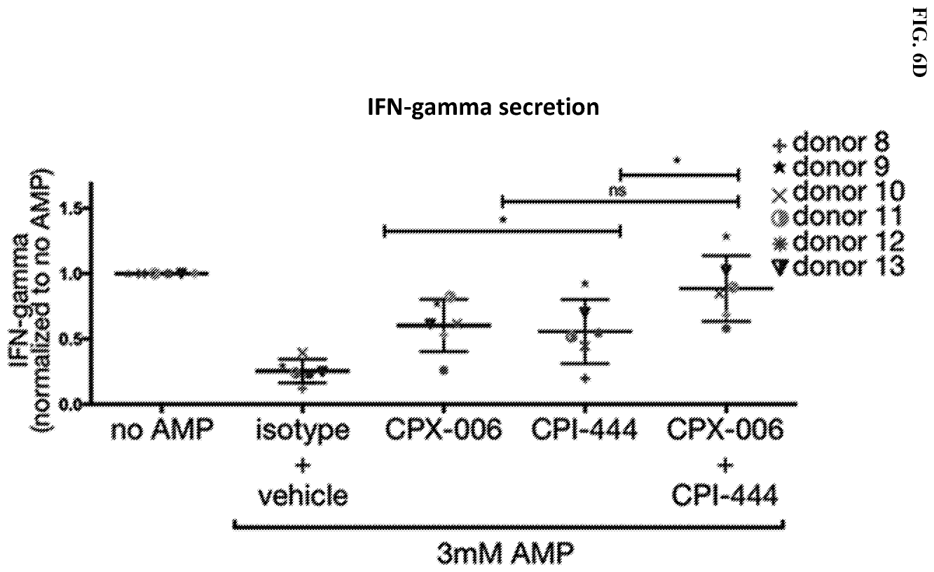

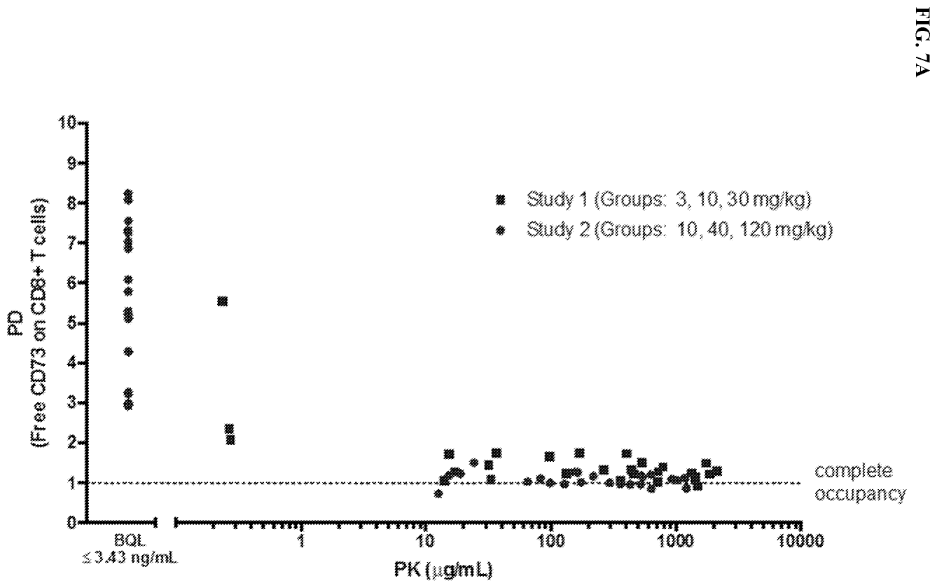

Effects of CPX-006 and CPI-444 on T cell proliferation were measured as described in FIG. 3A. FIG. 6A shows the percentage of CD3+ T cell proliferation in a representative donor treated with isotype and vehicle, CPX-006, CPI-444, or CPX-006 and CPI-444 (10 μM). Dotted lines indicate percentage of CD3+ T cell proliferation in the presence of no AMP or 3 mM AMP.

FIG. 6B shows T cell proliferation for 10 donors treated with 500 nM CPX-006, 10 μM CPI-444, 500 nM CPX-006+10 μM CPI-444, or vehicle (890 nM isotype control+0.1% DMSO).

### Example 9: CPX-006 Augments CPI-444 to Reverse AMP-Mediated Suppression of IFN-Gamma Secretion

Effects of CPX-006 and CPI-444 on IFN-gamma secretion were measured as described in FIG. 3A. FIG. 6C shows IFNg secretion as evidence by fluorescence intensity in response to treatment with vehicle (isotype and 0.1% DMSO), CPX-006, CPI-444, or CPX-006 and CPI-444 (10 μM). Dotted lines indicate IFN-gamma secretion as evidence by fluorescence intensity in the presence of no AMP or 3 mM AMP.

FIG. 6D shows IFN-gamma production (reported as raw AlphaLISA values) for 6 donors treated with 500 nM CPX-006, 10 μM CPI-444, 500 nM CPX-006+10 μM CPI-444, or vehicle (890 nM isotype control+0.1% DMSO).

### Example 10: Complete Occupancy Achieved in Repeat-Dose Tox Study with No Observed Toxicities

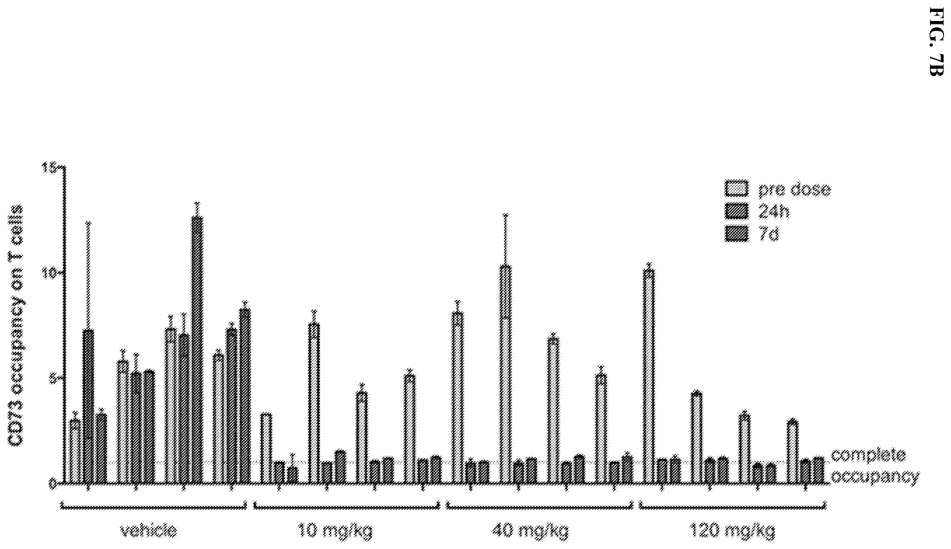

Cynomolgus monkeys were dosed weekly for five consecutive weeks. No changes were observed in clinical chemistry, gross, pathology, organ weights, or histopathology. FIG. 7A shows PD (Free CD73 on CD8+ T cells) as a function of PK (μg/mL) for two studies. Study 1 (Groups: 3, 10, 30 mg/kg) and Study 2 (Groups: 10, 40, 120 mg/kg) both demonstrated complete occupancy with repeat dosing (FIG. 7A).

Cynomolgus monkeys were dosed weekly for five consecutive weeks at 10, 40, or 120 mg·kg. No changes were observed in clinical chemistry, gross pathology, organ weights, or histophathology. FIG. 7B shows CD73 occupancy determined by measuring ex vivo staining of CD8+ T cells with Alexa-Fluor 647 labeled CPX-006. The ratio of AF647 staining on CD73+/CD73− cells is reported. Complete CD73 coverage was achieved with CPX-006 at all doses treated.

### EMBODIMENTS

Embodiment 1. A method of treating cancer in a subject in need thereof, the method including administering to the subject a therapeutically effective amount of a 1E9 antibody, wherein the subject expresses an elevated level of CD73 relative to a standard control, and wherein the 1E9 antibody includes (i) a mouse CDR L1 as set forth in SEQ ID NO:1, a mouse CDR L2 as set forth in SEQ ID NO:2, a mouse CDR L3 as set forth in SEQ ID NO:3; and (ii) a mouse CDR H1 as set forth in SEQ ID NO:4, a mouse CDR H2 as set forth in SEQ ID NO:5, and a mouse CDR H3 as set forth in SEQ ID NO:6.

Embodiment 2. The method of embodiment 1, wherein the 1E9 antibody is a humanized 1E9 antibody.

Embodiment 3. The method of embodiment 2, wherein the humanized 1E9 antibody includes a humanized light chain variable region and a humanized heavy chain variable region, wherein the humanized light chain variable region comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87; and wherein the humanized heavy chain variable region comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

Embodiment 4. The method of one of embodiments 1-3, wherein said elevated level of CD73 has an H-score of at least 150.

Embodiment 5. The method of one of embodiments 1-4, wherein the effective amount of the antibody is administered at a 1:1 ration relative to said elevated level of CD73.

Embodiment 6. The method of one of embodiments 1-5, wherein the antibody is administered at a half maximal effective concentration (EC₅₀) of at least 100 nM.

Embodiment 7. The method of one of embodiments 1-6, wherein the antibody is administered at an EC₅₀ of about 137 nM.

Embodiment 8. The method of one of embodiments 1-7, wherein the antibody is administered at an EC₅₀ of about 189 nM.

Embodiment 9. The method of one of embodiments 1-8, wherein the therapeutically effective amount is about 3 mg/kg, 10 mg/kg, 30 mg/kg, 40 mg/kg, or 120 mg/kg.

Embodiment 10. The method of one of embodiments 3-9, wherein the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and wherein the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

Embodiment 11. The method of one of embodiments 3-10, wherein the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and wherein the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

Embodiment 12. The method of one of embodiments 3-9, wherein the humanized light chain variable region comprises a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and wherein the humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

Embodiment 13. The method of one of embodiments 3-9 or 12, wherein the humanized light chain variable region comprises a valine at a position corresponding to Kabat position 2, a methionine at a position corresponding to Kabat position 4, a leucine at a position corresponding to Kabat position 9, a proline at a position corresponding to Kabat position 12 and a proline at a position corresponding to Kabat position 18; and wherein the humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85 and a methionine at a position corresponding to Kabat position 89.

Embodiment 14. The method of one of embodiments 3-9, wherein the humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and wherein the humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

Embodiment 15. The method of one of embodiments 3-9 or 14, wherein the humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and wherein the humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

Embodiment 16. The method of one of embodiments 3-9, wherein the humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

Embodiment 17. The method of one of embodiments 3-9 or 16, wherein the humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

Embodiment 18. The method of one of embodiments 3-11, 16 or 17, wherein the humanized heavy chain variable region comprises the sequence of SEQ ID NO:7.

Embodiment 19. The method of one of embodiments 3-11 or 16-18, wherein the humanized light chain variable region comprises the sequence of SEQ ID NO:37.

Embodiment 20. A method of treating cancer in a subject in need thereof, the method including administering to the subject a therapeutically effective amount of a humanized 1E9 antibody, wherein the subject expresses an elevated level of CD73 relative to a standard control and wherein the humanized 1E9 antibody includes a humanized light chain variable region and a humanized heavy chain variable region, wherein the humanized heavy chain variable region includes the sequence of SEQ ID NO:7.

Embodiment 21. The method of embodiment 20, wherein the humanized light chain variable region includes the sequence of SEQ ID NO:37.

Embodiment 22. The method of one of embodiments 1-21, wherein the antibody is an IgG.

Embodiment 23. The method of one of embodiments 1-22, wherein the antibody is an IgG1.

Embodiment 24. The method of one of embodiments 1-22, wherein the antibody is an IgG4.

Embodiment 25. The method of one of embodiments 1-21, wherein the antibody is a Fab′ fragment.

Embodiment 26. The method of one of embodiments 1-21, wherein the antibody is a single chain antibody (scFv).

Embodiment 27. The method of one of embodiments 1-26, wherein the antibody is capable of binding a CD73 antigen with an equilibrium dissociation constant (K_{D}) from about 0.3 to about 25 nM.

Embodiment 28. The method of one of embodiments 1-27, wherein the antibody is capable of binding a CD73 antigen with an equilibrium dissociation constant (K_{D}) of about 0.64 nM.

Embodiment 29. The method of one of embodiments 1-28, wherein the antibody is capable of binding a CD73 antigen at a pH of less than about 7.5.

Embodiment 30. The method of one of embodiments 1-29, wherein the antibody is capable of binding a CD73 antigen at a pH from about 6.0 to about 7.0.

Embodiment 31. The method of one of embodiments 1-30, wherein the antibody is capable of binding a CD73 antigen at a pH of about 6.3.

Embodiment 32. The method of one of embodiments 1-24 or 27-31, wherein the antibody further includes a glutamine at a position corresponding to Kabat position 297.

Embodiment 33. The method of one of embodiments 1-32, wherein the antibody is bound to a CD73 antigen.

Embodiment 34. The method of embodiment 33, wherein said CD73 antigen forms part of a cell.

Embodiment 35. The method of embodiment 34, wherein said cell is a lymphoid cell.

Embodiment 36. The method of embodiment 34, wherein said cell is a T cell.

Embodiment 37. The method of embodiment 34, wherein said cell is a cancer cell.

Embodiment 38. A method of treating cancer in a subject in need thereof, the method including administering to the subject a therapeutically effective amount of a humanized IgG1 antibody, wherein the subject expresses an elevated level of CD73 relative to a standard control and wherein the humanized IgG1 antibody includes a humanized light chain variable region and a humanized heavy chain variable region, wherein the humanized light chain variable region includes a mouse CDR L1 as set forth in SEQ ID NO:1, a mouse CDR L2 as set forth in SEQ ID NO:2, a mouse CDR L3 as set forth in SEQ ID NO:3; and wherein the humanized heavy chain variable region includes a mouse CDR H1 as set forth in SEQ ID NO:4, a mouse CDR H2 as set forth in SEQ ID NO:5, and a mouse CDR H3 as set forth in SEQ ID NO:6.

Embodiment 39. The method of embodiment 38, wherein the humanized light chain variable region further comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85 or a phenylalanine at a position corresponding to Kabat position 87.

Embodiment 40. The method of embodiment 38 or 39, wherein the humanized heavy chain variable region further comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 or a glutamic acid at a position corresponding to Kabat position 81.

Embodiment 41. The method of one of embodiments 38-40, wherein the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and wherein the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

Embodiment 42. The method of one of embodiments 38-41, wherein the humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

Embodiment 43. The method of one of embodiments 38-42, wherein the humanized heavy chain variable region comprises the sequence of SEQ ID NO:7.

Embodiment 44. The method of one of embodiments 38-43, wherein the humanized light chain variable region comprises the sequence of SEQ ID NO:37.

Embodiment 45. The method of one of embodiments 38-44, wherein the antibody further comprises a glutamine at a position corresponding to Kabat position 297.

Embodiment 46. The method of one of embodiments 38-45, wherein the antibody is bound to a CD73 antigen.

Embodiment 47. The method of embodiment 46, wherein the CD73 antigen forms part of a cell.

Embodiment 48. The method of embodiment 47, wherein said cell is a T cell.

Embodiment 49. The method of embodiment 47, wherein said cell is a cancer cell.

Embodiment 50. A method of treating cancer in a subject in need thereof, the method including administering to the subject a therapeutically effective amount of an anti-CD73 antibody, wherein the subject expresses an elevated level of CD73 relative to a standard control and wherein the anti-CD73 antibody binds the same epitope as a 1E9 antibody.

Embodiment 51. The method of embodiment 50, wherein the 1E9 antibody includes a humanized light chain variable region comprising a mouse CDR L1, mouse CDR L2, or mouse CDR L3 and a humanized heavy chain variable region comprising a mouse CDR H1, mouse CDR H2, or mouse CDR H3.

Embodiment 52. The method of embodiment 51, wherein the humanized light chain variable region further comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85 or a phenylalanine at a position corresponding to Kabat position 87.

Embodiment 53. The method of embodiment 51 or 52, wherein the humanized heavy chain variable region further comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 or a glutamic acid at a position corresponding to Kabat position 81.

Embodiment 54. The method of one of embodiments 51-53, wherein the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and wherein the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

Embodiment 55. The method of one of embodiments 51-54, wherein the humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

Embodiment 56. The method of one of embodiments 51-55, wherein the humanized heavy chain variable region comprises the sequence of SEQ ID NO:7.

Embodiment 57. The method of one of embodiments 51-56, wherein the humanized light chain variable region comprises the sequence of SEQ ID NO:37.

Embodiment 58. The method of one of embodiments 51-57, wherein the antibody further comprises a glutamine at a position corresponding to Kabat position 297.

Embodiment 59. The method of one of embodiments 51-58, wherein the antibody is bound to a CD73 antigen.

Embodiment 60. The method of embodiment 59, wherein the CD73 antigen forms part of a cell.

Embodiment 61. The method of embodiment 60, wherein said cell is a T cell.

Embodiment 62. The method of embodiment 60, wherein said cell is a cancer cell.

Embodiment 63. The method of any one of embodiments 1-62, the method including, prior to the administering, detecting an elevated level of CD73 in the subject relative to a standard control.

Embodiment 64. The method of any one of embodiments 1-63, the method including administering an effective amount of a second therapeutic agent.

Embodiment 65. The method of embodiment 64, wherein the second therapeutic agent is a chemotherapeutic agent.

Embodiment 66. The method of embodiment 64 or 65, wherein the second therapeutic agent is a compound.

Embodiment 67. The method of embodiment 66, wherein the compound is a purine receptor antagonist.

Embodiment 68. The method of embodiment 66 or 67, wherein the compound is an A_{2A} adenosine receptor antagonist or A_{2B} adenosine receptor antagonist.

Embodiment 69. The method of embodiment 68, wherein the A_{2A} adenosine receptor antagonist is a thienopyrimidine compound.

Embodiment 70. The method of embodiment 68 or 69, wherein the A_{2A} adenosine receptor antagonist as the structure of formula:

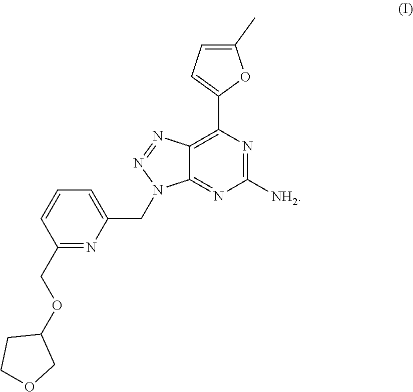 [SMILES: Cc1ccc(-c2nc(N)nc3c2nnn3Cc2cccc(COC3CCOC3)n2)o1]

Embodiment 71. The method of one of embodiments 64-70, wherein the antibody and the second therapeutic agent are administered in a combined synergistic amount.

### REFERENCES

1. Loi et al., Proc Natl Acad Sci USA 2013; 110: 11091-11096.
2. Geoghegan et al., mAbs 2016; 8: 454-467.

INFORMAL SEQUENCE LISTING
(CDR L1) SEQ ID NO: 1:
RASKNVSTSGYSYMH
(CDR L2) SEQ ID NO: 2:
LASNLES
(CDR L3) SEQ ID NO: 3:
QHSRELPFT
(CDR H1) SEQ ID NO: 4:
GYTFTSYWIT
(CDR H2) SEQ ID NO: 5:
PGSGNTNYNEKFKT
(CDR H3) SEQ ID NO: 6:
EGGLTTEDYALDY
Humanized heavy chain variable region SEQ ID NO: 7:
QVQLVQSGAEVEKPGASVKVSCKASGYTFTSYWITWVRQAPGQGLEWMGDIYPGSGN
TNYNEKFKTRVTITADKSTSTAYMELSSLRSEDTAVYYCAKEGGLTTEDYALDYWGQG
TLVTV
BAP094-hum01-LC SEQ ID NO: 8:
GCCATCCAGTTGACCCAGTCTCCATCCTCCCTGTCTGCATCTGTAGGAGACAGAGTC
ACCATCACTTGCAGGGCCAGCAAAAATGTCAGTACATCTGGCTATAGTTATATGCAC
TGGTATCAGCAGAAACCAGGGAAAGCTCCTAAGCTCCTGATCTATCTTGCATCCAAC
CTAGAATCTGGGATCCCACCTCGGTTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum02-LC SEQ ID NO: 9:
GAAATTGTGCTGACTCAGTCTCCAGACTTTCAGTCTGTGACTCCAAAGGAGAAAGTC
ACCATCACCTGCAGGGCCAGCAAAAATGTCAGTACATCTGGCTATAGTTATATGCAC
TGGTATCAGCAGAAACCAGGGAAAGCTCCTAAGCTCCTGATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum03-LC, CPX-003 SEQ ID NO: 10:
GAAATTGTGCTGACTCAGTCTCCAGACTTTCAGTCTGTGACTCCAAAGGAGAAAGTC
ACCATCACCTGCAGGGCCAGCAAAAATGTCAGTACATCTGGCTATAGTTATATGCAC
TGGTATCAGCAGAAACCAGGGAAAGCTCCTAAGCTCCTGATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum04-LC SEQ ID NO: 11:
GAAATTGTGCTGACTCAGTCTCCAGACTTTCAGTCTGTGACTCCAAAGGAGAAAGTC
ACCATCACCTGCAGGGCCAGCAAAAATGTCAGTACATCTGGCTATAGTTATATGCAC
TGGTATCAGCAGAAACCAGGGAAAGCTCCTAAGCTCCTGATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum05-LC, CPX-004 SEQ ID NO: 12:
GATGTTGTGATGACTCAGTCTCCACTCTCCCTGCCCGTCACCCTTGGACAGCCGGCC
TCCATCTCCTGCAGGGCCAGCAAAAATGTCAGTACATCTGGCTATAGTTATATGCAC
TGGTACCAGCAGAAACCTGGCCAGGCTCCCAGGCTCCTCATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum06-LC, CPX-005 SEQ ID NO: 13:
GCCATCCAGTTGACCCAGTCTCCATCCTCCCTGTCTGCATCTGTAGGAGACAGAGTC
ACCATCACTTGCAGGGCCAGCAAAAATGTCAGTACATCTGGCTATAGTTATATGCAC
TGGTACCAGCAGAAACCTGGCCAGGCTCCCAGGCTCCTCATCTATCTTGCATCCAAC
CTAGAATCTGGGGTCCCCTCGAGGTTCAGTGGCAGTGGATCTGGGACAGATTTCACC
TTTACCATCAGTAGCCTGGAAGCTGAAGATGCTGCAACATATTACTGTCAGCACAGT
AGGGAGCTTCCATTCACGTTCGGCCAAGGGACCAAGGTGGAAATCAAA
BAP094-hum07-LC, CPX-006, CPX-007 SEQ ID NO: 14:
GAAATTGTGTTGACACAGTCTCCAGCCACCCTGTCTTTGTCTCCAGGGGAAAGAGCC
ACCCTCTCCTGCAGGGCCAGCAAAAATGTCAGTACATCTGGCTATAGTTATATGCAC
TGGTACCAGCAGAAACCTGGCCAGGCTCCCAGGCTCCTCATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum08-LC SEQ ID NO: 15:
GAAATTGTGTTGACACAGTCTCCAGCCACCCTGTCTTTGTCTCCAGGGGAAAGAGCC
ACCCTCTCCTGCAGGGCCAGCAAAAATGTCAGTACATCTGGCTATAGTTATATGCAC
TGGTACCAGCAGAAACCTGGCCAGGCTCCCAGGCTCCTCATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum09-LC SEQ ID NO: 16:
GAAATTGTGTTGACACAGTCTCCAGCCACCCTGTCTTTGTCTCCAGGGGAAAGAGCC
ACCCTCTCCTGCAGGGCCAGCAAAAATGTCAGTACATCTGGCTATAGTTATATGCAC
TGGTACCAGCAGAAACCTGGCCAGGCTCCCAGGCTCCTCATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum10-LC SEQ ID NO: 17:
GAAATTGTGTTGACACAGTCTCCAGCCACCCTGTCTTTGTCTCCAGGGGAAAGAGCC
ACCCTCTCCTGCAGGGCCAGCAAAAATGTCAGTACATCTGGCTATAGTTATATGCAC
TGGTACCAGCAGAAACCTGGCCAGGCTCCCAGGCTCCTCATCTATCTTGCATCCAAC
CTAGAATCTGGGATCCCACCTCGATTCAGTGGCAGCGGGTATGGAACAGATTTTACC
CTCACAATTAATAACATAGAATCTGAGGATGCTGCATATTACTTCTGTCAGCACAGT
AGGGAGCTTCCATTCACGTTCGGCCAAGGGACCAAGGTGGAAATCAAA
BAP094-hum01-HC SEQ ID NO: 18:
CAGGTTCAGCTGGTGCAGTCTGGAGCTGAGGTGGAGAAGCCTGGGGCCTCAGTGAA
GGTCTCCTGCAAGGCTTCTGGCTACACCTTCACCAGCTACTGGATAACCTGGGTGCG
ACAGGCCCCTGGACAAGGGCTTGAGTGGATGGGTGATATTTATCCTGGTAGTGGTAA
TACTAACTACAATGAGAAGTTCAAGACCAGAGTCACGATTACCGCGGACAAATCCA
CGAGCACAGCCTACATGGAGCTGAGCAGCCTGAGATCTGAGGACACGGCCGTGTAT
TACTGTGCAAAAGAGGGAGGTCTTACTACGGAGGATTATGCTTTGGACTACTGGGGC
CAGGGAACGCTGGTCACCGTCAGCTCA
BAP094-hum02-HC SEQ ID NO: 19:
CAGGTTCAGCTGGTGCAGTCTGGAGCTGAGGTGGAGAAGCCTGGGGCCTCAGTGAA
GGTCTCCTGCAAGGCTTCTGGCTACACCTTCACCAGCTACTGGATAACCTGGGTGCG
ACAGGCCCCTGGACAAGGGCTTGAGTGGATGGGTGATATTTATCCTGGTAGTGGTAA
TACTAACTACAATGAGAAGTTCAAGACCAGAGTCACGATTACCGCGGACAAATCCA
CGAGCACAGCCTACATGGAGCTGAGCAGCCTGAGATCTGAGGACACGGCCGTGTAT
TACTGTGCAAAAGAGGGAGGTCTTACTACGGAGGATTATGCTTTGGACTACTGGGGC
CAGGGAACGCTGGTCACCGTCAGCTCA
BAP094-hum03-HC, CPX-003, CPX-007 SEQ ID NO: 20:
CAGGTTCAGCTGGTGCAGTCTGGAGCTGAGGTGAAGAAGCCTGGGGCCTCAGTGAA
GGTCTCCTGCAAGGCTTCTGGCTACACCTTCACCAGCTACTGGATAACCTGGGTGCG
ACAGGCTCGTGGACAACGCCTTGAGTGGATAGGTGATATTTATCCTGGTAGTGGTAA
TACTAACTACAATGAGAAGTTCAAGACCAGAGTCACGATTACCGCGGACAAATCCA
CGAGCACAGCCTACATGGAGCTGAGCAGCCTGAGATCTGAGGACACGGCCGTGTAT
TACTGTGCAAAAGAGGGAGGTCTTACTACGGAGGATTATGCTTTGGACTACTGGGGC
CAGGGAACGCTGGTCACCGTCAGCTCA
BAP094-hum04-HC SEQ ID NO: 21:
GAGGTCCAGCTGGTACAGTCTGGGGCTGAGGTGAAGAAGCCTGGGGCTACAGTGAA
AATCTCCTGCAAGGTTTCTGGCTACACCTTCACCAGCTACTGGATAACCTGGATCCG
CCAGCCCCCAGGGAAGGGGCTGGAGTGGATTGGTGATATTTATCCTGGTAGTGGTA
ATACTAACTACAATGAGAAGTTCAAGACCAGAGTCACCATCTCAGCCGACAAGTCC
ATCAGCACCGCCTACCTGCAGTGGAGCAGCCTGAAGGCCTCGGACACCGCCATGTA
TTACTGTGCAAAAGAGGGAGGTCTTACTACGGAGGATTATGCTTTGGACTACTGGGG
CCAGGGAACGCTGGTCACCGTCAGCTCA
BAP094-hum05-HC, CPX-004 SEQ ID NO: 22:
GAGGTCCAGCTGGTACAGTCTGGGGCTGAGGTGAAGAAGCCTGGGGCTACAGTGAA
AATCTCCTGCAAGGTTTCTGGCTACACCTTCACCAGCTACTGGATAACCTGGATCCG
CCAGCCCCCAGGGAAGGGGCTGGAGTGGATTGGTGATATTTATCCTGGTAGTGGTA
ATACTAACTACAATGAGAAGTTCAAGACCAGAGTCACCATCTCAGCCGACAAGTCC
ATCAGCACCGCCTACCTGCAGTGGAGCAGCCTGAAGGCCTCGGACACCGCCATGTA
TTACTGTGCAAAAGAGGGAGGTCTTACTACGGAGGATTATGCTTTGGACTACTGGGG
CCAGGGAACGCTGGTCACCGTCAGCTCA
BAP094-hum06-HC, CPX-005, CPX-006 SEQ ID NO: 23:
CAGGTTCAGCTGGTGCAGTCTGGAGCTGAGGTGGAGAAGCCTGGGGCCTCAGTGAA
GGTCTCCTGCAAGGCTTCTGGCTACACCTTCACCAGCTACTGGATAACCTGGGTGCG
ACAGGCCCCTGGACAAGGGCTTGAGTGGATGGGTGATATTTATCCTGGTAGTGGTAA
TACTAACTACAATGAGAAGTTCAAGACCAGAGTCACGATTACCGCGGACAAATCCA
CGAGCACAGCCTACATGGAGCTGAGCAGCCTGAGATCTGAGGACACGGCCGTGTAT
TACTGTGCAAAAGAGGGAGGTCTTACTACGGAGGATTATGCTTTGGACTACTGGGGC
CAGGGAACGCTGGTCACCGTCAGCTCA
BAP094-hum07-HC SEQ ID NO: 24:
CAGGTTCAGCTGGTGCAGTCTGGAGCTGAGGTGGAGAAGCCTGGGGCCTCAGTGAA
GGTCTCCTGCAAGGCTTCTGGCTACACCTTCACCAGCTACTGGATAACCTGGGTGCG
ACAGGCCCCTGGACAAGGGCTTGAGTGGATGGGTGATATTTATCCTGGTAGTGGTAA
TACTAACTACAATGAGAAGTTCAAGACCAGAGTCACGATTACCGCGGACAAATCCA
CGAGCACAGCCTACATGGAGCTGAGCAGCCTGAGATCTGAGGACACGGCCGTGTAT
TACTGTGCAAAAGAGGGAGGTCTTACTACGGAGGATTATGCTTTGGACTACTGGGGC
CAGGGAACGCTGGTCACCGTCAGCTCA
BAP094-hum08-HC SEQ ID NO: 25:
GAGGTCCAGCTGGTACAGTCTGGGGCTGAGGTGAAGAAGCCTGGGGCTACAGTGAA
AATCTCCTGCAAGGTTTCTGGCTACACCTTCACCAGCTACTGGATAACCTGGGTGCG
ACAGGCCCCTGGACAAGGGCTTGAGTGGATGGGTGATATTTATCCTGGTAGTGGTAA
TACTAACTACAATGAGAAGTTCAAGACCAGAGTCACGATTACCGCGGACAAATCCA
CGAGCACAGCCTACATGGAGCTGAGCAGCCTGAGATCTGAGGACACGGCCGTGTAT
TACTGTGCAAAAGAGGGAGGTCTTACTACGGAGGATTATGCTTTGGACTACTGGGGC
CAGGGAACGCTGGTCACCGTCAGCTCA
BAP094-hum09-HC SEQ ID NO: 26:
CAGGTTCAGCTGGTGCAGTCTGGAGCTGAGGTGAAGAAGCCTGGGGCCTCAGTGAA
GGTCTCCTGCAAGGCTTCTGGCTACACCTTCACCAGCTACTGGATAACCTGGGTGCG
ACAGGCTCGTGGACAACGCCTTGAGTGGATAGGTGATATTTATCCTGGTAGTGGTAA
TACTAACTACAATGAGAAGTTCAAGACCAGAGTCACGATTACCGCGGACAAATCCA
CGAGCACAGCCTACATGGAGCTGAGCAGCCTGAGATCTGAGGACACGGCCGTGTAT
TACTGTGCAAAAGAGGGAGGTCTTACTACGGAGGATTATGCTTTGGACTACTGGGGC
CAGGGAACGCTGGTCACCGTCAGCTCA
BAP094-hum10-HC SEQ ID NO: 27:
GAGGTCCAGCTGGTACAGTCTGGGGCTGAGGTGAAGAAGCCTGGGGCTACAGTGAA
AATCTCCTGCAAGGTTTCTGGCTACACCTTCACCAGCTACTGGATAACCTGGATCCG
CCAGCCCCCAGGGAAGGGGCTGGAGTGGATTGGTGATATTTATCCTGGTAGTGGTA
ATACTAACTACAATGAGAAGTTCAAGACCAGAGTCACCATCTCAGCCGACAAGTCC
ATCAGCACCGCCTACCTGCAGTGGAGCAGCCTGAAGGCCTCGGACACCGCCATGTA
TTACTGTGCAAAAGAGGGAGGTCTTACTACGGAGGATTATGCTTTGGACTACTGGGG
CCAGGGAACGCTGGTCACCGTCAGCTCA
BAP094-01-LC CPX-002 (chimeric) SEQ ID NO: 28:
GACATTGTGCTGACACAGTCTCCTGCTTCCTTAGCTGTATCTCTGGGGCAGAGGGCC
ACCATCTCATGCAGGGCCAGCAAAAATGTCAGTACATCTGGCTATAGTTATATGCAC
TGGTACCAACAGAAACCAGGACAGCCACCCAAACTCCTCATCTATCTTGCATCCAAC
CTAGAATCTGGGGTCCCTACCAGGTTCAGTGGCAGTGGGTCTGGGACAGACTTCACC
CTCAACATCCATCCTGTGGAGGAGGAGGATGCTGCAACCTATTACTGTCAGCACAGT
AGGGAGCTTCCATTCACGTTCGGCTCGGGGACAAAGTTGGAAATAAAA
BAP094-01-HC CPX-002 (chimeric) SEQ ID NO: 29:
CAGGTCCAACTGCAGCAGCCTGGGGCTGAGCTTGTGAAGCCTGGGGCTTCAGTGAA
GATGTCCTGCAAGGCTTCTGGCTACACCTTCACCAGCTACTGGATAACCTGGGTGAA
GCAGAGGCCTGGACAAGGCCTTGAGTGGATTGGAGATATTTATCCTGGTAGTGGTA
ATACTAACTACAATGAGAAGTTCAAGACCAAGGCCACACTGACTGTAGACACATCC
TCCAGCACAGCCTACATGCAGCTCAGCAGCCTGACATCTGAGGACTCTGCGGTCTAT
TACTGTGCAAAAGAGGGAGGTCTTACTACGGAGGATTATGCTTTGGACTACTGGGGC
CAGGGAACGCTGGTCACCGTCAGCTCA
BAP094-01-LC SEQ ID NO: 30:
DIVLTQSPASLAVSLGQRATISCRASKNVSTSGYSYMHWYQQKPGQPPKLLIYLASNLES
GVPTRFSGSGSGTDFTLNIHPVEEEDAATYYCQHSRELPFTFGSGTKLEIK
BAP094-hum01-LC SEQ ID NO: 31:
AIQLTQSPSSLSASVGDRVTITCRASKNVSTSGYSYMHWYQQKPGKAPKLLIYLASNLES
GIPPRFSGSGYGTDFTLTINNIESEDAAYYFCQHSRELPFTFGQGTKVEIK
BAP094-hum02-LC SEQ ID NO: 32:
EIVLTQSPDFQSVTPKEKVTITCRASKNVSTSGYSYMHWYQQKPGKAPKLLIYLASNLES
GIPPRFSGSGYGTDFTLTINNIESEDAAYYFCQHSRELPFTFGQGTKVEIK
BAP094-hum03-LC SEQ ID NO: 33:
EIVLTQSPDFQSVTPKEKVTITCRASKNVSTSGYSYMHWYQQKPGKAPKLLIYLASNLES
GIPPRFSGSGYGTDFTLTINNIESEDAAYYFCQHSRELPFTFGQGTKVEI
BAP094-hum04-LC SEQ ID NO: 34:
EIVLTQSPDFQSVTPKEKVTITCRASKNVSTSGYSYMHWYQQKPGKAPKLLIYLASNLES
GIPPRFSGSGYGTDFTLTINNIESEDAAYYFCQHSRELPFTFGQGTKVEIK
BAP094-hum05-LC SEQ ID NO: 35:
DVVMTQSPLSLPVTLGQPASISCRASKNVSTSGYSYMHWYQQKPGQAPRLLIYLASNLES
GIPPRFSGSGYGTDFTLTINNIESEDAAYYFCQHSRELPFTFGQGTKVEIK
BAP094-hum06-LC SEQ ID NO: 36:
AIQLTQSPSSLSASVGDRVTITCRASKNVSTSGYSYMHWYQQKPGQAPRLLIYLASNLES
GVPSRFSGSGSGTDFTFTISSLEAEDAATYYCQHSRELPFTFGQGTKVEIK
BAP094-hum07-LC SEQ ID NO: 37:
EIVLTQSPATLSLSPGERATLSCRASKNVSTSGYSYMHWYQQKPGQAPRLLIYLASNLES
GIPPRFSGSGYGTDFTLTINNIESEDAAYYFCQHSRELPFTFGQGTKVEIK
BAP094-hum08-LC SEQ ID NO: 38:
EIVLTQSPATLSLSPGERATLSCRASKNVSTSGYSYMHWYQQKPGQAPRLLIYLASNLES
GIPPRFSGSGYGTDFTLTINNIESEDAAYYFCQHSRELPFTFGQGTKVEIK
BAP094-hum09-LC SEQ ID NO: 39:
EIVLTQSPATLSLSPGERATLSCRASKNVSTSGYSYMHWYQQKPGQAPRLLIYLASNLES
GIPPRFSGSGYGTDFTLTINNIESEDAAYYFCQHSRELPFTFGQGTKVEIK
BAP094-hum10-LC SEQ ID NO: 40:
EIVLTQSPATLSLSPGERATLSCRASKNVSTSGYSYMHWYQQKPGQAPRLLIYLASNLES
GIPPRFSGSGYGTDFTLTINNIESEDAAYYFCQHSRELPFTFGQGTKVEIK
BAP094-01-HC SEQ ID NO: 41:
QVQLQQPGAELVKPGASVKMSCKASGYTFTSYWITWVKQRPGQGLEWIGDIYPGSGNT
NYNEKFKTKATLTVDTSSSTAYMQLSSLTSEDSAVYYCAKEGGLTTEDYALDYWGQGT
LVTVSS
BAP094-hum01-HC SEQ ID NO: 42:
QVQLVQSGAEVEKPGASVKVSCKASGYTFTSYWITWVRQAPGQGLEWMGDIYPGSGNT
NYNEKFKTRVTITADKSTSTAYMELSSLRSEDTAVYYCAKEGGLTTEDYALDYWGQGT
LVTVSS
BAP094-hum02-HC SEQ ID NO: 43:
QVQLVQSGAEVEKPGASVKVSCKASGYTFTSYWITWVRQAPGQGLEWMGDIYPGSGNT
NYNEKFKTRVTITADKSTSTAYMELSSLRSEDTAVYYCAKEGGLTTEDYALDYWGQGT
LVTVSS
BAP094-hum03-HC SEQ ID NO: 44:
QVQLVQSGAEVKKPGASVKVSCKASGYTFTSYWITWVRQARGQRLEWIGDIYPGSGNT
NYNEKFKTRVTITADKSTSTAYMELSSLRSEDTAVYYCAKEGGLTTEDYALDYWGQGT
LVTVSS
BAP094-hum04-HC SEQ ID NO: 45:
EVQLVQSGAEVKKPGATVKISCKVSGYTFTSYWITWIRQPPGKGLEWIGDIYPGSGNT
NYNEKFKTRVTISADKSISTAYLQWSSLKASDTAMYYCAKEGGLTTEDYALDYWGQGT
LVTVSS
BAP094-hum05-HC SEQ ID NO: 46:
EVQLVQSGAEVKKPGATVKISCKVSGYTFTSYWITWIRQPPGKGLEWIGDIYPGSGNT
NYNEKFKTRVTISADKSISTAYLQWSSLKASDTAMYYCAKEGGLTTEDYALDYWGQGT
LVTVSS
BAP094-hum06-HC SEQ ID NO: 47:
QVQLVQSGAEVEKPGASVKVSCKASGYTFTSYWITWVRQAPGQGLEWMGDIYPGSGNT
NYNEKFKTRVTITADKSTSTAYMELSSLRSEDTAVYYCAKEGGLTTEDYALDYWGQGT
LVTVSS
BAP094-hum07-HC SEQ ID NO: 48:
QVQLVQSGAEVEKPGASVKVSCKASGYTFTSYWITWVRQAPGQGLEWMGDIYPGSGNT
NYNEKFKTRVTITADKSTSTAYMELSSLRSEDTAVYYCAKEGGLTTEDYALDYWGQGT
LVTVSS
BAP094-hum08-HC SEQ ID NO: 49:
EVQLVQSGAEVKKPGATVKISCKVSGYTFTSYWITWVRQAPGQGLEWMGDIYPGSGNT
NYNEKFKTRVTITADKSTSTAYMELSSLRSEDTAVYYCAKEGGLTTEDYALDYWGQGT
LVTVSS
BAP094-hum09-HC SEQ ID NO: 50:
QVQLVQSGAEVKKPGASVKVSCKASGYTFTSYWITWVRQARGQRLEWIGDIYPGSGNT
NYNEKFKTRVTITADKSTSTAYMELSSLRSEDTAVYYCAKEGGLTTEDYALDYWGQGT
LVTVSS
BAP094-hum10-HC SEQ ID NO: 51:
EVQLVQSGAEVKKPGATVKISCKVSGYTFTSYWITWIRQPPGKGLEWIGDIYPGSGNT
NYNEKFKTRVTISADKSISTAYLQWSSLKASDTAMYYCAKEGGLTTEDYALDYWGQGT
LVTVSS
CPX-006_HC (codon-optimized) SEQ ID NO: 52:
AAGCTTGCCGCCACCATGGAATGGTCCTGGGTGTTCCTGTTCTTCCTGTCCGTGACCA
CCGGCGTGCACTCCCAGGTGCAGCTGGTGCAGTCTGGCGCCGAGGTGGAAAAGCCT
GGCGCCTCTGTGAAGGTGTCCTGCAAGGCCTCCGGCTACACCTTTACCAGCTACTGG
ATCACCTGGGTGCGACAGGCTCCTGGACAGGGCCTGGAATGGATGGGCGACATCTA
CCCTGGCTCCGGCAACACCAACTACAACGAGAAGTTCAAGACCCGCGTGACCATCA
CCGCCGACAAGTCCACCTCCACCGCCTACATGGAACTGTCCTCCCTGCGGAGCGAGG
ACACCGCCGTGTACTACTGTGCTAAAGAGGGCGGCCTGACCACCGAGGACTACGCC
CTGGATTATTGGGGCCAGGGCACCCTCGTGACCGTGTCCTCTGCTTCTACCAAGGGC
CCCTCCGTGTTCCCTCTGGCCCCTTCCAGCAAGTCTACCTCTGGCGGCACAGCCGCTC
TGGGCTGCCTCGTGAAGGACTACTTCCCCGAGCCCGTGACAGTGTCTTGGAACTCTG
GCGCCCTGACCAGCGGAGTGCACACCTTCCCTGCTGTGCTGCAGTCCTCCGGCCTGT
ACTCCCTGTCCTCCGTCGTGACTGTGCCCTCCAGCTCTCTGGGCACCCAGACCTACAT
CTGCAACGTGAACCACAAGCCCTCCAACACCAAGGTGGACAAGAAGGTGGAACCCA
AGTCCTGCGACAAGACCCACACCTGTCCCCCTTGTCCTGCCCCTGAACTGCTGGGCG
GACCCTCTGTGTTTCTGTTCCCCCCAAAGCCCAAGGACACCCTGATGATCTCCCGGA
CCCCCGAAGTGACCTGCGTGGTGGTGGATGTGTCCCACGAGGACCCTGAAGTGAAG
TTCAATTGGTACGTGGACGGCGTGGAAGTGCACAACGCCAAGACCAAGCCTAGAGA
GGAACAGTACCAGTCCACCTACCGGGTGGTGTCCGTGCTGACCGTGCTGCACCAGG
ATTGGCTGAACGGCAAAGAGTACAAGTGCAAGGTGTCCAACAAGGCTCTGCCTGCC
CCCATCGAAAAGACCATCTCCAAGGCCAAGGGCCAGCCCCGGGAACCCCAGGTGTA
CACACTGCCCCCTAGCAGGGACGAGCTGACCAAGAACCAGGTGTCCCTGACCTGTC
TCGTGAAAGGCTTCTACCCCTCCGATATCGCCGTGGAATGGGAGTCCAACGGCCAGC
CTGAGAACAACTACAAGACCACCCCCCCTGTGCTGGACTCCGACGGCTCATTCTTTC
TGTACTCCAAGCTGACAGTGGACAAGTCCCGGTGGCAGCAGGGCAACGTGTTCTCCT
GCAGCGTGATGCACGAGGCCCTGCACAACCACTACACCCAGAAGTCCCTGTCCCTG
AGCCCCGGCAAGTGATGAATTC
CPX-006_HC (codon-optimized) SEQ ID NO: 53:
MEWSWVFLFFLSVTTGVHS
QVQLVQSGAEVEKPGASVKVSCKASGYTFTSYWITWVRQAPGQGLEWMGDIYPGSGN
TNYNEKFKTRVTITADKSTSTAYMELSSLRSEDTAVYYCAKEGGLTTEDYALDYWGQG
TLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
CPX-006_LC (codon-optimized) SEQ ID NO: 54:
AAGCTTGCCGCCACCATGTCCGTGCCTACCCAGGTGCTGGGACTGCTGCTGCTGTGG
CTGACCGATGCCAGATGCGAGATCGTGCTGACCCAGTCCCCTGCCACCCTGTCACTG
TCTCCAGGCGAGAGAGCCACCCTGAGCTGCCGGGCCTCCAAGAACGTGTCCACCTC
CGGCTACTCCTACATGCACTGGTATCAGCAGAAGCCCGGCCAGGCCCCCAGACTGCT
GATCTACCTGGCCTCCAACCTGGAATCCGGCATCCCCCCTAGATTCTCCGGCTCTGG
CTACGGCACCGACTTCACCCTGACCATCAACAACATCGAGTCCGAGGACGCCGCCT
ACTACTTCTGCCAGCACTCCAGAGAGCTGCCCTTCACCTTTGGCCAGGGCACCAAGG
TGGAAATCAAGCGGACCGTGGCCGCTCCCTCCGTGTTCATCTTCCCACCTTCCGACG
AGCAGCTGAAGTCCGGCACCGCTTCTGTCGTGTGCCTGCTGAACAACTTCTACCCCC
GCGAGGCCAAGGTGCAGTGGAAGGTGGACAACGCCCTGCAGTCCGGCAACTCCCAG
GAATCCGTGACCGAGCAGGACTCCAAGGACAGCACCTACTCCCTGTCCTCTACCCTG
ACCCTGTCCAAGGCCGACTACGAGAAGCACAAGGTGTACGCCTGCGAAGTGACCCA
CCAGGGCCTGTCTAGCCCCGTGACCAAGTCTTTCAACCGGGGCGAGTGCTGATGAAT
TC
CPX-006_LC (codon-optimized) SEQ ID NO: 55:
MSVPTQVLGLLLLWLTDARC
EIVLTQSPATLSLSPGERATLSCRASKNVSTSGYSYMHWYQQKPGQAPRLLIYLASNLES
GIPPRFSGSGYGTDFTLTINNIESEDAAYYFCQHSRELPFTFGQGTKVEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC

What is claimed is:

1. A method of treating cancer in a subject in need thereof, said method comprising administering to said subject a therapeutically effective amount of a 1E9 antibody, wherein said subject expresses an elevated level of CD73 relative to a standard control, and wherein said 1E9 antibody comprises (i) a mouse CDR L1 as set forth in SEQ ID NO:1, a mouse CDR L2 as set forth in SEQ ID NO:2, a mouse CDR L3 as set forth in SEQ ID NO:3; and (ii) a mouse CDR H1 as set forth in SEQ ID NO:4, a mouse CDR H2 as set forth in SEQ ID NO:5, and a mouse CDR H3 as set forth in SEQ ID NO:6.

2. The method of claim 1, wherein said 1E9 antibody is a humanized 1E9 antibody.

3. The method of claim 2, wherein said humanized 1E9 antibody comprises a humanized light chain variable region and a humanized heavy chain variable region,
wherein said humanized light chain variable region comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87; and
wherein said humanized heavy chain variable region comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

4. The method of claim 1, wherein said elevated level of CD73 has an H-score of at least 150.

5. The method of claim 1, wherein said effective amount of said antibody is administered at a 1:1 ration relative to said elevated level of CD73.

6. The method of claim 1, wherein said antibody is administered at a half maximal effective concentration (EC₅₀) of at least 100 nM.

7. The method of claim 1, wherein said antibody is administered at an EC₅₀ of about 137 nM.

8. The method of claim 1, wherein said antibody is administered at an EC₅₀ of about 189 nM.

9. The method of claim 1, wherein said therapeutically effective amount is about 3 mg/kg, 10 mg/kg, 30 mg/kg, 40 mg/kg, or 120 mg/kg.

10. The method of claim 3, wherein said humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and
wherein said humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

11. The method of claim 3, wherein said humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and
wherein said humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

12. The method of claim 3, wherein said humanized light chain variable region comprises a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and
wherein said humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

13. The method of claim 3 or 12, wherein said humanized light chain variable region comprises a valine at a position corresponding to Kabat position 2, a methionine at a position corresponding to Kabat position 4, a leucine at a position corresponding to Kabat position 9, a proline at a position corresponding to Kabat position 12 and a proline at a position corresponding to Kabat position 18; and
wherein said humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85 and a methionine at a position corresponding to Kabat position 89.

14. The method of claim 3, wherein said humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and
wherein said humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

15. The method of claim 3 or 14, wherein said humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and
wherein said humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

16. The method of claim 3, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

17. The method of claim 3 or 16, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

18. The method of one of claim 3, 16 or 17, wherein said humanized heavy chain variable region comprises the sequence of SEQ ID NO:7.

19. The method of one of claim 3, 16, 17 or 18, wherein said humanized light chain variable region comprises the sequence of SEQ ID NO:37.

20. A method of treating cancer in a subject in need thereof, said method comprising administering to said subject a therapeutically effective amount of a humanized 1E9 antibody, wherein said subject expresses an elevated level of CD73 relative to a standard control and wherein said humanized 1E9 antibody comprises a humanized light chain variable region and a humanized heavy chain variable region, wherein said humanized heavy chain variable region comprises the sequence of SEQ ID NO:7.

21. The method of claim 20, wherein said humanized light chain variable region comprises the sequence of SEQ ID NO:37.

22. The method of claim 1, wherein said antibody is an IgG.

23. The method of claim 1, wherein said antibody is an IgG1.

24. The method o of claim 1, wherein said antibody is an IgG4.

25. The method of claim 1, wherein said antibody is a Fab′ fragment.

26. The method of claim 1, wherein said antibody is a single chain antibody (scFv).

27. The method of claim 1, wherein said antibody is capable of binding a CD73 antigen with an equilibrium dissociation constant (K_{D}) from about 0.3 to about 25 nM.

28. The method of claim 1, wherein said antibody is capable of binding a CD73 antigen with an equilibrium dissociation constant (K_{D}) of about 0.64 nM.

29. The method of claim 1, wherein said antibody is capable of binding a CD73 antigen at a pH of less than about 7.5.

30. The method of claim 1, wherein said antibody is capable of binding a CD73 antigen at a pH from about 6.0 to about 7.0.

31. The method of claim 1, wherein said antibody is capable of binding a CD73 antigen at a pH of about 6.3.

32. The method of claim 1 or 27, wherein said antibody further comprises a glutamine at a position corresponding to Kabat position 297.

33. The method of claim 1, wherein said antibody is bound to a CD73 antigen.

34. The method of claim 33, wherein said CD73 antigen forms part of a cell.

35. The method of claim 34, wherein said cell is a lymphoid cell.

36. The method of claim 34, wherein said cell is a T cell.

37. The method of claim 34, wherein said cell is a cancer cell.

38. A method of treating cancer in a subject in need thereof, said method comprising administering to said subject a therapeutically effective amount of a humanized IgG1 antibody, wherein said subject expresses an elevated level of CD73 relative to a standard control and wherein said humanized IgG1 antibody comprises a humanized light chain variable region and a humanized heavy chain variable region,
wherein said humanized light chain variable region comprises a mouse CDR L1 as set forth in SEQ ID NO:1, a mouse CDR L2 as set forth in SEQ ID NO:2, a mouse CDR L3 as set forth in SEQ ID NO:3; and
wherein said humanized heavy chain variable region comprises a mouse CDR H1 as set forth in SEQ ID NO:4, a mouse CDR H2 as set forth in SEQ ID NO:5, and a mouse CDR H3 as set forth in SEQ ID NO:6.

39. The method of claim 38, wherein said humanized light chain variable region further comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85 or a phenylalanine at a position corresponding to Kabat position 87.

40. The method of claim 38 or 39, wherein said humanized heavy chain variable region further comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 or a glutamic acid at a position corresponding to Kabat position 81.

41. The method of claim 38, wherein said humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and
wherein said humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

42. The method of claim 38, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

43. The method of claim 38, wherein said humanized heavy chain variable region comprises the sequence of SEQ ID NO:7.

44. The method of claim 38, wherein said humanized light chain variable region comprises the sequence of SEQ ID NO:37.

45. The method of claim 38, wherein said antibody further comprises a glutamine at a position corresponding to Kabat position 297.

46. The method of claim 38, wherein said antibody is bound to a CD73 antigen.

47. The method of claim 46, wherein said CD73 antigen forms part of a cell.

48. The method of claim 47, wherein said cell is a T cell.

49. The method of claim 47, wherein said cell is a cancer cell.

50. A method of treating cancer in a subject in need thereof, said method comprising administering to said subject a therapeutically effective amount of an anti-CD73 antibody, wherein said subject expresses an elevated level of CD73 relative to a standard control and wherein said anti-CD73 antibody binds the same epitope as a 1E9 antibody.

51. The method of claim 50, wherein said 1E9 antibody comprises a humanized light chain variable region comprising a mouse CDR L1, mouse CDR L2, or mouse CDR L3 and a humanized heavy chain variable region comprising a mouse CDR H1, mouse CDR H2, or mouse CDR H3.

52. The method of claim 51, wherein said humanized light chain variable region further comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85 or a phenylalanine at a position corresponding to Kabat position 87.

53. The method of claim 51 or 52, wherein said humanized heavy chain variable region further comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80 or a glutamic acid at a position corresponding to Kabat position 81.

54. The method of claim 51, wherein said humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and
wherein said humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87

55. The method of claim 51, wherein said humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

56. The method of claim 51, wherein said humanized heavy chain variable region comprises the sequence of SEQ ID NO:7.

57. The method o of claim 51, wherein said humanized light chain variable region comprises the sequence of SEQ ID NO:37.

58. The method of claim 51, wherein said antibody further comprises a glutamine at a position corresponding to Kabat position 297.

59. The method of claim 51, wherein said antibody is bound to a CD73 antigen.

60. The method of claim 59, wherein said CD73 antigen forms part of a cell.

61. The method of claim 60, wherein said cell is a T cell.

62. The method of claim 60, wherein said cell is a cancer cell.

63. The method of claim 1, said method comprising, prior to said administering, detecting an elevated level of CD73 in said subject relative to a standard control.

64. The method of claim 1, said method comprising administering an effective amount of a second therapeutic agent.

65. The method of claim 64, wherein said second therapeutic agent is a chemotherapeutic agent.

66. The method of claim 64, wherein said second therapeutic agent is a compound.

67. The method of claim 66, wherein said compound is a purine receptor antagonist.

68. The method of claim 66, wherein said compound is an A_{2A} adenosine receptor antagonist or A_{2B} adenosine receptor antagonist.

69. The method of claim 68, wherein said A_{2A} adenosine receptor antagonist is a thienopyrimidine compound.

70. The method of claim 68, wherein said A_{2A} adenosine receptor antagonist as the structure of formula:

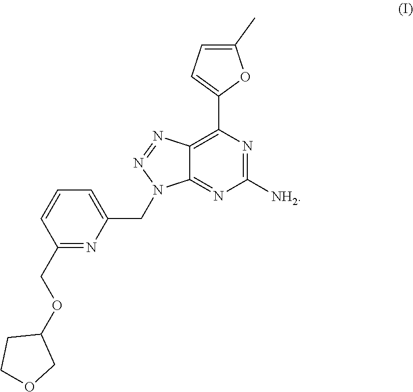 [SMILES: Cc1ccc(-c2nc(N)nc3c2nnn3Cc2cccc(COC3CCOC3)n2)o1]

71. The method of claim 64, wherein said antibody and said second therapeutic agent are administered in a combined synergistic amount.


SEQUENCE LISTING (55 sequences)

SEQ ID NO: 1 - protein, 15 aa, Artificial Sequence
  Synthetic Polypeptide
RASKNVSTSGYSYMH

SEQ ID NO: 2 - protein, 7 aa, Artificial Sequence
  Synthetic Polypeptide
LASNLES

SEQ ID NO: 3 - protein, 9 aa, Artificial Sequence
  Synthetic Polypeptide
QHSRELPFT

SEQ ID NO: 4 - protein, 10 aa, Artificial Sequence
  Synthetic Polypeptide
GYTFTSYWIT

SEQ ID NO: 5 - protein, 14 aa, Artificial Sequence
  Synthetic Polypeptide
PGSGNTNYNEKFKT

SEQ ID NO: 6 - protein, 13 aa, Artificial Sequence
  Synthetic Polypeptide
EGGLTTEDYALDY

SEQ ID NO: 7 - protein, 120 aa, Artificial Sequence
  Synthetic Polypeptide
QVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTV

SEQ ID NO: 8 - DNA, 333 bp, Artificial Sequence
  Synthetic Polynucleotide
gccatccagttgacccagtctccatcctccctgtctgcatctgtaggagacagagtcaccatcacttgcagggccagcaaaaatgtcagtacatctggctatagttatatgcactggtatcagcagaaaccagggaaagctcctaagctcctgatctatcttgcatccaacctagaatctgggatcccacctcggttcagtggcagcgggtatggaacagattttaccctcacaattaataacatagaatctgaggatgctgcatattacttctgtcagcacagtagggagcttccattcacgttcggccaagggaccaaggtggaaatcaaa

SEQ ID NO: 9 - DNA, 333 bp, Artificial Sequence
  Synthetic Polynucleotide
gaaattgtgctgactcagtctccagactttcagtctgtgactccaaaggagaaagtcaccatcacctgcagggccagcaaaaatgtcagtacatctggctatagttatatgcactggtatcagcagaaaccagggaaagctcctaagctcctgatctatcttgcatccaacctagaatctgggatcccacctcgattcagtggcagcgggtatggaacagattttaccctcacaattaataacatagaatctgaggatgctgcatattacttctgtcagcacagtagggagcttccattcacgttcggccaagggaccaaggtggaaatcaaa

SEQ ID NO: 10 - DNA, 333 bp, Artificial Sequence; identical to SEQ ID NO: 9
  Synthetic Polynucleotide

SEQ ID NO: 11 - DNA, 333 bp, Artificial Sequence; identical to SEQ ID NO: 9
  Synthetic Polynucleotide

SEQ ID NO: 12 - DNA, 333 bp, Artificial Sequence
  Synthetic Polynucleotide
gatgttgtgatgactcagtctccactctccctgcccgtcacccttggacagccggcctccatctcctgcagggccagcaaaaatgtcagtacatctggctatagttatatgcactggtaccagcagaaacctggccaggctcccaggctcctcatctatcttgcatccaacctagaatctgggatcccacctcgattcagtggcagcgggtatggaacagattttaccctcacaattaataacatagaatctgaggatgctgcatattacttctgtcagcacagtagggagcttccattcacgttcggccaagggaccaaggtggaaatcaaa

SEQ ID NO: 13 - DNA, 333 bp, Artificial Sequence
  Synthetic Polynucleotide
gccatccagttgacccagtctccatcctccctgtctgcatctgtaggagacagagtcaccatcacttgcagggccagcaaaaatgtcagtacatctggctatagttatatgcactggtaccagcagaaacctggccaggctcccaggctcctcatctatcttgcatccaacctagaatctggggtcccctcgaggttcagtggcagtggatctgggacagatttcacctttaccatcagtagcctggaagctgaagatgctgcaacatattactgtcagcacagtagggagcttccattcacgttcggccaagggaccaaggtggaaatcaaa

SEQ ID NO: 14 - DNA, 333 bp, Artificial Sequence
  Synthetic Polynucleotide
gaaattgtgttgacacagtctccagccaccctgtctttgtctccaggggaaagagccaccctctcctgcagggccagcaaaaatgtcagtacatctggctatagttatatgcactggtaccagcagaaacctggccaggctcccaggctcctcatctatcttgcatccaacctagaatctgggatcccacctcgattcagtggcagcgggtatggaacagattttaccctcacaattaataacatagaatctgaggatgctgcatattacttctgtcagcacagtagggagcttccattcacgttcggccaagggaccaaggtggaaatcaaa

SEQ ID NO: 15 - DNA, 333 bp, Artificial Sequence; identical to SEQ ID NO: 14
  Synthetic Polynucleotide

SEQ ID NO: 16 - DNA, 333 bp, Artificial Sequence; identical to SEQ ID NO: 14
  Synthetic Polynucleotide

SEQ ID NO: 17 - DNA, 333 bp, Artificial Sequence; identical to SEQ ID NO: 14
  Synthetic Polynucleotide

SEQ ID NO: 18 - DNA, 366 bp, Artificial Sequence
  Synthetic Polynucleotide
caggttcagctggtgcagtctggagctgaggtggagaagcctggggcctcagtgaaggtctcctgcaaggcttctggctacaccttcaccagctactggataacctgggtgcgacaggcccctggacaagggcttgagtggatgggtgatatttatcctggtagtggtaatactaactacaatgagaagttcaagaccagagtcacgattaccgcggacaaatccacgagcacagcctacatggagctgagcagcctgagatctgaggacacggccgtgtattactgtgcaaaagagggaggtcttactacggaggattatgctttggactactggggccagggaacgctggtcaccgtcagctca

SEQ ID NO: 19 - DNA, 366 bp, Artificial Sequence; identical to SEQ ID NO: 18
  Synthetic Polynucleotide

SEQ ID NO: 20 - DNA, 366 bp, Artificial Sequence
  Synthetic Polynucleotide
caggttcagctggtgcagtctggagctgaggtgaagaagcctggggcctcagtgaaggtctcctgcaaggcttctggctacaccttcaccagctactggataacctgggtgcgacaggctcgtggacaacgccttgagtggataggtgatatttatcctggtagtggtaatactaactacaatgagaagttcaagaccagagtcacgattaccgcggacaaatccacgagcacagcctacatggagctgagcagcctgagatctgaggacacggccgtgtattactgtgcaaaagagggaggtcttactacggaggattatgctttggactactggggccagggaacgctggtcaccgtcagctca

SEQ ID NO: 21 - DNA, 366 bp, Artificial Sequence
  Synthetic Polynucleotide
gaggtccagctggtacagtctggggctgaggtgaagaagcctggggctacagtgaaaatctcctgcaaggtttctggctacaccttcaccagctactggataacctggatccgccagcccccagggaaggggctggagtggattggtgatatttatcctggtagtggtaatactaactacaatgagaagttcaagaccagagtcaccatctcagccgacaagtccatcagcaccgcctacctgcagtggagcagcctgaaggcctcggacaccgccatgtattactgtgcaaaagagggaggtcttactacggaggattatgctttggactactggggccagggaacgctggtcaccgtcagctca

SEQ ID NO: 22 - DNA, 366 bp, Artificial Sequence; identical to SEQ ID NO: 21
  Synthetic Polynucleotide

SEQ ID NO: 23 - DNA, 366 bp, Artificial Sequence; identical to SEQ ID NO: 18
  Synthetic Polynucleotide

SEQ ID NO: 24 - DNA, 366 bp, Artificial Sequence; identical to SEQ ID NO: 18
  Synthetic Polynucleotide

SEQ ID NO: 25 - DNA, 366 bp, Artificial Sequence
  Synthetic Polynucleotide
gaggtccagctggtacagtctggggctgaggtgaagaagcctggggctacagtgaaaatctcctgcaaggtttctggctacaccttcaccagctactggataacctgggtgcgacaggcccctggacaagggcttgagtggatgggtgatatttatcctggtagtggtaatactaactacaatgagaagttcaagaccagagtcacgattaccgcggacaaatccacgagcacagcctacatggagctgagcagcctgagatctgaggacacggccgtgtattactgtgcaaaagagggaggtcttactacggaggattatgctttggactactggggccagggaacgctggtcaccgtcagctca

SEQ ID NO: 26 - DNA, 366 bp, Artificial Sequence; identical to SEQ ID NO: 20
  Synthetic Polynucleotide

SEQ ID NO: 27 - DNA, 366 bp, Artificial Sequence; identical to SEQ ID NO: 21
  Synthetic Polynucleotide

SEQ ID NO: 28 - DNA, 333 bp, Artificial Sequence
  Synthetic Polynucleotide
gacattgtgctgacacagtctcctgcttccttagctgtatctctggggcagagggccaccatctcatgcagggccagcaaaaatgtcagtacatctggctatagttatatgcactggtaccaacagaaaccaggacagccacccaaactcctcatctatcttgcatccaacctagaatctggggtccctaccaggttcagtggcagtgggtctgggacagacttcaccctcaacatccatcctgtggaggaggaggatgctgcaacctattactgtcagcacagtagggagcttccattcacgttcggctcggggacaaagttggaaataaaa

SEQ ID NO: 29 - DNA, 366 bp, Artificial Sequence
  Synthetic Polynucleotide
caggtccaactgcagcagcctggggctgagcttgtgaagcctggggcttcagtgaagatgtcctgcaaggcttctggctacaccttcaccagctactggataacctgggtgaagcagaggcctggacaaggccttgagtggattggagatatttatcctggtagtggtaatactaactacaatgagaagttcaagaccaaggccacactgactgtagacacatcctccagcacagcctacatgcagctcagcagcctgacatctgaggactctgcggtctattactgtgcaaaagagggaggtcttactacggaggattatgctttggactactggggccagggaacgctggtcaccgtcagctca

SEQ ID NO: 30 - protein, 111 aa, Artificial Sequence
  Synthetic Polypeptide
DIVLTQSPASLAVSLGQRATISCRASKNVSTSGYSYMHWYQQKPGQPPKLLIYLASNLESGVPTRFSGSGSGTDFTLNIHPVEEEDAATYYCQHSRELPFTFGSGTKLEIK

SEQ ID NO: 31 - protein, 111 aa, Artificial Sequence
  Synthetic Polypeptide
AIQLTQSPSSLSASVGDRVTITCRASKNVSTSGYSYMHWYQQKPGKAPKLLIYLASNLESGIPPRFSGSGYGTDFTLTINNIESEDAAYYFCQHSRELPFTFGQGTKVEIK

SEQ ID NO: 32 - protein, 111 aa, Artificial Sequence
  Synthetic Polypeptide
EIVLTQSPDFQSVTPKEKVTITCRASKNVSTSGYSYMHWYQQKPGKAPKLLIYLASNLESGIPPRFSGSGYGTDFTLTINNIESEDAAYYFCQHSRELPFTFGQGTKVEIK

SEQ ID NO: 33 - protein, 110 aa, Artificial Sequence
  Synthetic Polypeptide
EIVLTQSPDFQSVTPKEKVTITCRASKNVSTSGYSYMHWYQQKPGKAPKLLIYLASNLESGIPPRFSGSGYGTDFTLTINNIESEDAAYYFCQHSRELPFTFGQGTKVEI

SEQ ID NO: 34 - protein, 111 aa, Artificial Sequence; identical to SEQ ID NO: 32
  Synthetic Polypeptide

SEQ ID NO: 35 - protein, 111 aa, Artificial Sequence
  Synthetic Polypeptide
DVVMTQSPLSLPVTLGQPASISCRASKNVSTSGYSYMHWYQQKPGQAPRLLIYLASNLESGIPPRFSGSGYGTDFTLTINNIESEDAAYYFCQHSRELPFTFGQGTKVEIK

SEQ ID NO: 36 - protein, 111 aa, Artificial Sequence
  Synthetic Polypeptide
AIQLTQSPSSLSASVGDRVTITCRASKNVSTSGYSYMHWYQQKPGQAPRLLIYLASNLESGVPSRFSGSGSGTDFTFTISSLEAEDAATYYCQHSRELPFTFGQGTKVEIK

SEQ ID NO: 37 - protein, 111 aa, Artificial Sequence
  Synthetic Polypeptide
EIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIK

SEQ ID NO: 38 - protein, 111 aa, Artificial Sequence; identical to SEQ ID NO: 37
  Synthetic Polypeptide

SEQ ID NO: 39 - protein, 111 aa, Artificial Sequence; identical to SEQ ID NO: 37
  Synthetic Polypeptide

SEQ ID NO: 40 - protein, 111 aa, Artificial Sequence; identical to SEQ ID NO: 37
  Synthetic Polypeptide

SEQ ID NO: 41 - protein, 122 aa, Artificial Sequence
  Synthetic Polypeptide
QVQLQQPGAELVKPGASVKMSCKASGYTFTSYWITWVKQRPGQGLEWIGDIYPGSGNTNYNEKFKTKATLTVDTSSSTAYMQLSSLTSEDSAVYYCAKEGGLTTEDYALDYWGQGTLVTVSS

SEQ ID NO: 42 - protein, 122 aa, Artificial Sequence
  Synthetic Polypeptide
QVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSS

SEQ ID NO: 43 - protein, 122 aa, Artificial Sequence; identical to SEQ ID NO: 42
  Synthetic Polypeptide

SEQ ID NO: 44 - protein, 122 aa, Artificial Sequence
  Synthetic Polypeptide
QVQLVQSGAEVKKPGASVKVSCKASGYTFTSYWITWVRQARGQRLEWIGDIYPGSGNTNYNEKFKTRVTITADKSTSTAYMELSSLRSEDTAVYYCAKEGGLTTEDYALDYWGQGTLVTVSS

SEQ ID NO: 45 - protein, 122 aa, Artificial Sequence
  Synthetic Polypeptide
EVQLVQSGAEVKKPGATVKISCKVSGYTFTSYWITWIRQPPGKGLEWIGDIYPGSGNTNYNEKFKTRVTISADKSISTAYLQWSSLKASDTAMYYCAKEGGLTTEDYALDYWGQGTLVTVSS

SEQ ID NO: 46 - protein, 122 aa, Artificial Sequence; identical to SEQ ID NO: 45
  Synthetic Polypeptide

SEQ ID NO: 47 - protein, 122 aa, Artificial Sequence; identical to SEQ ID NO: 42
  Synthetic Polypeptide

SEQ ID NO: 48 - protein, 122 aa, Artificial Sequence; identical to SEQ ID NO: 42
  Synthetic Polypeptide

SEQ ID NO: 49 - protein, 122 aa, Artificial Sequence
  Synthetic Polypeptide
EVQLVQSGAEVKKPGATVKISCKVSGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSS

SEQ ID NO: 50 - protein, 122 aa, Artificial Sequence; identical to SEQ ID NO: 44
  Synthetic Polypeptide

SEQ ID NO: 51 - protein, 122 aa, Artificial Sequence; identical to SEQ ID NO: 45
  Synthetic Polypeptide

SEQ ID NO: 52 - DNA, 1438 bp, Artificial Sequence
  Synthetic Polynucleotide
aagcttgccgccaccatggaatggtcctgggtgttcctgttcttcctgtccgtgaccaccggcgtgcactcccaggtgcagctggtgcagtctggcgccgaggtggaaaagcctggcgcctctgtgaaggtgtcctgcaaggcctccggctacacctttaccagctactggatcacctgggtgcgacaggctcctggacagggcctggaatggatgggcgacatctaccctggctccggcaacaccaactacaacgagaagttcaagacccgcgtgaccatcaccgccgacaagtccacctccaccgcctacatggaactgtcctccctgcggagcgaggacaccgccgtgtactactgtgctaaagagggcggcctgaccaccgaggactacgccctggattattggggccagggcaccctcgtgaccgtgtcctctgcttctaccaagggcccctccgtgttccctctggccccttccagcaagtctacctctggcggcacagccgctctgggctgcctcgtgaaggactacttccccgagcccgtgacagtgtcttggaactctggcgccctgaccagcggagtgcacaccttccctgctgtgctgcagtcctccggcctgtactccctgtcctccgtcgtgactgtgccctccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccctctgtgtttctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtaccagtccacctaccgggtggtgtccgtgctgaccgtgctgcaccaggattggctgaacggcaaagagtacaagtgcaaggtgtccaacaaggctctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattctttctgtactccaagctgacagtggacaagtcccggtggcagcagggcaacgtgttctcctgcagcgtgatgcacgaggccctgcacaaccactacacccagaagtccctgtccctgagccccggcaagtgatgaattc

SEQ ID NO: 53 - protein, 471 aa, Artificial Sequence
  Synthetic Polypeptide
MEWSWVFLFFLSVTTGVHSQVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 739 bp, Artificial Sequence
  Synthetic Polynucleotide
aagcttgccgccaccatgtccgtgcctacccaggtgctgggactgctgctgctgtggctgaccgatgccagatgcgagatcgtgctgacccagtcccctgccaccctgtcactgtctccaggcgagagagccaccctgagctgccgggcctccaagaacgtgtccacctccggctactcctacatgcactggtatcagcagaagcccggccaggcccccagactgctgatctacctggcctccaacctggaatccggcatcccccctagattctccggctctggctacggcaccgacttcaccctgaccatcaacaacatcgagtccgaggacgccgcctactacttctgccagcactccagagagctgcccttcacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctctaccctgaccctgtccaaggccgactacgagaagcacaaggtgtacgcctgcgaagtgacccaccagggcctgtctagccccgtgaccaagtctttcaaccggggcgagtgctgatgaattc

SEQ ID NO: 55 - protein, 238 aa, Artificial Sequence
  Synthetic Polypeptide
MSVPTQVLGLLLLWLTDARCEIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC